(19) United States
(12) Patent Application Publication
(10) Pub. No.: US 2009/0111185 A1
(43) Pub. Date: Apr. 30, 2009

(54) Female genome selection

(75) Inventors: W. Daniel Hillis, Encino, CA (US); Roderick A. Hyde, Redmond, WA (US); Edward K.Y. Jung, Bellevue, WA (US); Robert Langer, Newton, MA (US); Nathan P. Myhrvold, Medina, WA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/978,135

(22) Filed: Oct. 26, 2007

(51) Int. Cl. C12N 15/82; C12N 5/00

(52) U.S. Cl. 435/455; 435/325

(57) ABSTRACT

Systems, methods, compositions and apparatus relating to genome, chromosome, and mitochondria selection are disclosed.

### CROSS-REFERENCE TO RELATED APPLICATIONS

The present application is related to U.S. patent application Ser. No. 11/651,447, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 8 Jan. 2007.

The present application is related to U.S. patent application Ser. No. 11/799,423, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 30 Apr. 2007.

The present application is related to U.S. patent application Ser. No. 11/799,426, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 30 Apr. 2007.

The present application is related to U.S. patent application Ser. No. 11/799,422, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 30 Apr. 2007.

The present application is related to U.S. patent application Ser. No. 11/799,425, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 30 Apr. 2007.

The present application is related to U.S. patent application Ser. No. 11/799,424, entitled SYSTEMS FOR GENOME SELECTION, naming W. Daniel Hillis; Roderick A. Hyde; Edward K. Y. Jung; Robert Langer; Nathan P. Myhrvold and Lowell L. Wood, Jr. as inventors, filed 30 Apr. 2007.

The present application is related to U.S. patent application Ser. No. To Be Assigned, entitled CHROMOSOME SELECTION, naming W. Daniel Hillis; Roderick A. Hyde; Edward K. Y. Jung; Robert Langer; Nathan P. Myhrvold and Lowell L. Wood, Jr. as inventors, filed 25 Oct. 2007 [Attorney Docket No. 0106-004-003A-000000].

The present application is related to U.S. patent application Ser. No. To Be Assigned, entitled MITOCHONDRIAL SELECTION, naming W. Daniel Hillis; Roderick A. Hyde; Edward K. Y. Jung; Robert Langer; Nathan P. Myhrvold and Lowell L. Wood, Jr. as inventors, filed 26 Oct. 2007 [Attorney Docket No. 0106-004-003B-000000].

All subject matter of the Related Applications and of any and all parent, grandparent, great-grandparent, etc. applications of the Related Applications is incorporated herein by reference to the extent such subject matter is not inconsistent herewith.

### SUMMARY

The present application relates, in general, to methods of selecting germ line genomes at least partially based on one or more genetic characteristics of the germ line genomes and related systems implementations, apparatus and/or compositions. Such methods, systems, apparatus, and/or compositions are useful for selecting and/or identifying germ line genomes optionally for use in fertilization. Germ line genomes may be selected to include certain target genetic characteristics and/or to exclude certain target characteristics as optionally determined by a systems operator. Illustrative examples include selection of germ lines that exclude certain genetic characteristics linked with disease risk, and/or that include certain genetic characteristics linked with characteristics selected by the systems operator.

In some aspects, methods for selecting germ line genomes include methods for selecting one or more homologues of one or more chromosomes at least partially based on one or more genetic characteristics of the homologues. In some aspects, methods for selecting germ line genomes include methods for selecting one or more variants of one or more mitochondrial chromosomes at least partially based on one or more genetic characteristics of the variants. Such methods, systems, apparatus, and/or compositions are useful for selecting and/or identifying germ line genomes optionally for use in fertilization, and/or optionally for use in the treatment and/or prevention of one or more diseases or disorders.

Various methods for selecting one or more germ line genomes are disclosed, including but not limited to, various methods for selecting male germ line genomes, female germ line genomes, female-nucleated male germ line cells, nuclear chromosomes, and/or mitochondrial chromosomes. Methods for selecting male germ line genomes (and/or female-nucleated male germ line cells) include, but are not limited to, hybridization-based selection methods, female genetic characteristics-based selection methods, chromatin decondensation-based selection methods, and/or spermatid subtractive determination-based selection methods. Methods for selecting female germ line genomes include, but are not limited to, male genetic characteristics-based selection methods and/or polar body subtractive determination-based selection methods.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1, FIG. 2, and FIG.3 show operational flows representing illustrative embodiments of operations related to determining parameters for selecting one or more reproductive components based on a first possible dataset.

FIG. 4 shows optional embodiments of the operational flow of FIG. 1, FIG. 2, and/or FIG. 3.

FIG. 5 shows optional embodiments of the operational flow of FIG. 1, FIG. 2, and/or FIG. 3.

FIG. 6 shows optional embodiments of the operational flow of FIG. 1, FIG. 2, and/or FIG. 3.

FIG. 7 shows optional embodiments of the operational flow of FIG. 1, FIG. 2, and/or FIG. 3.

FIG. 8 shows optional embodiments of the operational flow of FIG. 1, FIG. 2, and/or FIG. 3.

FIG. 9 shows optional embodiments of the operational flow of FIG. 1, FIG. 2, and/or FIG. 3.

FIG. 10 shows optional embodiments of the operational flow of FIG. 1, FIG. 2, and/or FIG. 3.

FIG. 11, FIG. 12, and FIG. 13 show partial views of an illustrative embodiment of a computer program product that includes a computer program for executing a computer process on a computing device.

FIG. 14 shows an illustrative embodiment of a system in which embodiments may be implemented.

FIG. 15 shows a schematic of an illustrative apparatus in which embodiments may be implemented.

FIG. 16 shows schematics of illustrative embodiments of the apparatus of FIG. 15, with illustrative examples of a sourcing unit.

FIG. 17 shows schematics of illustrative embodiments of the apparatus of FIG. 15, with specific examples of a hybridization unit.

FIG. 18 shows schematics of illustrative embodiments of the apparatus of FIG. 15, with illustrative examples of a monitoring unit.

FIG. 19 shows schematics of illustrative embodiments of the apparatus of FIG. 15, with illustrative examples of a controller unit.

FIG. 20 shows schematics of illustrative embodiments of the apparatus of FIG. 15, with illustrative examples of a computing unit.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present application relates, in general, to systems, apparatus, compositions, and methods of selecting germ line genomes. Those having skill in the art will appreciate that the specific systems, apparatus, compositions, and methods described herein are intended as merely illustrative of their more general counterparts.

As used herein, the term “germ line” means germ cells having genetic material that may be passed to offspring. Germ cells include, but are not limited to, gametogonia (e.g. spermatogonia and oogonia), gametocytes (e.g. spermatocytes and oocytes) and gametes (e.g. spermatozoa and ova).

As used herein, the term “haploid germ line” means germ cells having one set of the genetic material that may be passed to offspring. Haploid germ cells include, but are not limited to, second polar bodies, ova, secondary spermatocytes, spermatids, and spermatozoa. Secondary oocytes are haploid, but have two chromatids for each chromosome.

As used herein the term “homologous chromosomes” and/or “chromosomal homologues” means chromosomes that pair during meiosis. Homologous chromosomes are optionally non-identical chromosomes that contain information for the same biological features and genes at the same loci that optionally provide different genetic information (e.g. alleles). Nuclear chromosomes may be gonosomal (sex chromosomes) and/or autosomal (non-sex chromosomes).

As used herein, the term “genome(s)” means the hereditary information of an organism typically encoded in nucleic acids, either DNA, or RNA, and including both genes and non-coding sequences. The genome may refer to the nucleic acids making up one set of chromosomes of an organism (haploid genome) or both sets of chromosomes of an organism (diploid genome) depending on the context in which it is used. The genome may also include, or be limited to, a mitochondrial genome or a chloroplast genome, for example, depending on the context. The genome may be at least partially isolated, part of a nucleus, and/or in a cell, such as but not limited to, a germ cell or a somatic cell. In some embodiments, one or more genomes may include, but not be limited to, nuclear, organellar, chloroplast and/or mitochondrial genomes.

As used herein, the term “mitochondrial chromosome variants” indicates a different sequence of mitochondrial DNA. Within a subject there is optionally more than one mitochondrial DNA sequence. Each different sequence would be a “variant.” Similarly, within each species, subspecies, or subgrouping there are many mitochondrial DNA variants.

As used herein, the term “genetic characteristic(s)” means any measurable, detectable, and/or identifiable element encoded by, associated with, correlated with, and/or linked to one or more nucleic acid sequences, chromosomal structures, or genomic determinants. The characteristic or element may include, but not be limited to, one or more of a repeat sequence, an inversion, an insertion, a deletion, a substitution, a duplication, a cross-over, a recombination, a SNP, a haplotype, a centromere sequence, a methylation pattern, an epigenetic element, an intron, an exon, a regulatory sequence, an intergenic sequence, and/or a coding or non-coding sequence of nucleotides. The characteristic or element may also include, but not be limited to, allelic markers, alleles, disease markers, genetic abnormalities, genetic diseases, chromosomal abnormalities, genetic mutations and/or protein coding sequences. The characteristic or element may also include, but not be limited to, aspects of mitochondrial nucleic acid sequences and mitochondria. The characteristic or element may also include, but not be limited to, aspects of telomeres including, but not limited to, telomere sequence, telomere repeats and telomere lengths. The characteristic or element may include, but not be limited to, one or more of one or more physical attributes, mental attributes, intellectual attributes, or psychological attributes, or a combination thereof.

As used herein, the term “physical attributes” means any measurable, detectable, and/or identifiable characteristic that may be seen, touched, heard, smelled, or felt or that is involved in one of these processes and is encoded by, associated with, correlated with, and/or linked to one or more nucleic acid sequences, chromosomal structures, or genomic determinants. Examples include, but are not limited to, characteristics associated with height, disease state, body type, hip dysplasia, vision, strength, flexibility, speed, coordination, gait, foot color, lactation, fertility, weight, pelt, skin, skeleto-muscular, longevity, hair, eyes, fur, fleece, wool, hair pattern, hair color, eye color, eye sight, bone length, bone density, skin color, fur thickness, fur color, fur texture (e.g. rough, smooth, thin, thick), fleece color, fleece thickness, wool thickness, and wool color.

As used herein the term “mental attributes” means any measurable, detectable, and/or identifiable characteristics related to the functioning of the mind encoded by, associated with, correlated with, and/or linked to one or more nucleic acid sequences, chromosomal structures, or genomic determinants. Mental attributes may include, but are not limited to intellectual attributes and psychological attributes. Examples include, but are not limited to, intelligence, disposition, mental disorders, depression, insanity, persistence and self-confidence.

The genetic basis for physiology, biochemistry, disease, physical traits, mental traits, intellectual traits, and/or psychological traits of biological entities is known in the art. The genetic basis is determined optionally through associations, correlations and/or linkages among one or more genetic characteristics (Ciba Found. Symp. (1987) 130:215-228). Genetic determinants may be dominant, recessive, partial, and/or multi-factorial. In some embodiments, homozygous alleles may be selected and/or heterozygous alleles may be selected. Additional genetic associations are identifiable using the techniques described in the referenced art.

Illustrative examples of genetic associations, correlations, and/or linkages include, but are not limited to, genetic mechanisms of disease (Nat. Clin. Prat. Rheumatol. (2006) 2:671-678; Curr. Pharm. Des. (2006) 12:3753-3759; Semin. Oncol. (2006) 33:544-551; J. Alzheimers Dis. (2006) 9:45-52; Hum. Mol. Genet. (2006) 15:R117-23; Front. Biosci. (2007) 12:1563-1573; Am. J. Pharmacogenomics (2005) 5:71-92; Front. Biosci. (2007) 12:2670-2682; Autoimmunity (2006) 39:433-444; Nat. Clin. Pract. Endocrinol. Metab. (2006) 2:282-290; Immunogenetics (2006) 58:347-354; BMC Genomics (2006) 7:65; Nat. Rev. Genet. (2006) 7:306-318; Gynecol. Endocrinol. (2006) 22:18-24; Joint Bone Spine (2005) 72:520-526; J. Hypertension (2005) 23:2127-2143; Clin. Sci. (London) (2005) 109:355-364; Front. Biosci. (2006) 11:570-580; Periodontol. 2000 (2005) 39:91-117; Philos. Trans. R. Soc. Lond. B. Biol. Sci. (2005) 360:1529-36), molecular determinants of brain size (Biochem. Biophys. Res. Commun. (2006) 345:911-916), genetic influences on cognition (Philos. Trans. R. Soc. Lond. B. Biol. Sci. (2006) 361:2129-2141; Genes Brain Behavior (2006) 5:44-53; Ment. Retard Dev. Disabil. Res. Rev. (2005) 11:279-285), genetic basis for sleep regulation (Semin. Neurol. (2006) 26:467-483), genetic influences on behavior (Am. J. Psychiatry (2006) 163:1683-1694), genetics of speech (J. Neuroscience (2006) 26:10376-10379); genetic associations for personality (Biol. Psychiatry (2006) October 24; Eur. Neuropsychopharmacol. (2006) August 7; Genes Brain Behav. (2006) 5:240-248); and genetic relationship to athletic performance (Respir. Physiol. Neurobiol. (2006) 151:109-123; Hum. Genet. (2005) 116:331-339; Med. Sci. Sports Exerc. (2006) 38:1863-1888; PLoS Genet. (2005) 1:e42). Illustrative examples of genetic basis for susceptibility and/or resistance for disease include but are not limited to genetic determinants or predispositions for Tay-Sachs disease and sickle cell disease (optionally heterozygous alleles are preferred), as well as modified T cell receptors associated with protection from HIV infection.

As used herein, the term “reference genetic characteristic” means a genetic characteristic that is used as a comparator. Optionally, the comparator can be neutral, desirable, or not desirable. A reference genetic characteristic may be selected for or selected against.

As used herein, the term “target genetic characteristic” means a genetic characteristic that is used as a goal. A target genetic characteristic may be determined by comparison with reference genetic characteristics, for example. A target genetic characteristic may be selected for or selected against, unless context dictates otherwise.

As used herein, the term “weighted analysis” means according one or more target traits and/or genetic characteristics greater, equal or lesser weight based on identifiable criteria. Weighting may be objective, subjective, programmable, and/or user defined.

As used herein, the term “single nucleotide polymorphism(s)” or “SNP(s)” means a nucleic acid sequence variation occurring when a single nucleotide—A, T, C, or G—in the genome (or other shared sequence) differs between members of a species (or between paired chromosomes in an individual). Within a population, SNPs can be assigned a minor allele frequency, the ratio of chromosomes in the population carrying the less common variant to those with the more common variant. SNPs with a minor allele frequency of ≧1% occur every 100 to 300 bases along the human genome, on average, where two of every three SNPs substitute cytosine with thymine. SNPs may fall within coding sequences of genes, noncoding regions of genes, or in the intergenic regions between genes. A SNP within a coding region, in which both forms lead to the same protein sequence, is termed synonymous; if different proteins are produced they are non-synonymous. SNPs that are not in protein coding regions may have consequences for gene splicing, transcription factor binding, or the sequence of non-coding RNA, for example, and/or may indicate the haplotype of the organism.

As used herein, the term “haplotype” means the genetic make up of nucleic acid such as, but not limited to, an individual chromosome, a chromatid, a locus, or an entire genome. In the case of diploid organisms, a genome-wide haplotype comprises one member of the pair of alleles for each locus (that is, half of a diploid genome). A haplotype refers to a set of SNPs on a chromatid that are statistically associated. These associations, and the identification of a few alleles of a haplotype block, can identify other polymorphic sites in its region. Methods for determining haplotypes are known in the art and include, but are not limited to, fluorescent in situ hybridization (FISH) referenced herein.

As used herein, the term “chromosomal characteristic(s)” means normal and abnormal features of chromosomes. Chromosomal characteristics include, but are not limited to, ploidy, translocations, insertions, deletions, rearrangements, and/or mutations. Chromosomal aberrations are frequently associated with lethality and genetic disorders. The numbers of known associations have increased dramatically with the advent of the Human Genomes Project, and have lead to extensive web-based information on genetic disorders. Methods for detecting chromosomal characteristics are known in the art and described herein.

As used herein, the term “nucleic acid(s)” means one or more complex, high-molecular-weight biochemical macromolecules composed of nucleotide chains. Nucleic acids include, but are not limited to, one or more forms of deoxyribonucleic acid (DNA) and ribonucleic acid (RNA). Nucleic acid sequence(s) refers the order of the nucleotides along one or more nucleic acid strands. Methods of determining nucleic acid sequences including target nucleic acid sequences are known in the art. In some embodiments, one or more nucleic acid sequences include, but are not limited to, those that encode one or more proteins, are transcribed into one or more RNA (including, but not limited to, rRNA, tRNA and/or siRNA), are regulatory sequences or repeating sequences, and/or have an at least partially undefined/unknown role. In some embodiments, one or more nucleic acid sequences include, but are not limited to, introns, exons, junk DNA, telomeres and centromeres, pseudogenes and/or hot-spots for duplication of DNA regions.

As used herein, the term “chromatin” means a complex of DNA and protein typically found, for example, inside the nuclei of eukaryotic cells. The nucleic acids are generally in the form of double-stranded DNA except for some germ line cells, or undergoing meiosis or mitosis. In somatic cells and some, but not all, germ line cells, the major proteins involved in chromatin are histones. In some germ line cells, including but not limited to, spermatozoa and some spermatids, the major proteins involved in chromatin are protamines.

As used herein, the term “condensed chromatin” means the more tightly packaged DNA/protein complex that occurs to varying extents during various stages of mitosis & meiosis, for example. During spermiogenesis, spermatid chromatin is remodeled into a more tightly packaged structure where histones are partially or mostly displaced, and partially or completely replaced by protamines (small, arginine-rich proteins). As a result, some but not all spermatids, as well as spermatozoa, have partially or completely condensed chromatin.

As used herein, the term “condensed”, “decondensation”, and/or “recondensation” refers to protamine-based condensation of chromatin unless context dictates otherwise.

As used herein, the term “polyamide” means a molecule, optionally a polymer, containing one or more units, each one optionally a monomer, joined by peptide bonds. The units are optionally natural and/or non-natural amino acids. Although not intended to be limiting, polyamides are understood to bind to nucleic acids, such as DNA, such that the double helix is not disrupted, apparently by binding to the minor or major groove of the double helix.

As used herein, the term “protein nucleic acid” means a nucleic acid with a backbone composed of repeating N-(2-aminoethyl)-glycine units linked by peptide bonds. The various purine and pyrimidine bases are linked to the backbone by methylene carbonyl bonds. PNA binds to DNA by displacing one of the strands and forming Watson-Crick base pairs with the other strand. PNA also binds to RNA by Watson-Crick base pairs.

As used herein, the term “related spermatids” means one or more of the four spermatids that arise during meiosis of a spermatogonium through first and second spermatocytes. The four spermatids that are generated from a single spermatogonium are “related” as used herein. The haplotype of one or more of the related spermatids may be partially and/or completely determined by knowing the haplotype of a related spermatogonium (or any related diploid cell) and the haplotypes of one or more of the other related spermatids. The haplotype of one of the related spermatids may be completely determined by knowing the haplotype of a related spermatogonium (or any related diploid cell) and the haplotypes of the other three related spermatids.

As used herein, the term “related polar bodies” means one or more of the first and second polar bodies that arise during meiosis of a primary oocyte. The three polar bodies that arise from single primary oocyte are “related” as used herein. The haplotypes of one or more of the related polar bodies and/or related ovum can be determined by knowing the haplotype of the primary oocyte (or any related diploid cell) and one or more of the polar body ovum haplotypes. The “related ovum” is the ovum arising from the primary oocyte term which the related polar bodies arose.

As used herein, the term “related female germ line genomes” means a female germ line genome that arises during meiosis of a primary oocyte. Related female germ line genomes include secondary oocytes, ova, and polar bodies, including first polar bodies and second polar bodies.

As used herein, the term “related diploid cell” means a diploid germ line or somatic cell from the same biological entity as a related spermatid or a related polar body.

As used herein, the term “related stem cell” means a germ line stem cell or somatic stem cell from the same biological entity as a germ line cell.

As used herein, the term “related somatic cell” means a somatic cell from the same biological entity as a germ line cell.

As used herein, the term “at least partially” means partially or completely. “Completely” means as near totality as reasonably possible scientifically and/or economically. “Partially” means anything less than completely, but more than none. Partially includes, but is not limited to 99%, 98%, 97%, 96%, 95%, 90%, 85%, 80%, 75%, 70%, 65%, 60%, 55%, 40%, 30%, 25%, 15%, 10%, 5%, 4%, 3%, 2%, and/or 1%. Partially includes, but is not limited to, 1-99, 5-99, 10-99, 25-99, 40-99, 60-99, 80-99, 5-90, 5-75, 5-55, 5-30, 5-15, 5-10, 25-95, 25-85, 25-65, 25-45, 60-90, 60-75, 40-65, and/or 15-35 percent.

As used herein, the term “biological entity” means one or more living entities including, but not limited to, plants, animals, microorganisms, prokaryotes, eukaryotes, protozoa, bacteria, mammals, yeast, E. coli, humans, reptile, insect, bird, amphibian, and/or fish. The animals may include, but are not limited to, domesticated, wild, research, zoo, sports, pet, primate, marine, and/or farm animals. Animals include, but are not limited to, bovine, porcine, swine, ovine, murine, canine, avian, feline, equine, and/or rodent animals. Domesticated and/or farm animals include, but are not limited to, chickens, horses, cattle, pigs, sheep, donkeys, mules, rabbits, goats, ducks, geese, chickens, and/or turkeys. Wild animals include, but are not limited to, non-human primates, bear, deer, elk, raccoons, squirrels, wolves, coyotes, opossums, foxes, skunks, and/or cougars. Research animals include, but are not limited to, rats, mice, hamsters, guinea pigs, rabbits, pigs, dogs, cats and/or non-human primates. Pets include, but are not limited to, dogs, cats, gerbils, hamsters, guinea pigs and/or rabbits. Reptiles include, but are not limited to, snakes, lizards, alligators, crocodiles, iguanas, and/or turtles. Avian animals include, but are not limited to, chickens, ducks, geese, owls, sea gulls, eagles, hawks, and/or falcons. Fish include, but are not limited to, farm-raised, wild, pelagic, coastal, sport, commercial, fresh water, salt water, and/or tropical. Marine animals include, but are not limited to, whales, sharks, seals, sea lions, walruses, penguins, dolphins, and/or fish. One or more of the genomes described herein may be part of or included in one or more biological entities.

As used herein, the term “identifying” means one or more process used to determine one or more components, wherein the one or more components optionally include, but are not limited to, one or more genomes, one or more germ line genomes, one or more chromosomal characteristics, one or more chromosomal homologues, one or more mitochondrial chromosome variants, one or more genetic characteristics, one or more single nucleotide polymorphisms, one or more haplotypes, one or more nucleic acid sequences, one or more genomes, one or more germ line cells, one or more nuclei, etc. and/or other “items” that are appropriate when read in the context in which they occur in the description. Processes include, but are not limited to, user selected, user identified, user determined, software method analysis, algorithm-based, computer mediated, operations research, optimization, simulation, queuing theory, and/or game theory. Illustrative embodiments of such processes include but are not limited to information processing, information technology, datamining, and/or database analysis.

As used herein, the term “separating” means one or more process used to partially or completely isolate from one another one or more components, and/or one or more process that result in one or more components being no longer located in the same place. The one or more components optionally include, but are not limited to, one or more genomes, one or more chromosomal homologues, one or more mitochondrial chromosome variants, one or more germ line cells, one or more nuclei, etc. and/or other components that are appropriate when read in the context in which they occur in the description. Processes include, but are not limited to, manual, automatic, semi-automatic, remote-controlled, and/or robotic. Illustrative embodiments of such processes include but are not limited to fluorescence activated cell sorting (FACS).

As used herein, the term “selecting” means one or more process used to “identify” and/or “separate” one or more components, optionally one or more reproductive components, optionally one or more germ line genomes, optionally one or more genetic characteristics. The one or more components optionally include, but are not limited to, one or more chromosomal characteristics, one or more chromosomal homologues, one or more mitochondrial chromosome variants, one or more genetic characteristics, one or more single nucleotide polymorphisms, one or more haplotypes, one or more nucleic acid sequences, one or more genomes, one or more germ line cells, one or more nuclei, etc. and/or other “items” that are appropriate when read in the context in which they occur in the description. Processes include, but are not limited to, those described above for “identifying” and/or “separating”.

As used herein, the term “selecting for . . . based on” and “selecting against based on” means one or more process used to “identify” and/or “separate” one or more components, optionally one or more reproductive components, and/or optionally one or more germ line genomes, using (or based on) defined parameters. Using (based on) defined parameters may include detecting the presence and/or absence of one or more genetic characteristics, and/or the presence or absence of a weighted combination of one or more genetic characteristics, for example. Using (based on) defined parameters may include detecting the increase and/or decrease of one or more genetic characteristics, and/or the increase or decrease of a weighted combination of one or more genetic characteristics, for example.

As used herein, “presence and/or absence” means detectable and/or not detectable based on scientific and/or economic reasonableness. Something may be detectable and/or undetectable scientifically if a signal is above background and/or below background using a scientifically appropriate assay, and/or if a signal is altered, for example increased and/or decreased, in a statistically significant manner.

As used herein, the term “increase and/or decrease” means a change or alteration (up or down as scientifically appropriate) in the level of detectability as compared with a control and/or reference level, optionally a statistically significant change in the level of detectability as compared with a control and/or reference level.

As used herein, the term “providing and/or co-localizing” means any process resulting in one or more components being in the same place at the same time. By “in the same place at the same time” is meant physical proximity such that the one or more components are capable of interaction on a molecular level. Providing may include, co-localizing, commingling, combining, mixing, assembling, aggregating, injecting, or other similar processes. Methods for providing molecules to the nucleus of living cells are known in the art and include, but are not limited to, microinjection, scrape-loading, bead-loading, osmotic lysis of pinosomes, liposome transfection, and cell permeablization (Journal of Cell Science (1987) 88:669-678; Methods (2003) 29:51-57).

As used herein, the term “removing” and/or “eliminating” includes processes resulting in one or more components being taken out of a particular location. By “being taken out of a particular location” is meant physical separation from a particular place. Physical separation may include destruction of a component (elimination), isolating (e.g. walling off) a component, and/or extraction from the physical location. These processes may apply to a particular component and/or to a larger level component (e.g. a chromosome and/or a nucleus enclosing a chromosome).

As used herein, the term “obtaining” includes processes by which a physical component may be acquired. Components are optionally acquired through purchase and/or special order. Components may be optionally synthesized. Components may be identified optionally by methods described herein, and optionally isolated, extracted and/or removed from a surrounding milieu. Components may be replicated in vitro, in situ, and/or in vivo. These processes may apply to individual components, and/or to a larger level component (e.g. a chromosome and/or a nucleus enclosing a chromosome).

Generic processes useful for co-localizing, providing and/or separating, and including sequential processes, are known in the art and include, but are not limited to, one or more of manual methods, automated or semi-automated methods, robot-controlled methods, remote-controlled methods, mechanical methods, electrical methods, computer and/or software-controlled methods, and fluid flow. Fluid flow includes, but is not limited to, nanofluidics and microfluidics. Nanofluidics and microfluidics include, but are not limited to, continuous flow microfluidics and digital microfluidics, and have been developed for use in biological systems (Annu. Rev. Fluid Mech. (2004) 36:381-411; Annu. Rev. Biomed. Eng. (2002) 4:261-86; Science (1988) 242:1162-1164, Rev. Mod. Phys. (2005) 77:977-1026).

As used herein, the term “differentiation” or the verb form “differentiating” means any process by which cells become a different cell type. Through differentiation, unspecialized or less specialized cells (e.g. pluripotent and/or totipotent cells) become specialized, for example, as regards morphology and/or function. Differentiation may include, but is not limited to, changes in numerous aspects of cell physiology, morphology (e.g. size, shape, polarity), metabolic activity, responsiveness to signals, and/or expression profiles (e.g. gene profiles, protein profiles, lipid profiles, etc.).

As used herein, the term “stem cells” means cells having the ability to renew themselves through mitotic division and that can differentiate into a diverse range of specialized cell types. Examples of stem cells include, but are not limited to, embryonic stem cells, cord blood stem cells, fetal stem cells, adult stem cells, hematopoietic stem cells, mesenchymal stem cells, and epithelial stem cells.

As used herein, the term “hybridization” means one or more processes for co-localizing complementary, single-stranded nucleic acids, and/or co-localizing complementary non-traditional molecules with single- or double-stranded nucleic acids through strand separation and re-annealing, for example. In illustrative embodiments, complementary PNA and/or nucleic acid molecules, optionally oligonucleotides, may hybridize to single- or double-stranded DNA.

Methods for hybridization are known in the art, and include, but are not limited to, conditions for low and high stringency hybridization (Sambrook and Russell. (2001) Molecular Cloning: A Laboratory Manual 3^{rd} edition. Cold Spring Harbor Laboratory Press; Sambrook, Fritsch, Maniatis. Molecular Cloning: A Laboratory Manual 3^{rd} edition. includes a spiral bound, 3 volume set, associated with a web site as an on-line laboratory manual (www.MolecularCloning.com)). Stringency of the hybridization may be controlled (e.g. by the washing conditions) to require up to 100% complementarity between the probe and the target sequence (high stringency), or to allow some mismatches between the probe and the target sequence (low stringency). Factors to determine the appropriate hybridization and wash conditions based on the target and the probe are known in the art. In illustrative embodiments, following the first wash using 0.2×SSC/0.1% SDS for 10 minutes at 68° C., two additional washes with 0.2×SSC/0.1% SDS for 15 minutes each at 68° C. are performed for high stringency washes, two additional washes at 0.2×SSC/0.1% SDS for 15 minutes each at 42° C. for moderate stringency washes, and two additional washes 0.2×SSC/0.1% SDS for 15 minutes each at room temperature for low stringency washes.

As used herein, the term “genotyping” means one or more processes for determining the genotype of one or more biological entities. Methods of genotyping include, but are not limited to, PCR, DNA sequencing, and hybridization to DNA chips or beads. In illustrative embodiments, not intended to be in any way limiting, short tandem repeats, microsatellite DNA, mitochondrial DNA, and/or single nucleotide polymorphisms may be used for genotyping (Forensic Sci. Int. (2004) 146 suppl:S171-3; Forensic Sci. Int. (2005) 50:519-525; Forensic Sci. Int. (2005) 153:237-246; Forensic Sci. Int. (2005) 153:247-259; Forensic Sci. Int. (2005) 154:111-121; Forensic Sci. Int. (2005) 154:181-194; Forensic Sci. Int. (2005) 154:128-136; Forensic Sci. Int. (2006) 157:23-35; Int. J. Legal Med. (2005) 119:10-15; Methods Mol. Biol. (2005) 297:229-242; Electrophoresis (2005) 26:4411-4420; Leg. Med. (Tokyo) (2005) 7:259-262).

As used herein, the term “detecting” means one or more processes for measuring and/or identifying and/or documenting and/or recording the presence or absence and/or amount and/or type and/or intensity of a characteristic, for example, or as appropriate in the context used herein. Methods for detecting molecular genetic alterations are known in the art. Methods include those appropriate for viable or living cells and/or non-viable or non-living cells.

Sequences that include only one base pair change or single nucleotide polymorphism (SNP) can be detected using one or more methods described herein, and/or methods known in the art. Methods for detecting single nucleic acid transcripts, SNPs, and chromosomal abnormalities are known in the art and include, but are not limited to a variety of FISH and other fluorescent techniques (Science (1998) 280:585-590; BioTechniques (2006) 40:489-495). Methods for detecting large scale genetic alterations such as, but not limited to, allelic imbalance, microsatellite instability, insertions, deletions, translocations, and aberrant methylation are known in the art and include, but are not limited to, digital SNP analysis (Clinical Cancer Research (2002) 8:2580-2585).

Methods for detecting specific nucleic acid sequences in viable and/or non-viable cells and/or nuclei are known in the art and include, but are not limited to, using labeled oligonucleotides, labeled protein nucleic acid (PNA) oligonucleotides, and labeled polyamides (Current Organic Chemistry (2006) 10:491-518; Mol. Hum Reprod. (2004) 10:467-472; Mammalian Genome (2000) 11:384-391; Adv. in Genetics (2006) 56:1-51; The EMBO Journal (2003) 22: 6631-6641; Eur. J. Hum. Genetics (2003) 11:337-341; Mammalian Genome (1999) 10: 13-18; The EMBO Journal (2001) 20:3218-3228; Bioorganic & Medicinal Chem. Lett. (2003) 13:1565-1570; Nuc. Acids Res. (2004) 32:2802-2818; Thesis by T. P. Best (2005) California Institute of Technology; Methods (2003) 29:51-57). Quenched probes, such as molecular beacons and quenched auto-ligation probes, provide highly specific detection of nucleic acids, for example (Trends in Biotech. (2005) 23:225-230). Although in some instances, one or more methods are described for RNA, they can be used analogously for DNA.

Methods for imaging nucleic acid molecules, including single nucleic acid molecules, within living cells and/or living cell nuclei are known in the art, and include, but are not limited to, ultra-sensitive optical techniques for imaging fluorescent probes and/or quantum dots (Biochem. Biophys. Res. Commun. (2006) 344:772-779; Histochem. Cell Biol. (2006) 125:451-456; Trends in Cell Biol. (1998) 8:288292; Biophys. J. (2000) 78:2170-2179; Anal. Chem. (2000) 72:5606-5611; Nature (2004) 5:856-862; Science (2004) 304:1797-1800; Biomedical Optics (2005) 10:051406-1 to 051406-9). Although in some instances one or more methods are described for one type of nucleic acid, they can be used analogously for other types of nucleic acid.

As used herein, the term “decondensing” means one or more processes for decreasing and/or reversing the condensation of one or more nucleic acids with proteins, and including for example, but not limited to, decreasing the condensation of chromatin including one or more chromosomes, one or more portions of chromosomes, one or more genomes, or one or more portions of genomes. As used herein, the term “condensing and/or re-condensing” means one or more processes for increasing condensation and/or reversing the decondensation of one or more nucleic acids with proteins including, but not limited to, protamines and optionally histones, and including for example, but not limited to, increasing the condensation of chromatin including one or more chromosomes, one or more portions of chromosomes, one or more genomes, or one or more portions of genomes. In some embodiments, the terms decondensing/recondensing apply specifically to chromatin of spermatids, spermatocytes, and/or spermatozoa that has been partially or completely condensed and/or decondensed in association with protamines and optionally histones.

Methods for decondensing chromatin of spermatids, spermatocytes, and/or spermatozoa that have been partially or completely condensed in association with protamines are known in the art. Methods may be destructive and/or non-destructive of the cells, genomes, and/or nuclei, and may result in viable or non-viable genomes. Methods for partial and/or complete decondensation include, but are not limited to, exposure to dithiothreitol, glutathione, heparin, and/or heparin sulfate, and similar reagents, and one or more of these treatments render sperm still functional for fertilization (J. Cell Science (2005) 118:1811-1820; Hum. Repro. (2005) 20:2784-2789; Theriogenology (2005) 63:783-794; J. Exp. Zool. (1999) 284:789-797; J. Biol. Chem. (2004) 279:20088-20095). Methods for partial and/or complete decondensation of one or more partially and/or completely condensed genomes include exposure to extracts from stimulated ova, exposure to stimulated ova, and/or exposure to recombinant and/or reconstituted extracts of stimulated ova. By stimulated is meant the changes that occur during fertilization.

Methods for identifying genetic characteristics in condensed, partially condensed, partially decondensed, and/or partially recondensed male germ line haploid genomes are known in the art, and non-random chromosome positioning in sperm has been established (J. Cell Science (2005) 118:1811-1820; Biol. Repro. (1993) 48:1193-1201; J. Cell Science (2005) 118:4541-4550).

As used herein, the term “fertilizing” means co-localizing two genomes in a first location such that the genomes form at least one diploid genome including genetic information from both genomes with the potential to become a viable biological entity and/or with the potential to initiate development and/or is totipotent. In some embodiments, at least one genome is a haploid genome. In some embodiments, both genomes are haploid genomes. In some embodiments, at least one genome is a diploid genome. In some embodiments, one or more of the genomes are germ line genomes. In some embodiments, at least one genome is a male germ line genome. In some embodiments, at least one genome is a female germ line genome. In some embodiments, both genomes are female germ line genomes.

Methods for fertilization are known in the art and include, but are not limited to, intracytoplasmic injection of mature and/or immature, damaged and/or undamaged, sperm cells, nuclei, and/or genomes, including, for example, ICSI (Hum. Repro. (2002) 4:990-998; Hum. Repro. (1998) 13:117-127; Reproduction (2005) 130:907-916; Mol. Repro. & Devel. (2004) 68:96-102; Theriogenology (2005) 63:783-794).

As used herein, the term “in vitro” means performing a given action in cells or parts of cells in a controlled environment outside a living biological entity. In vitro actions may be destructive, non-destructive, at least partially destructive, or at least partially non-destructive.

As used herein, the term “destructive” means damaging to the cell or part of a cell such that it no longer is able to be used in the methods described herein, such as selecting, separating, or sorting genomes, and optionally fertilization. Unless contrary to a given context, the term destructive may refer to damage to a cell or part of a cell that that results in a partial or complete loss of viability.

As used herein, the term “non-destructive” means limiting damage to the cell or part of a cell such that it is able to be used in the methods described herein, such as selecting, separating, or sorting genomes, and optionally fertilization. Unless contrary to a given context, the term non-destructive may refer to damage to a cell or part of a cell that that results in partial or no loss of viability.

In one aspect, the disclosure is drawn to one or more methods for selecting one or more germ line genomes, one or more homologues of one or more chromosomes, and/or one or more variants of mitochondrial chromosomes at least partially based on one or more genetic characteristics of one or more germ line genomes. Although one or more methods may be presented separately herein, it is intended and envisioned that one or more methods and/or embodiments of one or more methods may be combined and/or substituted to encompass the full disclosure. In some embodiments, one or more methods described herein are used to generate one or more compositions described herein, and/or are performed on one or more apparatus described herein. In some embodiments, one or more methods may include one or more operations, and using all or more computing devices and/or systems.

In some embodiments, one or more methods include hybridizing one or more probes in vitro to one or more nucleic acid sequences of one or more male germ line haploid genomes; determining one or more genetic characteristics of the one or more male germ line haploid genomes; and selecting one or more of the one or more male germ line haploid genomes based at least partially on one or more of the one or more genetic characteristics of the one or more male germ line haploid genomes.

In some embodiments, one or more methods include detecting one or more genetic characteristics of one or more male germ line haploid genomes at least partially based on methods other than binding of one or more nucleic acids of the one or more male germ line haploid genomes with a polyamide or Hoechst; and selecting one or more of the one or more male germ line haploid genomes based at least partially on the one or more genetic characteristics of the one or more male germ line haploid genomes. In some embodiments, the one or more probes do not include a polyamide.

In some embodiments, one or more methods include detecting one or more genetic characteristics of one or more male germ line haploid genomes at least partially based on sequence-specific binding to one or more nucleic acids of the one or more male germ line haploid genomes, and selecting one or more of the one or more male germ line haploid genomes based at least partially on the one or more genetic characteristics of the one or more male germ line haploid genomes.

In some embodiments, one or more methods include detecting one or more genetic characteristics of one or more male germ line haploid genomes at least partially based on using one or more probes containing one or more nucleic acid elements, and selecting one or more of the one or more male germ line haploid genomes based at least partially on the one or more genetic characteristics of the one or more male germ line haploid genomes.

In some embodiments, one or more methods include detecting one or more genetic characteristics of one or more male germ line haploid genomes at least partially based on using one or more probes that do not bind to the minor groove of DNA, and selecting one or more of the one or more male germ line haploid genomes based at least partially on the one or more genetic characteristics of the one or more male germ line haploid genomes.

In some embodiments, one or more methods include detecting one or more genetic characteristics of one or more male germ line haploid genomes at least partially based on using one or more probes that bind to the major groove of DNA, and selecting one or more of the one or more male germ line haploid genomes based at least partially on the one or more genetic characteristics of the one or more male germ line haploid genomes.

In some embodiments, one or more methods include hybridizing one or more nucleic acid sequence specific probes in vitro to the one or more nucleic acid sequences of the one or more male germ line haploid genomes. In some embodiments, one or more of the one or more probes are selected from the group consisting of a protein nucleic acid and an oligonucleotide.

In some embodiments, one or more methods include determining one or more genetic characteristics of the one or more male germ line haploid genomes at least partially based on detecting the hybridization of the one or more probes in vitro to the one or more nucleic acid sequences of the one or more male germ line haploid genomes.

In some embodiments, one or more methods further include detecting the hybridization of the one or more probes in vitro to the one or more nucleic acid sequences of the one or more male germ line haploid genomes. In some embodiments, detecting the hybridization of the one or more probes in vitro is at least partially based on the presence of a detectable marker of hybridization, the detectable marker of hybridization is optionally selected from the group consisting of quantum dots, molecular beacons, and fluorescence, including but not limited to, fluorescence resonance energy transfer (FRET), and fluorescence in situ hybridization (FISH).

In some embodiments, one or more methods further include analyzing one or more genetic characteristics of the one or more male germ line haploid genomes. In some embodiments, analyzing one or more genetic characteristics includes, but is not limited to, comparing one or more genetic characteristics of one or more male germ line haploid genomes with one or more reference and/or one or more target genetic characteristics. In some embodiments, analyzing one or more genetic characteristics includes, but is not limited to, performing a weighted analysis of one or more of the one or more male germ line haploid genomes at least partially based on a comparison with one or more reference genetic characteristics and/or one or more target genetic characteristics.

In some embodiments, one or more methods include selecting for or against one or more reference and/or one or more target genetic characteristics, and/or a weighted combination of one or more reference and/or one or more target genetic characteristics.

In some embodiments, analyzing one or more genetic characteristics of one or more male germ line haploid genomes includes analyzing optionally a weighted combination of one or more of one or more single nucleotide polymorphisms, one or more chromosomes, or one or more nucleic acid sequences of the one or more male germ line haploid genomes. In some embodiments, one or more methods include determining and/or selecting one or more reference genetic characteristics and/or the one or more target genetic characteristics at least partially based on one or more genetic characteristics of one or more female germ line genomes.

In some embodiments, one or more methods include removing, separating, and/or eliminating one or more of the one or more probes from the one or more male germ line haploid genomes and/or from one or more of the one or more nucleic acid sequences of the one or more male germ line haploid genomes.

In some embodiments, one or more male germ line haploid genomes are at least partially condensed, are part of one or more spermatozoa, and/or are at least partially isolated from one or more spermatozoa. In some embodiments, one or more male germ line haploid genomes are part of one or more spermatids, and/or are at least partially isolated from one or more spermatids.

In some embodiments, one or more genetic characteristics of one or more male germ line haploid genomes include a weighted combination of one or more of the one or more genetic characteristics. In some embodiments, one or more genetic characteristics of the one or more male germ line haploid genomes include one or more single nucleotide polymorphisms, one or more chromosomal characteristics, one or more methylation patterns, one or more DNA sequences, one or more mitochondrial nucleic acid sequences, one or more telomeric sequences, and/or one or more telomeric lengths, optionally selected from the group consisting of total genomic telomeric length, telomeric length of one or more ends of one or more chromosomes, and weighted combinations of one or more telomeric lengths of one or more chromosomes.

In some embodiments, one or more SNPs may identify one or more haplotypes to be selected for or selected against. In some embodiments, the one or more SNPs may alter one or more of one or more coding regions, one or more gene products, one or more non-coding regions, one or more intergenic regions, one or more centromeric regions, one or more telomeric regions, or one or more RNA In some embodiments, the one or more SNPs may be in linkage disequilibrium with one or more traits, one or more alleles, or one or more markers of chromosomal characteristics.

In some embodiments, one or more chromosomal characteristics may include, but are not limited to, one or more duplications, insertions, deletions, substitutions, replications or breaks. In some embodiments, the one or more duplications are of one or more chromosomes (for example, trisomy 21) and/or of portions of one or more chromosomes. In some embodiments, one or more chromosomal characteristics may include, but are not limited to, haplotype and/or nucleic acid sequence.

In some embodiments, one or more nucleic acid sequences may include, but are not limited to, repetitive sequences, telomeric sequences, centromeric sequences, mutated sequences, alternate sequences, intergenic sequences, protein coding sequences, and/or non-coding sequences. In some embodiments, the nucleic acid sequence may be linked with one or more disease or disorder, and optionally may encode a gene linked with one or more disease or disorder.

In some embodiments, one or more methods include selecting, sorting, and/or separating one or more of the one or more male germ line haploid genomes based at least partially on one or more target genetic characteristics.

In some embodiments, one or more methods include selecting, sorting, and/or separating one or more of the one or more male germ line haploid genomes based at least partially on one or more genetic characteristics of one or more female germ line genomes.

In some embodiments, one or more methods include selecting, sorting, and/or separating one or more male germ line haploid genomes based at least partially on one or more genetic characteristics of the one or more male germ line haploid genomes; and wherein at least one of the one or more genetic characteristics of the one or more male germ line haploid genomes is selected, sorted and/or separated at least partially based on one or more genetic characteristics of one or more female genomes, optionally one or more female germ line genomes, optionally one or more female germ line haploid genomes. In some embodiments, one or more methods include determining one or more genetic characteristics of one or more female genomes, optionally one or more female germ line genomes, optionally one or more female germ line haploid genomes; and selecting, separating, and/or sorting one or more male germ line haploid genomes at least partially based on the one or more genetic characteristics of the one or more female germ line genomes, optionally one or more female germ line haploid genomes.

In illustrative embodiments, determining one or more genetic characteristics of one or more female germ line genomes includes, but is not limited to, receiving an input including data representative of the one or more genetic characteristics of the one or more female germ line genomes, where the input may be sent from an external or an internal source. In some illustrative embodiments, the data representative of the one or more genetic characteristics of the one or more female germ line genomes is generated internally. In illustrative embodiments, determining one or more genetic characteristics of one or more female germ line genomes includes, but is not limited to, co-localizing, binding, and/or hybridizing one or more probes and/or one or more molecular markers with one or more nucleic acids of the one or more female germ line genomes.

In some embodiments, the one or more genetic characteristics of the one or more male germ line haploid genomes and/or the one or more female germ line genomes include one or more single nucleotide polymorphisms, one or more chromosomal characteristics, one or more methylation patterns, and/or one or more nucleic acid sequences; or a weighted combination thereof. In some embodiments, one or more genetic characteristics of one or more male germ line haploid genomes and/or female germ line genomes include one or more mitochondrial nucleic acid sequences, one or more telomeric sequences, and/or one or more telomeric lengths, or a weighted combination thereof. The one or more telomeric lengths are optionally selected from the group consisting of a total genomic telomeric length, a telomeric length of one or more ends of one or more chromosomes, and a weighted combination of one or more telomeric lengths of one or more chromosomes.

In some embodiments, one or more SNPs may identify one or more haplotypes to be selected for or selected against. In some embodiments, the one or more SNPs may alter one or more of one or more coding regions, one or more gene products, one or more non-coding regions, one or more intergenic regions, one or more centromeric regions, one or more telomeric regions, or one or more RNA In some embodiments, the one or more SNPs may be in linkage disequilibrium with one or more traits, one or more alleles, or one or more markers of chromosomal characteristics.

In some embodiments, one or more chromosomal characteristics may include, but are not limited to, one or more duplications, insertions, deletions, substitutions, replications or breaks. In some embodiments, the one or more duplications are of one or more chromosomes (for example, trisomy 21) and/or of portions of one or more chromosomes. In some embodiments, one or more chromosomal characteristics may include, but are not limited to, haplotype and/or nucleic acid sequence.

In some embodiments, one or more nucleic acid sequences may include, but are not limited to, repetitive sequences, telomeric sequences, centromeric sequences, mutated sequences, alternate sequences, intergenic sequences, protein coding sequences, and/or non-coding sequences. In some embodiments, the nucleic acid sequence may be linked with one or more disease or disorder, and optionally may encode a gene linked with one or more disease or disorder.

In some embodiments, the one or more genetic characteristics of one or more male germ line haploid genomes and/or one or more female germ line genomes include a weighted combination of the one or more genetic characteristics, optionally including a weighted combination of one or more of one or more single nucleotide polymorphisms, one or more chromosomal characteristics, one or more methylation patterns and/or one or more nucleic acid sequences.

In some embodiments, one or more methods include using the selected one or more male germ line haploid genomes to fertilize one or more eggs containing one or more female germ line genomes. In some embodiments, one or more methods include providing and/or co-localizing the selected one or more male germ line haploid genomes to and/or with the one or more female germ line genomes. In some embodiments, the one or more female germ line genomes are one or more haploid genomes.

In some embodiments, one or more methods further include determining the one or more genetic characteristics of the one or more male germ line haploid genomes and/or the one or more female germ line genomes. In some embodiments, determining the one or more genetic characteristics of the one or more genomes includes detecting one or more nucleic acid sequences of the one or more genomes optionally using one or more polyamides and/or one or more protein nucleic acids.

In some embodiments, determining the one or more genetic characteristics of the one or more male germ line haploid genomes and/or the one or more female germ line genomes includes co-localizing, optionally binding, optionally hybridizing, optionally in vitro, one or more probes and/or one or more molecular markers to one or more nucleic acid sequences of one or more of the one or more genomes. In some embodiments, the one or more probes are one or more nucleic acid specific probes, optionally selected from the group consisting of oligonucleotide, protein nucleic acid, and polyamide.

In some embodiments, determining one or more of the one or more genetic characteristics of the one or more male germ line haploid genomes and/or one or more female germ line genomes is at least partially based on detecting the association, optionally the binding, optionally the hybridization, of the one or more probes and/or one or more molecular markers with the one or more nucleic acid sequences of the one or more genomes.

In some embodiments, one or more methods includes detecting the association, binding, and/or hybridization of the one or more probes and/or one or more molecular markers to the one or more nucleic acid sequences of the one or more male germ line haploid genomes and/or one or more female germ line genomes, optionally by detecting the association, binding, and/or hybridization of the one or more probes based on the presence of a detectable marker of hybridization, the detectable marker of hybridization selected from the group consisting of quantum dots, molecular beacons, and fluorescence, including FRET and/or FISH.

In some embodiments, one or more methods include separating the selected one or more male germ line haploid genomes. In some embodiments, one or more methods include using the selected one or more male germ line haploid genomes to fertilize at least one of the one or more female germ line genomes. In some embodiments, one or more methods include providing and/or co-localizing the selected one or more male germ line haploid genomes to and/or with at least one of the one or more female germ line genomes.

In some embodiments, one or more methods further include analyzing the one or more genetic characteristics of one or more male germ line haploid genomes and/or one or more female germ line genomes. In some embodiments, analyzing one or more genetic characteristics of one or more genomes comprises comparing one or more genetic characteristics of the one or more genomes with one or more reference genetic characteristics and/or target genetic characteristics. In some embodiments, one or more methods include determining, and/or selecting, one or more of the one or more reference genetic characteristics or the one or more target genetic characteristics at least partially based on one or more genetic characteristics of one or more female germ line genomes and/or male germ line genomes. In some embodiments, the one or more reference genetic characteristics and/or target genetic characteristics, and/or a weighted combination thereof, may be selected for or selected against. In some embodiments, analyzing the one or more genetic characteristics of the one or more genomes comprises analyzing one or more single nucleotide polymorphisms, one or more chromosomes, one or more methylation patterns and/or one or more nucleic acid sequences of the one or more genomes.

In some embodiments, the one or more male germ line haploid genomes are part of one or more spermatids, spermatocytes, or spermatozoa. In some embodiments, the one or more male germ line haploid genomes are isolated from one or more spermatids, spermatocytes, or spermatozoa. In some embodiments, the one or more male germ line haploid genomes are at least partially condensed. In some embodiments, the one or more male germ line haploid genomes are from one or more biological entities.

In some embodiments, the one or more female germ line genomes are part of and/or at least partially isolated from one or more of polar bodies, oogonia, or ova. In some embodiments, the one or more female germ line genomes are from one or more biological entities.

In some embodiments, one or more methods include decondensing one or more male germ line haploid genomes; determining one or more genetic characteristics of the one or more male germ line haploid genomes; and selecting, separating, and/or sorting one or more of the one or more male germ line haploid genomes based at least partially on the one or more genetic characteristics of the one or more male germ line haploid genomes.

In some embodiments, one or more male germ line haploid genomes are part of one or more condensed spermatocytes or one or more spermatozoa, and/or are at least partially isolated from one or more condensed spermatocytes or one or more spermatozoa. In some embodiments, one or more male germ line haploid genomes are from one or more biological entities.

In some embodiments, one or more methods include at least partially decondensing one or more male germ line haploid genomes. In some embodiments, one or more methods include decondensing in vitro one or more of the one or more male germ line haploid genomes, optionally by providing one or more reducing agents. In some embodiments, one or more methods include providing one or more reducing agents to one or more of the one or more male germ line haploid genomes. In some embodiments, one or more methods include providing one or more molecular markers to one or more of the one or more male germ line haploid genomes, optionally to one or more decondensed male germ line haploid genomes.

In some embodiments, determining one or more genetic characteristics of the one or more male germ line haploid genomes includes co-localizing, binding, and/or hybridizing, optionally in vitro, one or more, optionally nucleic acid specific, probes and/or with one or more nucleic acid sequences of one or more male germ line haploid genomes.

In some embodiments, determining one or more genetic characteristics of the one or more male germ line haploid genomes includes detecting one or more nucleic acid sequences of the one or more male germ line haploid genomes. In some embodiments, detecting one or more nucleic acid sequences of the one or more male germ line haploid genomes includes detecting one or more molecular markers and/or probes of the one or more nucleic acid sequences of the one or more male germ line haploid genomes. In some embodiments, the one or more molecular markers and/or probes are associated with, bound, and/or hybridized to the one or more nucleic acid sequences of the one or more male germ line haploid genomes.

In some embodiments, determining one or more genetic characteristics of the one or more male germ line haploid genomes includes receiving data representative of the one or more genetic characteristics and/or one or more nucleic acid sequences of the one or more male germ line haploid genomes. Insane embodiments, receiving data may be from an internal and/or an external source and/or input. In some embodiments, determining one or more genetic characteristics of the one or more male germ line haploid genomes includes analyzing the one or more genetic characteristics of the one or more male germ line haploid genomes.

In some embodiments, one or more methods include co-localizing, binding, and/or hybridizing one or more molecular markers and/or probes with one or more nucleic acid sequences of the one or more male germ line haploid genomes.

In some embodiments, one or more methods include detecting one or more nucleic acid sequences of the one or more male germ line haploid genomes.

In some embodiments, one or more methods include analyzing the one or more genetic characteristics of the one or more male germ line haploid genomes. In some embodiments, analyzing the one or more genetic characteristics of the one or more male germ line haploid genomes includes analyzing one or more single nucleotide polymorphisms, one or more chromosomes, one or more methylation patterns, and/or one or more nucleic acid sequences of the one or more male germ line haploid genomes.

In some embodiments, analyzing the one or more genetic characteristics of the one or more male germ line haploid genomes includes comparing the one or more genetic characteristics of one or more male germ line haploid genomes with, optionally a weighted combination of, one or more reference genetic characteristics and/or one or more target genetic characteristics. In some embodiments, the method includes selecting for one or more male germ line haploid genomes with one or more reference genetic characteristics and/or the one or more target genetic characteristics and/or with a weighted combination of one or more reference genetic characteristics and/or one or more target genetic characteristics. In some embodiments, the method includes selecting against one or more male germ line haploid genomes with one or more reference genetic characteristics and/or the one or more target genetic characteristics and/or with a weighted combination of one or more reference genetic characteristics and/or one or more target genetic characteristics.

In some embodiments, one or more methods further include determining and/or selecting one or more reference genetic characteristics and/or the one or more target genetic characteristics at least partially based on one or more genetic characteristics of one or more female germ line genomes. In some embodiments, selecting one or more male germ line haploid genomes includes selecting one or more male germ line haploid genomes at least partially based on one or more genetic characteristics of one or more female germ line genomes.

In some embodiments, one or more methods include separating and/or sorting the selected one or more male germ line haploid genomes. In some embodiments, one or more methods include providing and/or co-localizing the one or more male haploid genomes with one or more female germ line genomes.

In some embodiments, one or more methods include determining one or more genetic characteristics of one or more related spermatid genomes; and selecting, separating, and/or sorting one or more related spermatid genomes based at least partially on one or more genetic characteristics of one or more related spermatid genomes.

In some embodiments, one or more related spermatid genomes are from one or more biological entities. In some embodiments, one or more related spermatid genomes are at least partially isolated from one or more spermatids, and/or are part of one or more spermatids.

In some embodiments, determining one or more genetic characteristics of one or more related spermatid genomes includes subtractively determining one or more genetic characteristics of one or more related spermatid genomes. In some embodiments, subtractively determining one or more genetic characteristics of one or more related spermatid genomes includes determining one or more genetic characteristics of one, two, or three of the one or more related spermatid genomes; and comparing one or more genetic characteristics of one, two, or three of the one or more related spermatid genomes with one or more genetic characteristics of a related diploid genome.

In illustrative embodiments, one or more methods include determining one or more genetic characteristics of one or more related spermatids by determining one or more genetic characteristics of three of the related spermatids, and through a comparative process, determining the one or more genetic characteristics of the fourth related spermatid. In some illustrative embodiments, the comparative process is a subtractive process, where the one or more genetic characteristics of the three related spermatids are compared with the one or more genetic characteristics of the related diploid genomes. The genetic characteristics of the related diploid genomes may be known, or may be determined by sequencing and/or haplotyping, for example.

In some embodiments, determining one or more genetic characteristics of one or more related spermatid genomes includes determining, optionally destructively, one or more genetic characteristics of one or more related diploid genomes, optionally of three related spermatid genomes, optionally of two related spermatid genomes, and/or optionally of one related spermatid genome.

In some embodiments, determining one or more genetic characteristics of one or more related spermatid genomes includes amplifying, optionally destructively, one or more nucleic acid sequences of the one or more related spermatid genomes and/or one or more related diploid genomes. In some embodiments, amplifying one or more nucleic acid sequences of the one or more related spermatid genomes includes amplifying in vitro or in situ the one or more nucleic acid sequences of the one or more related spermatid genomes.

In some embodiments, determining one or more genetic characteristics of one or more related spermatid genomes includes sequencing, optionally destructively, one or more nucleic acids of one or more related diploid genomes and/or one or more related spermatid genomes. In some embodiments, sequencing one or more nucleic acids of the one or more related spermatid genomes includes sequencing in vitro or in situ the one or more nucleic acids of the one or more related spermatid genomes.

In some embodiments, determining one or more genetic characteristics of one or more related spermatid genomes includes co-localizing, binding, and/or hybridizing, optionally destructively, one or more probes and/or one or more molecular markers, optionally nucleic acid sequence specific probes, to one or more nucleic acid sequences of the one or more related spermatid genomes. In some embodiments, co-localizing, binding, and/or hybridizing one or more probes and/or one or more molecular markers to one or more nucleic acid sequences of the one or more related spermatid genomes includes co-localizing, binding, and/or hybridizing one or more probes and/or one or more molecular markers in vitro or in situ to the one or more nucleic acid sequences of the one or more related spermatid genomes.

In some embodiments, determining the one or more genetic characteristics of the one or more related spermatid genomes includes detecting and/or identifying one or more nucleic acid sequences of the one or more related spermatid genomes. In some embodiments, detecting and/or identifying one or more nucleic acid sequences of the one or more related spermatid genomes includes detecting and/or identifying one or more markers of the one or more nucleic acid sequences of the one or more related spermatid genomes, detecting and/or identifying one or more probes associated, bound, and/or hybridized to the one or more nucleic acid sequences of the one or more related spermatid genomes.

In some embodiments, determining one or more genetic characteristics of one or more related spermatid genomes includes receiving data representative of the one or more genetic characteristics of the one or more related spermatid genomes and/or one or more related diploid genomes. In some embodiments, receiving data representative of the one or more genetic characteristics of the one or more related spermatid genomes and/or one or more related diploid genomes includes receiving data representative of one or more nucleic acid sequences of the one or more related spermatid genomes and/or one or more related diploid genomes. In some embodiments, receiving data may include receiving data from one or more internal and/or external sources and/or inputs.

In some embodiments, determining one or more genetic characteristics of one or more related spermatid genomes includes analyzing the one or more genetic characteristics of the one or more related spermatid genomes and/or one or more related diploid genomes.

In some embodiments, one or more methods include sequencing one or more nucleic acids of the one or more related spermatid genomes and/or one or more related diploid genomes. In some embodiments, one or more methods include co-localizing, binding, and/or hybridizing one or more molecular markers and/or one or more probes with one or more nucleic acid sequences of the one or more related spermatid genomes and/or one or more related diploid genomes. In some embodiments, one or more methods include detecting and/or identifying one or more nucleic acid sequences of the one or more related spermatid genomes and/or one or more related diploid genomes.

In some embodiments, one or more methods include analyzing the one or more genetic characteristics of the one or more related spermatid genomes and/or one or more related diploid genomes. In some embodiments, analyzing the one or more genetic characteristics of the one or more related spermatid genomes includes analyzing one or more single nucleotide polymorphisms, one or more chromosomes, one or more methylation patterns, and/or one or more nucleic acid sequences of the one or more related spermatid genomes and/or one or more related diploid genomes.

In some embodiments, determining one or more genetic characteristics of one or more related spermatid genomes at least partially based on the one or more genetic characteristics of one or more related spermatid genomes includes deducing and/or identifying the one or more genetic characteristics of the one or more related spermatid genomes at least partially based on the one or more genetic characteristics of one or more of the one or more related spermatid genomes and/or one or more related diploid genomes.

In some embodiments, analyzing the one or more genetic characteristics of the one or more related spermatid genomes includes comparing the one or more genetic characteristics of the one or more related spermatid genomes with one or more reference genetic characteristics and/or one or more target genetic characteristics, and/or with a weighted combination of one or more reference genetic characteristics and/or one or more target genetic characteristics. In some embodiments, one or more methods include determining and/or selecting one or more reference genetic characteristics and/or the one or more target genetic characteristics at least partially based on one or more genetic characteristics of one or more female genomes optionally one or more female germ line genomes, and/or one or more male genomes, optionally are or more male germ line genomes.

In some embodiments, comparing the one or more genetic characteristics of the one or more related spermatid genomes with one or more reference genetic characteristics and/or one or more target genetic characteristics includes selecting for and/or against one or more related spermatid genomes at least partially based on the presence of one or more reference genetic characteristics and/or one or more target genetic characteristics, and/or the presence of a weighted combination of one or more reference genetic characteristics and/or one or more target genetic characteristics.

In some embodiments, selecting one or more related spermatid genomes includes selecting, sorting, and/or separating one or more related spermatid genomes at least partially based on one or more genetic characteristics of one or more female germ line genomes. In some embodiments, selecting one or more related spermatid genomes at least partially based on one or more genetic characteristics of one or more female germ line genomes includes selecting for and/or against one or more of the one or more related spermatid genomes at least partially based on one or more genetic characteristics of one or more female germ line genomes.

In some embodiments, one or more methods further include separating and/or sorting the selected one or more related spermatid genomes. In some embodiments, one or more methods further include co-localizing and/or providing one or more of the one or more related spermatid genomes with one or more female germ line genomes.

In some embodiments, one or more methods include determining one or more genetic characteristics of one or more related polar body genomes; and selecting, sorting, and/or separating one or more related female germ line genomes based at least partially on the one or more genetic characteristics of the one or more related polar body genomes.

In some embodiments, one or more related polar body genomes and/or one or more related female germ line genomes are from one or more biological entities. In some embodiments, one or more related polar body genomes are at least partially isolated from one or more polar bodies and/or are part of one or more polar bodies. In some embodiments, one or more related polar body genomes are one or more first polar body genomes and/or one or more second polar body genomes.

In some embodiments, one or more related female germ line genomes are at least partially isolated from one or more cells and/or are part of one or more cells. In some embodiments, one or more of the one or more related female germ line genomes are at least partially isolated from one or more ova, and/or are part of one or more ova. In some embodiments, one or more related female germ line genomes are at least partially isolated from one or more related polar bodies and/or are part of one or more related polar bodies.

In some embodiments, one or more methods further include determining one or more genetic characteristics of one or more related female germ line genomes. In some embodiments, determining one or more genetic characteristics of one or more related female germ line genomes includes, but is not limited to, determining one or more genetic characteristics of one or more related polar body genomes. In some embodiments, determining one or more genetic characteristics of one or more related female germ line genomes includes, but is not limited to, subtractively determining one or more genetic characteristics of one or more related female germ line genomes. In some embodiments, subtractively determining one or more genetic characteristics of one or more related female germ line genomes includes, but is not limited to, determining one or more genetic characteristics of one, two or three related polar body genomes; and comparing the one or more genetic characteristics of one, two or three related polar body genomes with one or more one or more genetic characteristics of a related diploid genome. In some embodiments, the one or more one or more genetic characteristics of a related diploid genome are already determined and/or known, or are determined by sequencing and/or haplotyping, for example.

In illustrative embodiments, one or more methods include determining one or more genetic characteristics of one or more related female germ line genomes by determining one or more genetic characteristics of three of the related polar body genomes, and through a comparative process, determining the one or more genetic characteristics of the fourth related female germ line haploid genome. In illustrative embodiments, one or more methods include determining one or more genetic characteristics of one or more related female germ line genomes by determining one or more genetic characteristics of two of the related polar body genomes, and through a comparative process, at least partially determining one or more of the one or more genetic characteristics of the related female germ line diploid genome. In some illustrative embodiments, the comparative process is a subtractive process, where the one or more genetic characteristics of the two or three related polar body genomes are compared with the one or more genetic characteristics of the related diploid genomes.

In some embodiments, determining, optionally destructively, one or more genetic characteristics of one or more related polar body genomes includes determining one or more genetic characteristics of one or more related diploid genomes, of optionally three related polar body genomes, of optionally two related polar body genomes, and/or of optionally one related polar body genome.

In some embodiments, determining one or more genetic characteristics of one or more related polar body genomes includes amplifying, optionally destructively, one or more nucleic acid sequences of the one or more related polar body genomes and/or one or more related diploid genomes. In some embodiments, amplifying one or more nucleic acid sequences of the one or more related polar body genomes includes amplifying in vitro and/or in situ the one or more nucleic acid sequences of the one or more related polar body genomes.

In some embodiments, determining one or more genetic characteristics of one or more related polar body genomes includes sequencing, optionally destructively, one or more nucleic acids of the one or more related polar body genomes and/or one or more related diploid genomes. In some embodiments, sequencing one or more nucleic acids of the one or more related polar body genomes includes sequencing in vitro and/or in situ the one or more nucleic acids of the one or more related polar body genomes.

In some embodiments, determining one or more genetic characteristics of one or more related polar body genomes includes co-localizing, binding, and/or hybridizing, optionally destructively, one or more probes and/or one or more molecular markers to one or more nucleic acid sequences of the one or more related polar body genomes and/or one or more related diploid genomes. In some embodiments, co-localizing, binding, and/or hybridizing one or more probes and/or one or more molecular markers to one or more nucleic acid sequences of the one or more related polar body genomes includes hybridizing the one or more probes and/or molecular markers, optionally nucleic acid sequence specific probes, in vitro and/or in situ to the one or more nucleic acid sequences of the one or more related polar body genomes.

In some embodiments, determining the one or more genetic characteristics of the one or more related polar body genomes includes detecting, and/or identifying optionally destructively, one or more nucleic acid sequences of the one or more related polar body genomes and/or one or more related diploid genomes. In some embodiments, detecting and/or identifying one or more nucleic acid sequences of the one or more related polar body genomes includes detecting and/or identifying one or more markers of the one or more nucleic acid sequences, and/or one or more probes and/or one or more molecular markers co-localized, bound, and/or hybridized to the one or more nucleic acid sequences of the one or more related polar body genomes.

In some embodiments, determining one or more genetic characteristics of one or more related polar body genomes includes receiving data representative of the one or more genetic characteristics and/or one or more nucleic acid sequences of the one or more related polar body genomes and/or one or more related diploid genomes. In some embodiments, receiving data includes receiving data from one or more internal and/or external sources and/or inputs.

In some embodiments, determining one or more genetic characteristics of one or more related polar body genomes includes analyzing the one or more genetic characteristics of the one or more related polar body genomes and/or one or more related diploid genomes. In some embodiments, determining one or more genetic characteristics of one or more related female germ line genomes at least partially based on the genetic characteristics of one or more of the one or more related polar body genomes includes deducing and/or identifying the one or more genetic characteristics of the one or more related female germ line genomes at least partially based on the genetic characteristics of one or more of the one or more related polar body genomes and/or one or more related diploid genomes.

In some embodiments, one or more methods include sequencing one or more nucleic acids of the one or more related polar body genomes and/or one or more related diploid genomes. In some embodiments, one or more methods include co-localizing, binding, and/or hybridizing one or more molecular markers and/or one or more probes with one or more nucleic acid sequences of the one or more related polar body genomes and/or one or more related diploid genomes. In some embodiments, one or more methods include detecting and/or identifying one or more nucleic acid sequences of the one or more related polar body genomes and/or one or more related diploid genomes.

In some embodiments, one or more methods include analyzing the one or more genetic characteristics of the one or more related polar body genomes and/or one or more related diploid genomes. In some embodiments, analyzing the one or more genetic characteristics of the one or more related polar body genomes includes analyzing one or more single nucleotide polymorphisms, one or more chromosomes, one or more methylation patterns, and/or one or more nucleic acid sequences of the one or more related polar body genomes and/or one or more related diploid genomes.

In some embodiments, analyzing the one or more genetic characteristics of the one or more related polar body genomes includes comparing the one or more genetic characteristics of the one or more related polar body genomes with one or more reference genetic characteristics and/or one or more target genetic characteristics, and/or with a weighted combination of one or more reference genetic characteristics and/or one or more target genetic characteristics. In some embodiments, one or more methods include determining and/or selecting one or more reference genetic characteristics and/or one or more target genetic characteristics at least partially based on one or more genetic characteristics of one or more male genomes optionally one or more male germ line genomes and/or one or more female genomes optionally female germ line genomes.

In some embodiments, comparing the one or more genetic characteristics of the one or more related female germ line genomes with one or more reference genetic characteristics and/or one or more target genetic characteristics includes selecting for and/or against one or more related female germ line genomes at least partially based on the presence of one or more reference genetic characteristics and/or the one or more target genetic characteristics, and/or the presence of a weighted combination of one or more reference genetic characteristics and/or the one or more target genetic characteristics.

In some embodiments, selecting one or more related female germ line genomes includes selecting, sorting, and/or separating one or more related female germ line genomes at least partially based on one or more genetic characteristics of one or more male germ line haploid genomes. In some embodiments, one or more methods include separating the selected one or more related female germ line genomes. In some embodiments, one or more methods include co-localizing one or more of the one or more related female germ line genomes with one or more male germ line haploid genomes. In some embodiments, one or more methods include providing one or more of the one or more related female germ line genomes to one or more male germ line haploid genomes.

In some embodiments, one or more methods include selecting one or more homologues of one or more chromosomes at least partially based on one or more genetic characteristics of the one or more homologues; and optionally providing the selected one or more chromosomal homologues to one or more (e.g. first) germ line cells. Methods, apparatus, and/or systems described for one or more other embodiments of the invention are also applicable to this embodiment of the invention unless in conflict.

In some embodiments, one or more methods optionally include obtaining one or more chromosomes optionally from one or more (e.g. third) germ line cells. In some embodiments, one or more methods optionally include removing one or more chromosomes optionally from one or more (e.g. first) germ line cells. In some embodiments, one or more methods optionally include providing and/or co-localizing the one or more (e.g. first) germ line cells to and/or with one or more (e.g. second) germ line cells optionally for fertilization. In some embodiments, one or more of the one or more germ lines cells, for example first, second, and/or third germ line cells may be the same germ line cells or one or more may be different germ line cells, based on context.

In some embodiments, the one or more chromosomes are one or more of autosomal chromosomes and/or gonosomal chromosomes. In some embodiments, the one or more chromosomes are from one or more biological entities. In some embodiments, the one or more chromosomes include, by are not limited to, chromosome I, chromosome II, chromosome IIII, chromosome IV, chromosome V, chromosome VI, chromosome VII, chromosome VIII, chromosome IX, chromosome X, chromosome XI, chromosome XII, chromosome XIII, chromosome XIV, chromosome XV, chromosome XVI, chromosome XVII, chromosome XVIII, chromosome XIX, chromosome XX, chromosome XXI, chromosome XXII, and/or XXIII, etc., optionally from human cells.

Similarly, in illustrative embodiments, the one or more chromosomes may include, but are not limited to the individual chromosomes of one or more biological entities. One or more chromosomes may include chromosomes I through XVIII of domestic cats and/or pigs, chromosomes I through VIII of guinea pigs, chromosomes I through XX of lab mice, chromosomes I through XXI of lab rats, chromosomes I through XXII of rabbits and/or Syrian hamsters, chromosomes I through XXIII of hares, chromosomes I through XXIV of gorillas and/or chimpanzees, chromosomes I through XXVII of domestic sheep, chromosomes I through XXVIII of elephants, chromosomes I through XXX of cows, chromosomes I through XXXI of donkeys, chromosomes I through XXXII of horses, and/or chromosomes I through XXXIX of dogs.

In some embodiments, one or more methods optionally include sorting and/or separating one or more homologues of one or more chromosomes at least partially based on one or more genetic characteristics of the one or more homologues. In some embodiments, one or more methods include separating and/or sorting the selected one or more chromosomal homologues optionally from one or more non-selected chromosomal homologues, and/or one or more non-selected chromosomes.

In illustrative embodiments, one or more chromosomal homologues are separated/sorted from other chromosomal homologues and/or other chromosomes optionally while they are within one or more cells. For example, one or more cells containing one or more selected chromosomal homologues may be sorted and/or separated from other cells containing non-selected chromosomal homologues and/or chromosomes. In illustrative embodiments, one or more chromosomal homologues are separated/sorted from other chromosomal homologues and/or other chromosomes optionally following removal from one or more cells. Methods for separating/sorting are known in the art and/or described herein.

In some embodiments, one or more methods optionally include providing and/or co-localizing one or more chromosomal homologues to and/or with one or more germ line cells. In illustrative embodiments, one or more chromosomes (and/or chromosomal homologues) optionally within a nucleus are provided to one or more optionally enucleated germ line cells. In illustrative embodiments, one or more chromosomes (and/or chromosomal homologues) may be co-localized in a cell, optionally in a nucleus of a cell. In some embodiments, one or more germ line cells may include, but are not limited to, one or more haploid germ line cells, one or more diploid germ line cells, one or more stem cells, one or more spermatogonia, one or more oogonia, one or more oocytes (primary and/or secondary), one or more ova, one or more spermatids, one or more spermatocytes, and/or one or more spermatozoa (optionally through decondensation and chromosome delivery, optionally followed by recondensation).

In some embodiments, one or more methods optionally include providing and/or co-localizing one or more chromosomal homologues to and/or with one or more stem cells, somatic cells, and/or proliferating cells, optionally pluripotent cells. In some embodiments, the one or more stem cells are optionally one or more germ line stem cells and/or one or more somatic stem cells. In some embodiments, the one or more stem cells, somatic cells, and/or pluripotent cells are differentiated to one or more germ line cells. In some embodiments, the one or more chromosomal homologues are removed from one or more stem cells, somatic cells, and/or pluripotent cells and provided to one or more germ line cells.

In some embodiments, one or more methods include differentiating one or more germ line stem cells (or further differentiating one or more intermediate germ line cells). In some embodiments, the one or more germ line stem cells are differentiated to one or more diploid or haploid germ line cells, such as, but not limited to spermatocytes, spermatids, spermatozoa, oocytes (primary and/or secondary), and/or ova. Methods for differentiating germ line cells in vitro, in situ, and/or in vivo are known in the art and/or described herein.

In some embodiments, one or more methods include differentiating one or more stem cells (optionally including pluripotent and/or totipotent cells) to one or more germ line cells. In some embodiments, the one or more stem cells include, but are not limited to fetal stem cells, embryonic stem cells, placental stem cells, cord blood stem cells, bone marrow stem cells (e.g. mesenchymal stem cells), among others. In some embodiments, the one or more stem cells are optionally somatic stem cells. Methods for differentiating stem cells in vitro, in situ, and/or in vivo are known in the art and/or described herein.

In some embodiments, the one or more selected chromosomal homologues are provided to (and/or co-localized in) one or more somatic cells, optionally one or more stem cells and/or progenitor cells. In some embodiments, the one or more somatic cells (e.g. stem cells) are then differentiated to one or more germ line cells. In some embodiments, the one or more chromosomal homologues are transferred from one or more somatic cell to one or more germ line cells.

In some embodiments, one or more methods include obtaining one or more homologues of one or more chromosomes. In some embodiments, obtaining one or more chromosomal homologues includes identifying, purchasing, isolating, extracting, synthesizing, removing, and/or replicating, one or more chromosomal homologues. The one or more chromosomal homologues are optionally obtained from one or more germ line cells (e.g. haploid germ cells, diploid germ cells, stem cells, spermatogonia, oogonia, oocytes (e.g. primary and/or secondary), spermatids, spermatocytes, and/or polar bodies), one or more somatic cells (e.g. stem cells and/or progenitor cells), and/or other appropriate cells known in the art. Methods for obtaining, maintaining, and/or proliferating one or more chromosomes are known in the art and/or described herein.

In illustrative embodiments, one or more chromosomal homologue may initially be identified in one or more cells during non-destructive cell sorting, for example. Depending on the type of cells, they may be encouraged to proliferate, or the nucleus and/or chromosome may be transferred to a proliferating cells for maintenance and/or replication. Optionally, the chromosomal homologue may be extracted and re-inserted into a cell of choice, optionally a germ line cell. This procedure may be performed for one chromosome, two chromosomes, a few chromosomes, several chromosomes, many chromosomes, and/or for an entire genome.

In some embodiments, obtaining one or more chromosomal homologues includes obtaining one or more homologues of: at least one chromosome, at least two chromosomes, at least three chromosomes, at least four chromosomes, at least five chromosomes, at least six chromosomes, at least seven chromosomes, at least eight chromosomes, at least nine chromosomes, at least ten chromosomes, at least eleven chromosomes, at least twelve chromosomes, at least thirteen chromosomes, at least fourteen chromosomes, at least fifteen chromosomes, at least sixteen chromosomes, at least seventeen chromosomes, at least eighteen chromosomes, at least nineteen chromosomes, at least twenty chromosomes, at least twenty-one chromosomes, at least twenty-two chromosomes, or at least twenty-three chromosomes, etc.

In some embodiments, obtaining one or more chromosomal homologues includes obtaining one or more homologues of: chromosome I, chromosome II, chromosome IIII, chromosome IV, chromosome V, chromosome VI, chromosome VII, chromosome VIII, chromosome IX, chromosome X, chromosome XI, chromosome XII, chromosome XIII, chromosome XIV, chromosome XV, chromosome XVI, chromosome XVII, chromosome XVIII, chromosome XIX, chromosome XX, chromosome XXI, chromosome XXII, and/or XXIII, etc.

In some embodiments, obtaining one or more chromosomal homologues includes obtaining at most two homologues of: chromosome I, chromosome II, chromosome 1111, chromosome IV, chromosome V, chromosome VI, chromosome VII, chromosome VIII, chromosome IX, chromosome X, chromosome XI, chromosome XII, chromosome XIII, chromosome XIV, chromosome XV, chromosome XVI, chromosome XVII, chromosome XVIII, chromosome XIX, chromosome XX, chromosome XXI, chromosome XXII, and/or XXIII, etc.

In some embodiments, the one or more methods include removing, isolating, extracting, and/or eliminating one or more chromosomal homologues from one or more germ cell lines. In some embodiments, the one or more homologues are removed, isolated, extracted, and/or eliminated from one or more stem cells, proliferating cells, and/or somatic cells, among others. In illustrative embodiments, one or more chromosomal homologues are optionally removed from one or more cells in order to facilitate the re-insertion of one or more selected chromosomal homologues. In illustrative embodiments, individual chromosomal homologues are optionally removed and/or the intact (or partially intact nucleus) may be removed. Methods for removing, eliminating, extracting, and/or isolating one or more chromosomal homologues are known in the art and/or described herein.

In some embodiments, one or more methods include selecting one or more homologues of one or more chromosomes at least partially based on one or more genetic characteristics of the one or more homologues. Genetic characteristics and methods for selection based on one or more genetic characteristics are known in the art and described herein. Methods described for other embodiments herein are specifically intended as applicable to this embodiment, to the extent that such methods are not inconsistent. In some embodiments, selecting one or more chromosomal homologues at least partially based on one or more genetic characteristics of the one or more homologues includes identifying and/or analyzing one or more chromosomal homologues at least partially based on one or more genetic characteristics. In some embodiments, selection is at least partially based on one or more alleles of one or more genes of the one or more chromosomal homologues.

In illustrative embodiments, selection of chromosomal homologues is at least partially based on one or more alleles of one or more independent genes located on a chromosome and/or one or more alleles of one or more gene systems located on a chromosome. As used herein, the term “gene system” may include, but is not limited to, genes that are associated and/or linked (optionally transcriptionally, translationally, for activity, for function, among others), genes that are associated with one or more common traits, disorders, and/or diseases, genes whose gene products interact, are associated, and/or affect a common trait, disorder and/or disease. Gene systems may be encompassed on a single chromosome, and/or located on one or more, two or more, and/or multiple chromosomes.

In some embodiments, selecting one or more chromosomal homologues at least partially based on one or more genetic characteristics of the one or more homologues includes selecting additional chromosomal homologues (e.g. one or more second, third, fourth, fifth, etc chromosomal homologues). In some embodiments, additional chromosomal homologues are selected at least partially based on one or more genetic characteristics of the additional chromosomal homologues. In some embodiments, additional chromosomal homologues are selected at least partially based on the genetic characteristics of already-selected (or previously selected) chromosomal homologues and/or chromosomes. In some embodiments, additional chromosomal homologues are selected at least partially based on the genetic characteristics of one or more chromosomal homologues (and/or chromosomes) pre-existing in a cell to which the chromosomal homologues are to be added.

In illustrative embodiments, one or more chromosomal homologues are selected according to their genetic characteristics. Subsequent chromosomal homologues are then selected at least partially based on the already-selected genetic characteristics. For example, in order to prevent disease, or in order to obtain complementary traits, and/or because gene systems are involved in a disease or a trait and the genes are optionally located on multiple chromosomes. In illustrative embodiments, selection of additional chromosomal homologues is at least partially based on one or more alleles of one or more independent genes located on a previously selected chromosome and/or one or more alleles of one or more gene systems located on a previously selected chromosome. In some embodiments, one or more additional genes that are part of a gene system are located on the additional chromosome as well as one or more already-selected chromosome.

In some embodiments, selecting one or more chromosomal homologues at least partially based on one or more genetic characteristics of the one or more homologues includes selecting the one or more chromosomal homologues at least partially based on one or more genetic characteristics of one or more reference chromosomes and/or one or more target chromosomes. In illustrative embodiments, one or more reference and/or target genetic characteristics for one or more chromosomal homologues has been identified, and chromosomal homologues are selected based on the presence and/or absence of one or more of these genetic characteristics.

In some embodiments, selecting one or more chromosomal homologues at least partially based on one or more genetic characteristics of the one or more homologues includes selecting the one or more chromosomal homologues at least partially based on one or more genetic characteristics of one or more germ line cells. The germ line cells are optionally the germ line cells to which the one or more chromosomal homologues are destined to be provided (e.g. stem cells, oocytes, spermatogonia, etc.), one or more related germ line cells (e.g. related polar bodies, related spermatids, and/or related stem cells), and/or the germ line cells intended to be used for fertilization (e.g. second germ line cells optionally from a different donor (e.g. stem cells, oocytes, spermatozoa, related polar bodies, related spermatids, etc.)).

In some embodiments, selecting one or more chromosomal homologues at least partially based on one or more genetic characteristics of the one or more homologues includes selecting the one or more chromosomal homologues at least partially based on one or more genetic characteristics of one or more related somatic cells. In illustrative embodiments, the one or more related somatic cells are the cells in which the one or more chromosomal homologues are provided in the process of providing the chromosomal homologues to germ cells. In illustrative embodiments, the one or more related somatic cells are related to (from the same donor as) the cells with which the germ line cells with the selected chromosomal homologues are optionally fertilized and/or combined.

In illustrative embodiments, selection of one or more chromosomal homologues is at least partially based on the genetic characteristics of the chromosomal homologues. The genetic characteristics of the chromosomal homologues may be provided, sent, and/or obtained electronically, optionally from a database, for example. The genetic characteristics of the chromosomal homologues may be determined directly, optionally through destructive (e.g. if there are multiple copies and/or the ability to obtain additional copies) and/or non-destructive means (e.g. if the copy identified will be the copy provided to the germ line cell). The genetic characteristics of the chromosomal homologues may be determined based on knowledge (and/or determination) of the genetic characteristics of the chromosomes in other cells (e.g. subtractively based on the determination of genetic characteristics of one or more related polar bodies or one or more related spermatids). Methods for performing these methods are known in the art and/or described herein.

In some embodiments, selecting one or more chromosomal homologues at least partially based on one or more genetic characteristics includes selecting the one or more chromosomal homologues at least partially based on one or more genetic characteristics of mitochondrial DNA. In some embodiments, the mitochondrial DNA is the mitochondrial DNA in the one or more germ line cells either that the chromosomal homologues will be provided to, or the mitochondrial DNA in the germ line cells to be used for fertilization. In some embodiments, the mitochondrial DNA is the mitochondrial DNA of related somatic cells of the either of the germ line cells (e.g. somatic cells of the same donor). In some embodiments, the mitochondrial DNA is the mitochondrial DNA of somatic cells of one or more relatives of one or more of the germ cell donors.

In some embodiments, one or more methods include providing and/or co-localizing the one or more germ line cells to and/or with the one or more second germ line cells for fertilization.

In some embodiments, one or more of these methods may be used for optimizing one or more germ cells. In illustrative embodiments, one or more germ cells may be optimized by selecting and providing to the germ cell one or more chromosomal homologues at least partially based on one or more genetic characteristics. The genetic characteristics are optionally selected for or against, optionally based on one or more reference chromosomes. For example, chromosomal homologues with haplotypes associated with one or more diseases would be selected against. Chromosomal homologues with haplotypes associated with one or more positive traits depending on the animal species would be selected for. A weighting of the chromosomes based on selection against and/or selection for would optionally be used to determine the chromosomal homologue selected.

In some embodiments, one or more of these methods may be used for treating, ameliorating, and/or preventing one or more genetic diseases. In illustrative embodiments, one or more chromosomal homologues associated with one or more diseases and/or disorders may be identified in a germ cell line. The germ cell line is optionally treated to genetically remove the chromosomal homologue associated with the disease and/or disorder. Another chromosomal homologue not associated with the disease and/or disorder is then identified and provided to the germ cell line.

In some embodiments, one or more methods include selecting one or more variants of one or more mitochondrial chromosomes at least partially based on one or more genetic characteristics of the one or more variants; and optionally providing the selected one or more mitochondrial chromosome variants to one or more (e.g. first) germ line cells. Methods, apparatus, and/or systems described for one or more other embodiments of the invention are also applicable to this embodiment of the invention unless in conflict.

In some embodiments, one or more methods optionally include obtaining one or more mitochondrial chromosome variants, optionally from one or more (e.g. second) germ line cells. In some embodiments, one or more methods optionally include removing one or more mitochondrial chromosome variants, optionally from one or more (e.g. first) germ line cells. In some embodiments, one or more methods optionally include providing and/or co-localizing one or more (e.g. first) germ line cells to and/or with one or more (e.g. third) germ line cells optionally for fertilization. In some embodiments, one or more of the one or more germ line cells may be designated as first, second, third, and/or fourth germ line cells, for example. In some embodiments, the first, second, third, and/or fourth germ line cells are different germ line cells, unless one or more are indicated as optionally the same germ line cells.

In some embodiments, the one or more mitochondrial chromosome variants may include one or more recombinant and/or genetically modified mitochondrial chromosome variants. In illustrative embodiments, one or more mitochondrial chromosomes from a donor may be modified optionally to remove one or more gene and/or allele, and optionally to replace the one or more gene and/or allele with an alternate version (e.g. if the gene and/or allele was mutated and/or associated with a disease/disorder). In illustrative embodiments, one or more mitochondrial chromosomes from a donor may be modified optionally to insert one or more gene and/or allele, optionally to provide a benefit to a possible subsequent biological entity (e.g. increased metabolism, increased energy, increased stamina, etc.).

In some embodiments, the one or more mitochondrial chromosome variants may include one or more mitochondrial chromosome variants from more than one biological entity. In some embodiments, the one or more mitochondrial chromosome variants may include one or more first mitochondrial chromosome variants, one or more second mitochondrial chromosome variants, one or more third mitochondrial chromosome variants, one or more fourth mitochondrial chromosome variants, and/or one or more fifth mitochondrial chromosome variants, etc. In illustrative embodiments, one or more mitochondrial chromosome variants are selected from several biological entities of the same species, and/or of different species. Selection of one or more mitochondrial chromosome variants from several biological entities of one species optionally enhances the chance of compatibility with the nuclear genome and/or the chance for enhanced mitochondrial performance.

In some embodiments, one or more methods include sorting and/or separating one or more mitochondrial chromosome variants at least partially based on one or more genetic characteristics of the one or more mitochondrial chromosome variants. In some embodiments, one or more methods include sorting and/or separating one or more mitochondrial chromosome variants optionally from one or more non-selected mitochondrial chromosome variants and/or from one or more mitochondria and/or mitochondrial components, and/or nuclear genomes and/or chromosomes.

In illustrative embodiments, the one or more mitochondrial chromosome variants are sorted and/or selected from other non-selected mitochondrial chromosome variants by detection/identification of the variants while the variants are present in mitochondria and/or cells, optionally through mitochondria and/or cell sorting. In illustrative embodiments, the one or more mitochondrial chromosome variants are sorted and/or separated from non-selected variants optionally following isolation from mitochondria and/or cells. Methods for separating/sorting are known in the art and/or described herein.

In some embodiments, one or more methods include providing and/or co-localizing one or more mitochondrial chromosome variants to and/or with one or more germ line cells (e.g. stem cells, oocytes, and/or ova) and/or one or more somatic cells (e.g. stem cells, pluripotent cells, totipotent cells, and/or replicating cells). In some embodiments, the germ line cells are enucleated. In some embodiments, the germ line cells and/or the somatic cells have optionally a reduced amount, very few, and/or no endogenous mitochondria and/or mitochondrial chromosomes.

As used herein, the term “no endogenous mitochondria and/or mitochondrial chromosomes” may include an amount below the limits of detection, an amount that is considered not statistically significant, and/or an amount that is not scientifically and/or commercially reasonable to remove. As used herein, the term “reduced amount” includes any amount that is below the standard level for cells of that types, below the level of other cells of that type from the donor, and/or below the level of that cell prior to treatment to reduce the amount. As used herein, the term “below” refers to a statistically significant reduction and/or a scientifically detectable reduction. Numbers of mitochondria and/or mitochondrial chromosomes in cells and methods for the reduction in numbers are known in the art and/or described herein.

In illustrative embodiments, one or more mitochondrial chromosome variants are provided to one or more stem cells that optionally have had their endogenous mitochondrial chromosome variants removed. The stem cells are optionally proliferated, and then differentiated into oocytes and/or ova, optionally in preparation for fertilization. In illustrative embodiments, one or more mitochondrial chromosome variants are provided to one or more somatic cells, optionally replicating somatic cells and/or somatic stem cells, for maintenance and/or replication. In illustrative embodiments, the stem cells are subsequently differentiated into germ line cells.

In some embodiments, one or more methods include obtaining one or more variants of mitochondrial chromosomes, optionally from one or more germ line cells and/or one or more somatic cells. In some embodiments, obtaining one or more variants includes identifying, purchasing, isolating, extracting, synthesizing, removing, and/or replicating one or more mitochondrial chromosome variants. In some embodiments, the one or more germ line cells include, but are not limited to, one or more haploid germ cells (e.g. spermatozoa, spermatocytes, spermatids, ova), one or more diploid germ cells, one or more stem cells (e.g. one or more spermatogonia and/or oogonia), and/or one or more oocytes (e.g. primary and/or secondary). In some embodiments, the one or more somatic cells, include, but are not limited to, one or more stem cells (e.g. embryonic, cord blood, fetal, hematopoietic, mesenchymal, epithelial, among others), one or more progenitor cells, and/or one or more lymphocytes.

In illustrative embodiments, one or more mitochondrial chromosome variants may initially be identified in one or more cells during non-destructive cell sorting, for example. Depending on the type of cells, they may be encouraged to proliferate (e.g. stem cells), or the mitochondria (and/or mitochondrial chromosomes) may be transferred to other cells for maintenance and/or proliferation. In illustrative embodiments, methods for obtaining one or more variants of mitochondrial chromosomes include isolating one or more mitochondria from on or more cells, isolating one or more mitochondrial chromosomes from one or more cells, and/or. genetically manipulating one or more mitochondrial chromosomes. Methods for obtaining one or more mitochondrial chromosomes are known in the art and/or described herein.

In some embodiments, one or more methods include selecting one or more variants of mitochondrial chromosomes at least partially based on one or more genetic characteristics of the one or more variants. In some embodiments, the one or more genetic characteristics of the one or more variants include, but are not limited to, one or more alleles of one or more genes of the one or more mitochondrial chromosome variants.

In some embodiments, the one or more methods include removing, isolating, extracting, and/or eliminating one or more mitochondria and/or mitochondrial chromosome variants from one or more germ cell lines and/or somatic cells. In some embodiments, the one or more variants are removed, isolated, extracted, and/or eliminated from one or more stem cells, proliferating cells, and/or somatic cells, among others. In illustrative embodiments, one or more chromosomal variants (and/or mitochondria) are optionally removed from one or more cells in order to facilitate the re-insertion of one or more selected chromosomal variants and/or mitochondria. In illustrative embodiments, individual chromosomal variants are optionally removed and/or intact (or partially intact mitochondria) may be removed. Methods for removing, eliminating, extracting, and/or isolating one or more mitochondria and/or mitochondrial chromosome variants are known in the art and/or described herein.

In some embodiments, one or more methods include selecting one or more variants of mitochondrial chromosomes at least partially based on one or more genetic characteristics of the one or more variants. Genetic characteristics and methods for selection based on one or more genetic characteristics are known in the art and described herein. Methods described for other embodiments herein are specifically intended as applicable to this embodiment, to the extent that such methods are not inconsistent herewith. In some embodiments, selecting one or more chromosome variants at least partially based on one or more genetic characteristics of the one or more variants includes identifying and/or analyzing one or more variants at least partially based on one or more genetic characteristics. In some embodiments, selection is at least partially based on one or more alleles of one or more genes of the one or more variants.

In some embodiments, the one or more mitochondrial chromosome variants are at least partially selected based on one or more genetic characteristics of one or more reference and/or target mitochondrial chromosomes. In illustrative embodiments, one or more reference mitochondrial genomes may include one or more genomes that are not known to be associated with a genetic disease, and/or that are associated with one or more traits that may be considered desirable. In illustrative embodiments, one or more target mitochondrial genomes may include one or more genomes that are optionally specifically tailored to be genetically compatible with and/or genetically similar to mitochondrial genomes of one or more of the nuclear genomes (e.g. of the one or more germ line cells).

In some embodiments, the one or more mitochondrial chromosome variants are at least partially selected based on one or more genetic characteristics of one or more female germ line cells, optionally including one or more genetic characteristics of one or more mitochondrial chromosome variants (and/or one or more nuclear chromosome homologues) of the one or more female germ line cells. In illustrative embodiments, selection of one or more mitochondrial chromosome variants is at least partially based on, for example, the mitochondrial variants of the cell (optionally a stem cell, ova, and/or oocyte) to which the mitochondria are to be provided. In the event that the nucleus of that cell is to be maintained (and not enucleated), the selection of one or more mitochondrial chromosome variants is optionally at least partially based on the genetic characteristics of the nuclear genome of that cell. In the event that the cell is to be enucleated (or has been enucleated), selection of one or more mitochondrial chromosome variants is optionally at least partially based on one or more genetic characteristics of the nuclear genome of the nucleus to be provided to the cell (or already provided to the cell).

In some embodiments, the one or more mitochondrial chromosome variants are at least partially selected based on one or more genetic characteristics of one or more somatic cells, optionally somatic stem cells. In some embodiments, the one or more somatic cells and the one or more female germ line cells are from a single donor. In some embodiments, the somatic stem cells are differentiated to germ line cells. In some embodiments, the one or more genetic characteristics of the one or more somatic cells include one or more genetic characteristics of one or more mitochondrial genomes and/or one or more nuclear genomes of the one or more somatic cells.

In illustrative embodiments, the one or more mitochondrial chromosome variants are selected at least partially based on one or more genetic characteristics (e.g. mitochondrial genome and/or nuclear genome) of the one or more female germ cell to which they will be provided. The genetic characteristics of the one or more female germ cell to which they will be provided may be determined from the cell itself, from other related germ line cells (e.g. those from the same donor), and/or from related somatic cells (e.g. those from the same donor). In some cases, the mitochondrial genomes may be inferred based on the genetic characteristics of mitochondria from relatives of the donor, for example a mother, sisters, and/or aunts and cousins. The somatic cells are optionally from any easily obtained source, such as, but not limited to, blood, skin, mucosal surfaces, etc.

In some embodiments, the one or more mitochondrial chromosome variants are at least partially selected based on one or more genetic characteristics (e.g. nuclear genomes and/or mitochondrial genomes) of one or more male germ line cells, optionally one or more stem cells (e.g. germ line and/or somatic), one or more diploid germ line cells, and/or one or more haploid germ cells, optionally one or more spermatozoa, one or more spermatids, and/or one or more spermatocytes. In some embodiments, the male germ line cells are optionally the male germ line cells intended to be used during fertilization.

In some embodiments, one or more methods include providing and/or co-localizing the one or more male germ line cells (and/or one or more male germ line nuclei/genomes) to and/or with the one or more female germ line cells for fertilization. In illustrative embodiments, the one or more mitochondrial chromosome variants are destined to be provided to one or more female germ line cells (e.g. oocytes), that are intended to be fertilized by a male germ line haploid genome. In this instance, for example, the mitochondria are optionally selected at least partially based on one or more genetic characteristics of the male germ line genome (e.g. nuclear genome characteristics, and optionally mitochondrial chromosome characteristics).

In some embodiments, more than one mitochondrial chromosome variant may be selected, optionally designated first, second, third, fourth, fifth, etc. These additional mitochondrial chromosome variants may be selected based on one or more genetic characteristics optionally of their own genetic sequence, for example, but also optionally based on one or more genetic characteristics of the one or more (e.g. previously) selected mitochondrial chromosome variants, optionally one or more alleles of one or more genes of the one or more (previously) selected mitochondrial chromosome variants. In illustrative embodiments, a selection of mitochondrial chromosome variants is optionally selected with the genetic characteristics of each variants selected at least partially dependent on the genetic characteristics of the other variants selected.

In some embodiments, one or more of these methods may be used for optimizing one or more germ cells, optionally one or more oocytes. In illustrative embodiments, one or more female germ line cells (and/or somatic stem cells) is optionally treated to incapacitate, inactivate and/or remove a majority of endogenous mitochondria and/or mitochondrial chromosomes. Subsequently (and/or concurrently) selected mitochondrial chromosomes (optionally within mitochondria) are provided to the cell. The cells is then differentiated to an oocyte and/or an ova for fertilization, as necessary.

In some embodiments, one or more of these methods may be used for treating, ameliorating, and/or preventing one or more genetic diseases and/or disorders. In some embodiments, one or more unselected mitochondrial chromosome variants have been associated with the one or more genetic diseases, and/or one or more selected mitochondrial chromosome variants have been not been associated with one or more diseases and/or disorders. In some embodiments, a combination of one or more nuclear genomes and one or more unselected mitochondrial chromosome variants have been associated with the one or more genetic diseases, and/or a combination of one or more nuclear genomes and one or more selected mitochondrial chromosome variants have been not been associated with one or more diseases and/or disorders.

Embodiments of one or more methods include optionally providing one or more female nuclei to one or more enucleated male germ line cells to obtain one or more female-nucleated male germ line cells; optionally proliferating the one or more female-nucleated male germ line cells; optionally inducing spermatogenesis in the one or more female-nucleated male germ line cells; and selecting one or more of the one or more female-nucleated male germ line cells at least partially based on one or more genetic characteristics. Methods, apparatus, and/or systems described for one or more other embodiments of the invention are also applicable to this embodiment of the invention to the extent that such methods, apparatus, and/or systems are not in conflict herewith.

As sued herein, the term “female-nucleated male germ line cells” includes male germ line cells (e.g. stem cells, spermatocytes, spermatids, spermatozoa) wherein the nuclear genome is from a female donor. In illustrative embodiments, the female genome is transferred into an enucleated male germ line cell. In illustrative embodiments, a female donor cell, optionally a female donor stem cell, is differentiated to a male germ line cell. In illustrative embodiments, the female genome may be at least partially modified, optionally by chromosomal transfer/removal (e.g. of a Y/X chromosome, and/or autosomal chromosomal homologues, optionally recombinant chromosomes).

In some embodiments, the one or more female nuclei are from one or more germ line cells and/or from one or more somatic cells. In some embodiments, the one or more germ line cells include, but are not limited to, one or more haploid germ cells, one or more diploid germ cells, one or more ova, one or more oocytes (e.g. primary and/or secondary), one or more polar bodies, one or more stem cells. In some embodiments, the one or more somatic cells include, but are not limited to, one or more stem cells and/or one or more progenitor cells. In some embodiments, the one or more somatic cells are one or more easily obtained cells from tissues including, for example, but not limited to, blood, skin, hair follicles, and/or mucosal tissue.

In some embodiments, one or more methods optionally include providing one or more female nuclei to one or more enucleated male germ line cells optionally including, but not limited to, stem cells. In illustrative embodiments, the one or more female nuclei are optionally provided to (or optionally are present in) one or more stem cells, optionally including, but not limited to, one or more fetal stem cells, embryonic stem cells, germ line stem cells, and/or somatic stem cells (e.g. hematopoietic, mesenchymal and/or epithelial stem cells) which are then differentiated to male germ line cells, optionally male germ line stem cells. In illustrative embodiments, the one or more female nuclei are optionally provided to one or more male germ line stem cells, optionally spermatogonia. Such methods are known in the art and/or described herein.

In some embodiments, the one or more methods optionally include proliferating the one or more female-nucleated male germ line cells optionally in vitro, in situ and/or in vivo. In illustrative embodiments, female genomes are provided to (or optionally are present in) one or more stem cells. Optionally the stem cells are amplified to increase the copy number, optionally before differentiation into female-nucleated male germ line stem cells. Optionally the stem cells are differentiated to male germ line stem cells prior to proliferation of the female-nucleated male germ line stem cells.

In some embodiments, the one or more methods optionally include inducing spermatogenesis in the one or more female-nucleated male germ line cells optionally in vitro, in situ and/or in vivo. In illustrative embodiments, proliferated female nucleated male germ line cells are induced to undergo spermatogenesis, optionally to increase the genetic diversity of the female-nucleated male germ line cells. Methods for inducing spermatogenesis are known in the art and/or described herein.

In some embodiments, one or methods include selecting one or more of the one or more female-nucleated male germ line cells at least partially based on one or more genetic characteristics. Genetic characteristics and methods for selection based on one or more genetic characteristics are known in the art and described herein. Methods described for other embodiments herein are specifically intended as applicable to this embodiment, to the extent that such methods are not inconsistent herewith.

In some embodiments, the selection of one or more female-nucleated male germ line cells is at least partially based on one or more genetic characteristics one or more reference and/or target genomes, optionally nuclear genomes and/or mitochondrial genomes. In some embodiments, the selection of one or more female-nucleated male germ line cells is at least partially based on one or more genetic characteristics one or more chromosomes.

In some embodiments, one or methods include selecting one or more of the one or more female-nucleated male germ line cells at least partially based on one or more genetic characteristics (optionally of the nuclear genome and/or one or more chromosomal homologues) of one or more female germ line cells (e.g. intended for fertilization and/or related genomes). In some embodiments, one or more genetic characteristics of the one or more female germ line cells include one or more genetic characteristics of one or more mitochondrial chromosome variants of the one or more female germ line cells. In some embodiments, the one or more female germ line cells are one or more stem cells, one or more oocytes, one or more ova, and/or one or more polar bodies.

In some embodiments, one or methods include selecting one or more of the one or more female-nucleated male germ line cells at least partially based on one or more genetic characteristics (optionally of the nuclear genome and/or one or more chromosomal homologues) of one or more male germ line cells (e.g. male germ line genomes that will be used for fertilization and/or related genomes). In some embodiments, one or more male germ line cells are one or more spermatozoa, one or more related spermatids, one or more spermatocytes, and/or one or more stem cells. In some embodiments, the one or more stem cells and the one or more male germ line cells are from a single donor.

In some embodiments, one or more methods include providing the one or more female-nucleated male germ cells to one or more female germ line cells (e.g. one or more stem cells, one or more oocytes and/or ova) optionally for fertilization. In some embodiments, fertilization is optionally through a male germ line genome or optionally a female germ line genome. In some embodiments, one or more methods include providing the one or more female-nucleated male germ cells (e.g. genomes) to one or more enucleated stem cells, oocytes and/or ova, optionally for fertilization. In some embodiments, fertilization is optionally through a male germ line genome or optionally a female germ line genome.

In some embodiments, one or more methods include optimizing female haploid genomes. In illustrative embodiments, one or more female genomes may be optimized by allowing a female nucleus to undergo spermatogenesis followed by selection for one or more genetic characteristics as described herein.

In one aspect, the disclosure is drawn to one or more compositions comprising one or more germ line genomes. In some embodiments, one or more compositions are generated using one or more of the methods described herein and/or one or more of the apparatus described herein, and/or one of the systems described herein.

In some embodiments, one or more compositions include one or more containers including one or more male germ line haploid genomes, the one or more male germ line haploid genomes selected at least partially based on one or more genetic characteristics of the one or more male germ line haploid genomes, the one or more genetic characteristics of the one or more male germ line haploid genomes selected at least partially based on one or more genetic characteristics of one or more female germ line genomes.

In some embodiments, one or more compositions include one or more containers including one or more at least partially decondensed male germ line haploid genomes, the one or more male germ line haploid genomes selected at least partially based on one or more genetic characteristics of the one or more male germ line haploid genomes. In some embodiments, one or more male germ line haploid genomes are selected at least partially based on one or more genetic characteristics of the one or more male germ line haploid genomes, the one or more genetic characteristics of the one or more male germ line haploid genomes selected at least partially based on one or more genetic characteristics of one or more female germ line genomes. In some embodiments, one or more of the at least partially decondensed male germ line haploid genomes is at least partially recondensed.

In some embodiments, one or more compositions include one or more containers including one or more related spermatid genomes, the one or more male germ line haploid genomes selected at least partially based on one or more genetic characteristics of one or more related spermatid genomes. In some embodiments, the one or more related spermatid genomes are selected at least partially based on one or more of the one or more genetic characteristics of the one or more related spermatid genomes, the one or more genetic characteristics of the one or more related spermatid genomes selected at least partially based on one or more genetic characteristics of one or more female germ line genomes.

In some embodiments, one or more compositions include one or more containers including one or more female germ line haploid genomes, the one or more female germ line haploid genomes selected at least partially based on one or more genetic characteristics of one or more related polar body genomes. In some embodiments, the one or more female germ line haploid genomes are selected at least partially based on the one or more genetic characteristics of the one or more related polar body genomes, the one or more genetic characteristics of the one or more related polar body genomes selected at least partially based on one or more genetic characteristics of one or more male germ line genomes. In some embodiments, the one or more male germ line genomes are one or more male haploid germ line genomes.

In some embodiments, one or more compositions include one or more containers including one or more female germ line haploid genomes, the one or more female germ line haploid genomes selected at least partially based on one or more genetic characteristics of one or more male germ line genomes. In some embodiments, the one or more male germ line genomes are one or more male haploid germ line genomes.

In some embodiments, one or more compositions include one or more containers including one or more optimized germ line cells, the optimized germ cells having one or more homologues of one or more chromosomes individually selected at least partially based on one or more genetic characteristics of the one or more chromosomal homologues.

In some embodiments, one or more compositions include one or more containers including one or more optimized germ cells, the optimized germ cells having one or more variants of one or more mitochondrial chromosomes selected based on one or more genetic characteristics of the one or more variants.

In some embodiments, one or more compositions include one or more containers including one or more optimized female-nucleated male germ line cells, the optimized female-nucleated male germ line cells selected at least partially based on one or more genetic characteristics.

In one aspect, the disclosure is drawn to one or more apparatus for selecting one or more germ line genomes, one or more chromosomal homologues, and/or one or more mitochondrial variants at least partially based on one or more genetic characteristics of one or more germ line genomes, one or more chromosomal homologues, and/or one or more mitochondrial variants. In some embodiments, one or more of the methods described herein may be performed on one or more apparatus. In some embodiments, one or more of the compositions described herein may be created using one or more apparatus. In some embodiments, one or more system methods may be performed on one or more apparatus, and/or one or more apparatus may include one or more system or computing devices described herein. Although generally discussed in light of chromosomal genomes, the apparatus and methods described are also applicable to chromosomal and mitochondrial chromosome selection unless in conflict.

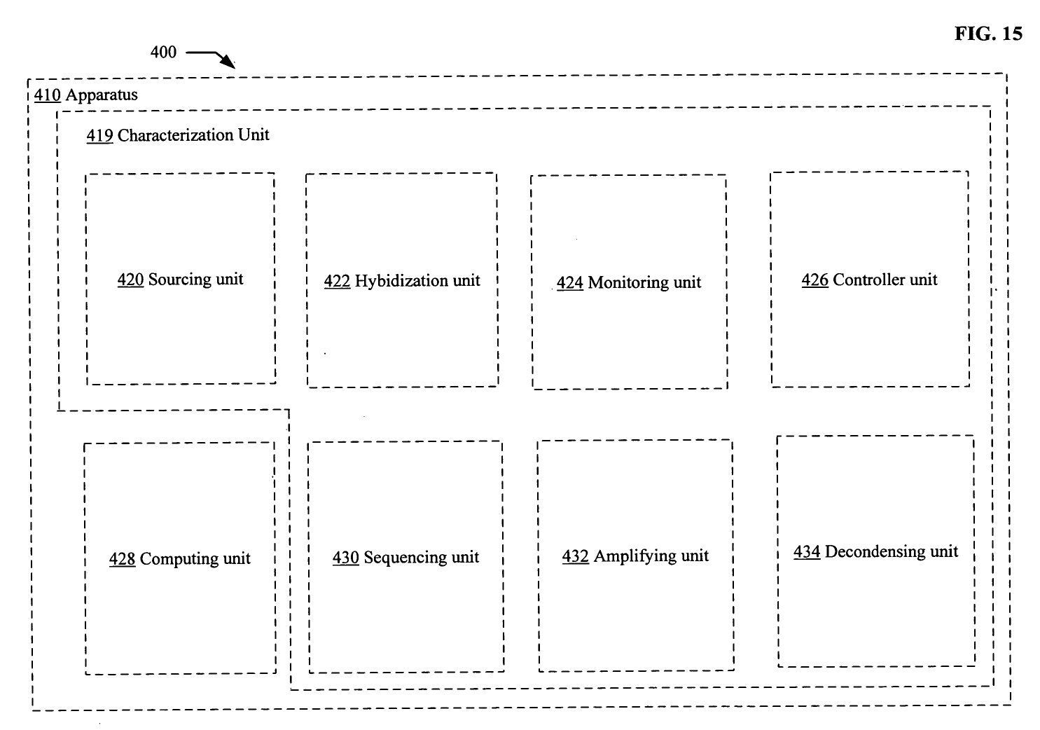

FIG. 15 shows a schematic 400 of an illustrative apparatus 410 in which embodiments may be implemented. The apparatus 410 is optionally operable for characterizing, monitoring, detecting, hybridizing, amplifying, sequencing, identifying, analyzing, and/or determining one or more genetic characteristics of one or more germ line genomes, as well as optionally selecting, separating, sorting, providing, and/or co-localizing one or more germ line genomes. The apparatus may optionally be, or include, one or more units including, but not limited to, one or more characterization units 419, one or more sourcing units 420, one or more hybridization units 422, one or more monitoring units 424, one or more controller units 426, one or more computing units 428, one or more sequencing units 430, one or more amplifying units 432, and/or one or more decondensing units 434. In some embodiments, one or more of the units may be internal or external to the apparatus. In some embodiments, one or more of the units may be part of or separate from the apparatus.

In some embodiments, one or more characterization units 419 are operable to characterize one or more genetic characteristics of one or more genomes. In some embodiments, one or more characterization units 419 include and/or are the same as, one or more of one or more sourcing units 420, one or more hybridization units 422, one or more monitoring units 424, one or more controller units 426, one or more computing units 428, one or more sequencing units 430, one or more amplifying units 432, and/or one or more decondensing units 434.

In some embodiments, one or more apparatus 410 further includes one or more fluid flows. In some embodiments, the one or more fluid flows connect and/or allow the transfer of one or more germ line genomes as well as other components, including but not limited to probes and molecular markers, among one or more of the optional one or more units of the apparatus 410. In some embodiments, the one or more fluid flows are operable to provide, co-localize, remove and/or separate, optionally sequentially, one or more germ line genomes as well as other components. In some embodiments, the one or more fluid flows are operable to provide, co-localize, remove and/or separate, optionally sequentially, one or more germ line genomes as well as other components at one or more identifiable time intervals.

In some embodiments, one or more apparatus 410 includes one or more sourcing units 420 including one or more first sources of one or more male germ line haploid genomes and one or more second sources of one or more probes; one or more hybridization units 422 operable to co-localize one or more of the one or more probes with one or more nucleic acids of the one or more male germ line haploid genomes; one or more monitoring units 424 operable to detect one or more of the one or more probes hybridized to the one or more nucleic acids of the one or more male germ line haploid genomes; and one or more controller units 426 operable to select, sort, and/or separate one or more of the one or more male germ line haploid genomes at least partially based on the detection of one or more of the one or more probes hybridized to the one or more nucleic acids.

In some embodiments, one or more apparatus 410 includes one or more sourcing units 420 including one or more first sources of one or more male germ line haploid genomes and one or more second sources of one or more probes; one or more hybridization units 422 operable to co-localize one or more of the one or more probes with one or more nucleic acids of the one or more male germ line haploid genomes; one or more monitoring units 424 operable to detect one or more of the one or more probes hybridized to the one or more nucleic acids of the one or more male germ line haploid genomes; and one or more computing units 428 operable to determine the one or more male germ line haploid genomes to select, sort, and/or separate at least partially based on the detection of one or more of the one or more probes hybridized to the one or more nucleic acids.

In some embodiments, one or more apparatus 410 includes one or more first sources of one or more male germ line haploid genomes; one or more second sources of one or more probes; one or more monitors for detecting one or more of the one or more probes; one or more units for hybridizing one or more of the one or more probes with one or more nucleic acids of the one or more male germ line haploid genomes; and one or more controllers for selecting one or more of the one or more male germ line haploid genomes at least partially based on the detection of one or more of the one or more probes hybridized to the one or more nucleic acids.

In some embodiments, one or more apparatus 410 includes one or more detecting units operable to identify one or more genetic characteristics of one or more male germ line haploid genomes using one or more nucleic acid detecting molecules other than a polyamide or Hoechst; one or more first sourcing units containing one or more sources of one or more male germ line haploid genomes; one or more second sourcing units containing one or more sources of the one or more nucleic acid detecting molecules; and one or more first controller units operable to select one or more of the one or more male germ line haploid genomes at least partially based on the one or more genetic characteristics of the one or more male germ line haploid genomes.

In some embodiments, one or more apparatus includes one or more characterization units operable to detect and/or identify one or more probes hybridized to one or more nucleic acid sequences of one or more male germ line haploid genomes; and one or more controller units operable to select, sort, and/or separate one or more of the one or more male germ line haploid genomes at least partially based on the detection and/or identification of one or more probes hybridized to one or more nucleic acid sequences. In some embodiments, one or more apparatus includes one or more characterization units operable to detect and/or identify one or more probes hybridized to one or more nucleic acid sequences of one or more male germ line haploid genomes; and one or more computing units operable to determine the one or more male germ line haploid genomes to select, sort, and/or separate at least partially based on the detection and/or identification of one or more probes hybridized to one or more nucleic acid sequences.

In some embodiments, one or more apparatus 410 includes one or more sourcing units 420 including one or more first sources of one or more male germ line haploid genomes; one or more monitoring units 424 operable to detect one or more genetic characteristics of the one or more male germ line haploid genomes; one or more computing units 428 operable to receive one or more inputs, the one or more inputs including data representative of one or more genetic characteristics of one or more female germ line genomes; one or more controller units 426 operable to select, sort, and/or separate one or more of the one or more male germ line haploid genomes at least partially based on the one or more genetic characteristics of the one or more female germ line genomes. In some embodiments, one or more apparatus 410 includes one or more sourcing units 420 including one or more first sources of one or more male germ line haploid genomes; one or more monitoring units 424 operable to detect one or more genetic characteristics of the one or more male germ line haploid genomes; one or more computing units 428 operable to receive one or more inputs, the one or more inputs including data representative of one or more genetic characteristics of one or more female germ line genomes; and operable to determine the one or more male germ line haploid genomes to select, sort, and/or separate at least partially based on the one or more genetic characteristics of the one or more female germ line genomes.

In some embodiments, one or more apparatus 410 includes one or more first sources of one or more male germ line haploid genomes; one or more monitors for detecting one or more genetic characteristics of the one or more male germ line haploid genomes; one or more units for receiving one or more inputs, the one or more inputs including data representative of one or more genetic characteristics of one or more female germ line genomes; one or more controllers for selecting one or more of the one or more male germ line haploid genomes at least partially based on the one or more genetic characteristics of the one or more female germ line genomes.

In some embodiments, one or more apparatus 410 includes one or more characterization units 419 operable to detect and/or identify one or more genetic characteristics of one or more male germ line haploid genomes; one or more computing units 428 operable to receive one or more inputs, the one or more inputs including data representative of one or more genetic characteristics of one or more female germ line genomes; and one or more controller units 426 operable to select, sort, and/or separate one or more of the one or more male germ line haploid genomes at least partially based on the one or more genetic characteristics of the one or more female germ line genomes. In some embodiments, one or more apparatus 410 includes one or more characterization units 419 operable to detect and/or identify one or more genetic characteristics of one or more male germ line haploid genomes; one or more computing units 428 operable to receive one or more inputs, the one or more inputs including data representative of one or more genetic characteristics of one or more female germ line genomes; and operable to determine the one or more male germ line haploid genomes to select, sort, and/or separate at least partially based on the one or more genetic characteristics of the one or more female germ line genomes.

In some embodiments, one or more apparatus 410 includes one or more computing units 428 operable to receive one or more inputs, the one or more inputs including data representative of one or more genetic characteristics of one or more female germ line genomes; and one or more controller units 426 operable to select, sort, and/or separate one or more of the one or more male germ line haploid genomes at least partially based on the one or more genetic characteristics of the one or more female germ line genomes. In some embodiments, one or more apparatus 410 includes one or more computing units 428 operable to receive one or more inputs, the one or more inputs including data representative of one or more genetic characteristics of one or more female germ line genomes; and operable to determine the one or more male germ line haploid genomes to select, sort, and/or separate at least partially based on the one or more genetic characteristics of the one or more female germ line genomes.

In some embodiments, one or more apparatus 410 includes one or more sourcing units 420 including one or more first sources of one or more male germ line haploid genomes, the one or more male germ line haploid genomes at least partially condensed; one or more decondensing units 434 operable to at least partially or completely decondense the one or more male germ line haploid genomes; one or more monitoring units 424 operable to detect one or more genetic characteristics of the one or more male germ line haploid genomes; and one or more controller units 426 operable to select one or more of the one or more male germ line haploid genomes at least partially based on the one or more genetic characteristics of the one or more male germ line haploid genomes. In some embodiments, one or more apparatus 411 includes one or more first sources of one or more male germ line haploid genomes, the one or more male germ line haploid genomes at least partially condensed; one or more units for decondensing the one or more male germ line haploid genomes; one or more monitors for detecting one or more genetic characteristics of the one or more male germ line haploid genomes; and one or more controllers for selecting one or more of the one or more male germ line haploid genomes at least partially based on the one or more genetic characteristics of the one or more male germ line haploid genomes.

In some embodiments, one or more apparatus 410 includes one or more sourcing units 420 including one or more first sources of one or more related spermatid genomes; one or more monitoring units 424 operable to detect one or more genetic characteristics of one or more of the one or more related spermatid genomes; and one or more controller units 426 operable to select, sort, and/or separate one or more of the one or more related spermatid genomes at least partially based on one or more of the genetic characteristics of one or more of the one or more related spermatid genomes.

In some embodiments, one or more apparatus 410 includes one or more sourcing units 420 including one or more first sources of one or more related spermatid genomes; one or more computing units 428 operable to receive one or more inputs, the one or more inputs including data representative of one or more characteristics of one or more of the one or more related spermatid genomes; and one or more controller units 426 operable to select, sort, and/or separate one or more of the one or more related spermatid genomes at least partially based on the one or more genetic characteristics of one or more of the one or more related spermatid genomes.

In some embodiments, one or more apparatus 410 includes one or more sourcing units 420 including one or more first sources of one or more related spermatid genomes; one or more computing units 428 operable to receive one or more inputs, the one or more inputs including data representative of one or more characteristics of one or more of the one or more related spermatid genomes; and operable to determine the one or more related spermatid genomes to select, sort, and/or separate at least partially based on the one or more genetic characteristics of one or more of the one or more related spermatid genomes.

In some embodiments, one or more apparatus 410 includes one or more characterization units 419 operable to determine, detect, and/or identify one or more genetic characteristics of one or more related spermatid genomes; and one or more controller units 426 operable to select, sort, and/or separate one or more of the one or more related spermatid genomes at least partially based on the one or more genetic characteristics of one or more of the one or more related spermatid genomes. In some embodiments, one or more apparatus 410 includes one or more characterization units 419 operable to determine, detect, and/or identify one or more genetic characteristics of one or more related spermatid genomes; and one or more computing units 428 operable to determine the one or more related spermatid genomes to select, sort, and/or separate at least partially based on the one or more genetic characteristics of one or more of the one or more related spermatid genomes.

In some embodiments, one or more apparatus 410 includes one or more sourcing units 420 including one or more first sources of one or more related polar body genomes; one or more monitoring units 424 operable to detect one or more genetic characteristics of one or more of the one or more related polar body genomes; and one or more controller units 426 operable to select, sort, and/or separate one or more of the one or more related polar body genomes at least partially based on one or more of the genetic characteristics of one or more of the one or more related polar body genomes. In some embodiments, one or more apparatus 410 includes one or more sourcing units 420 including one or more first sources of one or more related polar body genomes; one or more computing units 428 operable to receive one or more inputs, the one or more inputs including data representative of one or more characteristics of one or more of the one or more related polar body genomes; and one or more controller units 426 operable to select, sort, and/or separate one or more of the one or more related polar body genomes at least partially based on the one or more genetic characteristics of one or more of the one or more related polar body genomes. In some embodiments, one or more apparatus 410 includes one or more sourcing units 420 including one or more first sources of one or more related polar body genomes; one or more computing units 428 operable to receive one or more inputs, the one or more inputs including data representative of one or more characteristics of one or more of the one or more related polar body genomes; and operable to determine the one or more related polar body genomes to select, sort, and/or separate at least partially based on the one or more genetic characteristics of one or more of the one or more related polar body genomes.

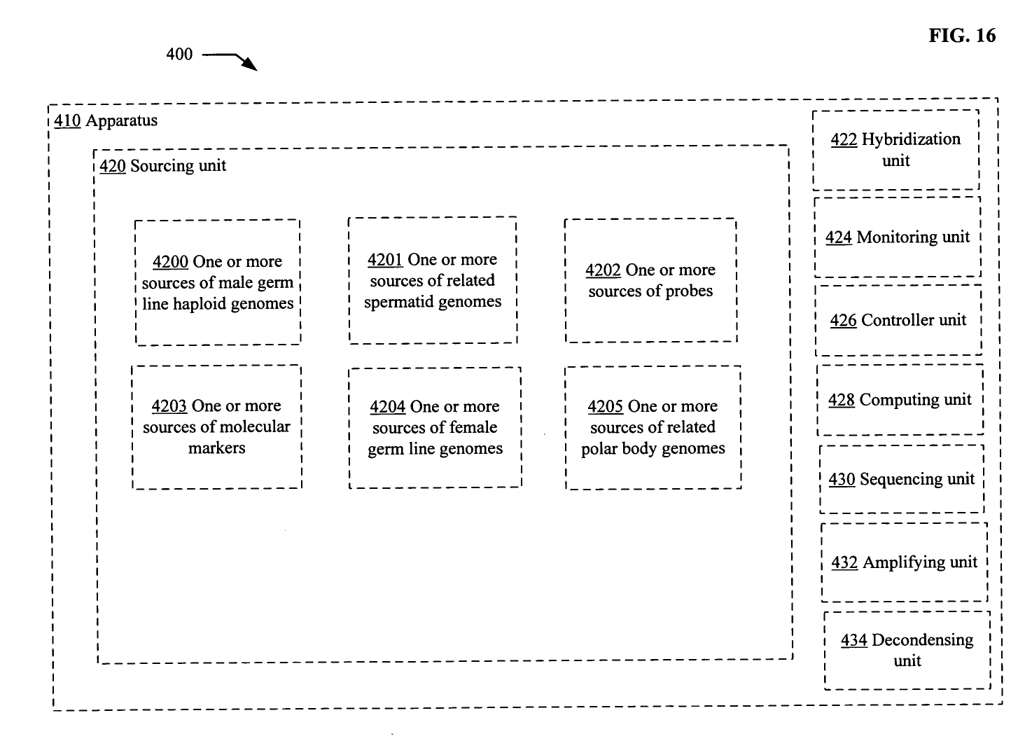

FIG. 16 shows a schematic 400 of illustrative embodiments of the optional apparatus 410 of FIG. 15, with specific illustrative embodiments of one or more sourcing units 420, including, but not limited to, unit 4200, unit 4201, unit 4202, unit 4203, unit 4204, and unit 4205. In some embodiments, one or more sourcing units 420 are internal to the apparatus 410; in some embodiments, one or more sourcing units are external to the apparatus 410. In some embodiments, one or more sourcing units are part of, the same as, and/or included in one or more characterization units 419, one or more of one or more hybridization units 422, one or more monitoring units 424, one or more controller units 426, one or more computing units 428, one or more sequencing units 430, one or more amplifying units 432, and/or one or more decondensing units 434.

In some embodiments, one or more sourcing units include one or more first sources of one or more male germ line haploid genomes 4200 and/or one or more related spermatid genomes 4201, the one or more first sources optionally positioned to provide the one or more male germ line haploid genomes and/or related spermatid genomes, to one or more first locations, one or more first units, one or more monitoring units, one or more controller units, one or more computing units, one or more sequencing units, and/or one or more hybridization units.

In some embodiments, one or more sourcing units 420 include one or more second sources of one or more probes 4202 and/or one or more molecular markers 4203, the one or more second sources optionally positioned to provide the one or more probes to one or more second locations, one or more first units, one or more monitoring units, one or more controller units, one or more computing units, one or more sequencing units, and/or one or more hybridization units.

In some embodiments, one or more sourcing units 420 include one or more third sources of one or more female germ line genomes 4204 and/or one or more related polar body genomes 4205, the one or more third sources optionally positioned to provide the one or more female germ line genomes and/or related polar body genomes to one or more third locations, one or more first units, one or more monitoring units, one or more controller units, one or more computing units, one or more sequencing units, and/or one or more hybridization units.

In some embodiments, one or more sourcing units 420 are operable to receive one or more inputs, the one or more inputs optionally including one or more of one or more female germ line genomes, one or more male germ line genomes, one or more probes and/or one or more molecule markers. In some embodiments, one or more sourcing units 420 are operable to provide one or more outputs, the one or more outputs optionally including one or more of one or more female germ line genomes, and/or one or more male germ line genomes. In some embodiments, the one or more male germ line genomes and/or the female germ line genomes are one or more haploid germ line genomes. In some embodiments, one or more male germ line genomes are one or more spermatid genomes, optionally one or more related spermatid genomes. In some embodiments, one or more female germ line genomes are one or more polar body genomes, optionally one or more related polar body genomes, and/or optionally one or more of one or more first polar body genomes or one or more second polar body genomes.

In some embodiments, one or more first locations are the same as one or more second locations, and/or one or more third locations, and optionally are included in one or more hybridization units 422, one or more monitoring units 424, one or more controller units 426, one or more computing units 428, one or more sequencing units, 430, one or more amplifying units 432, and/or one or more decondensing units 434. In some embodiments, one or more third locations, one or more second locations and/or one or more first locations are the same location.

In some embodiments, the one or more male germ line haploid genomes are at least partially isolated from one or more spermatozoa, and/or are part of one or more spermatozoa. In some embodiments, the one or more male germ line haploid genomes are at least partially isolated from one or more spermatids and/or are part of one or more spermatids. In some embodiments, the one or more male germ line haploid genomes are at least partially isolated from one or more spermatocytes and/or are part of one or more spermatocytes. In some embodiments, the one or more male germ line haploid genomes are at least partially condensed and/or are condensed. In some embodiments, the one or more male germ line haploid genomes are from one or more of animals, mammals, reptiles, birds or plants.

In some embodiments, one or more related spermatid genomes are part of one or more related spermatids and/or are at least partially isolated from one or more related spermatids. In some embodiments, one or more related spermatid genomes are from one or more of animals, mammals, reptiles, birds or plants.

In some embodiments, the one or more female germ line genomes are at least partially isolated from one or more of one or more ova, one or more oogonia, or one or more oocytes and/or are part of one or more of one or more ova, one or more oogonia, or one or more oocytes. In some embodiments, one or more female germ line genomes are from one or more of animals, mammals, reptiles, birds or plants.

In some embodiments, one or more related polar body genomes are part of one or more related polar bodies and/or are at least partially isolated from one or more related polar bodies. In some embodiments, one or more related polar body genomes are from one or more of animals, mammals, reptiles, birds or plants.

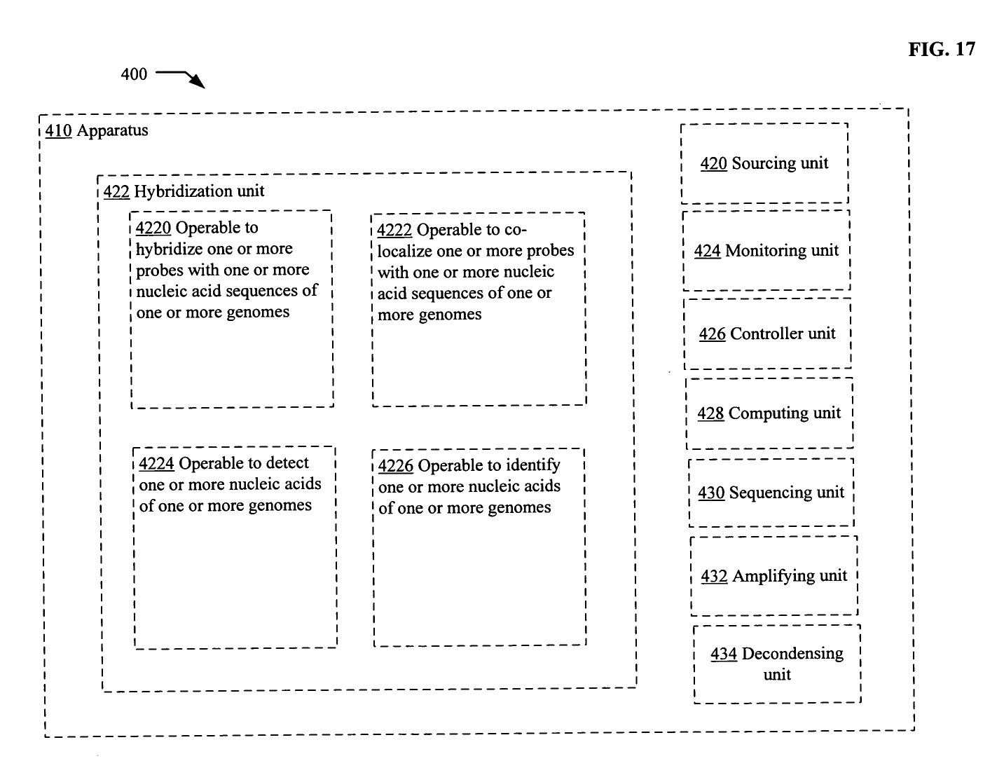

FIG. 17 shows a schematic 400 of illustrative embodiments of the optional apparatus 410 of FIG. 15, with specific illustrative embodiments of one or more hybridization units 422, including but not limited to, unit 4220, unit 4222, unit 4224, and/or unit 4226. In some embodiments, one or more hybridization units 422 are internal to the apparatus 410; in some embodiments, one or more hybridization units 422 are external to the apparatus 410. In some embodiments, one or more hybridization units 422 are part of the apparatus 410; in some embodiments, one or more hybridization units 422 are separate from the apparatus 410. In some embodiments, one or more hybridization units 422 are part of, the same as, and/or included in one or more of one or more characterization units 419, one or more sourcing units 420, one or more monitoring units 424, one or more controller units 426, one or more computing units 428, one or more sequencing units 430, one or more amplifying units 432, and/or one or more decondensing units 434.

In some embodiments, one or more hybridization units 422 are operable to detect one or more probes hybridized to one or more nucleic acids optionally of one or more male germ line haploid genomes and/or of one or more female germ line genomes. In some embodiments, one or more of the one or more hybridization units are operable to identify one or more of the one or more probes hybridized to one or more nucleic acids optionally of one or more male germ line haploid genomes and/or one or more female germ line genomes.

In some embodiments, one or more hybridization 422 units are operable to hybridize one or more probes with one or more nucleic acid sequences 4220 optionally of the one or more male germ line haploid genomes and/or one or more female germ line genomes. In some embodiments, one or more hybridization units are operable to co-localize one or more probes with one or more nucleic acid sequences 4222 of one or more male germ line haploid genomes and/or one or more female germ line genomes.

In some embodiments, one or more hybridization units 422 are operable to detect 4224, optionally destructively, one or more nucleic acid sequences of one or more genomes. In some embodiments, one or more hybridization units are operable to identify 4226, optionally destructively, one or more nucleic acid sequences of one or more genomes. In some embodiments, one or more hybridization units 422 are operable to detect 4224, optionally destructively, one or more probes hybridized to one or more nucleic acids optionally of one or more related spermatid genomes and/or of one or more related polar body genomes. In some embodiments, one or more hybridization units are operable to identify 4226, optionally destructively, one or more probes hybridized to one or more nucleic acids optionally of one or more related spermatid genomes and/or one or more related polar body genomes.

In some embodiments, one or more hybridization units 422 are operable to hybridize 4220, optionally destructively, one or more probes with one or more nucleic acid sequences optionally of the one or more related spermatid genomes and/or one or more related polar body genomes. In some embodiments, one or more hybridization units are operable to co-localize 4222, optionally destructively, one or more probes with one or more nucleic acids of one or more related spermatid genomes and/or one or more polar body genomes.

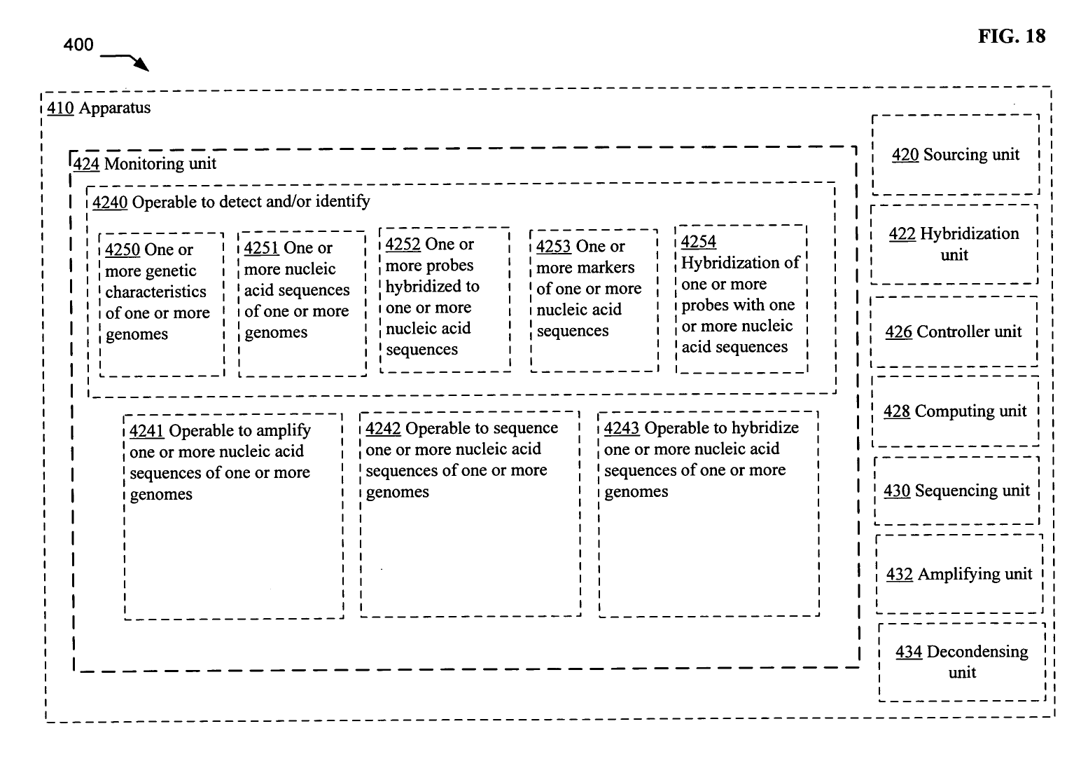

FIG. 18 shows a schematic 400 of illustrative embodiments of the optional apparatus 410 of FIG. 15, with specific illustrative embodiments of one or more monitoring units 424, including but not limited to, unit 4240, unit 4241, unit 4242, and/or unit 4243. In some embodiments, one or more monitoring units 424 are internal to the apparatus 410; in some embodiments, one or more monitoring units 424 are external to the apparatus 410. In some embodiments, one or more monitoring units 424 are part of the apparatus 410; in some embodiments, one or more monitoring units 424 are separate from the apparatus 410. In some embodiments, one or more monitoring units 424 are part of, the same as, and/or included in one or more of one or more characterization units 419, one or more sourcing units 420, one or more hybridization units 422, one or more controller units 426, one or more computing units 428, one or more sequencing units 430, one or more amplifying units 432, and/or one or more decondensing units 434.

In some embodiments, one or more monitoring units 424 are operable to detect and/or identify one or more genetic characteristics 4250 of one or more female germ line genomes and/or one or more male germ line haploid genomes. In some embodiments, one or more monitoring units are operable to detect and/or identify one or more nucleic acid sequences 4251 of one or more male germ line haploid genomes and/or one or more female germ line genomes.

In some embodiments, one or more monitoring units 424 are operable to detect association with, binding, and/or hybridization of one or more probes 4254 and/or one o more molecular markers with one or more nucleic acids of one or more male germ line haploid genomes and/or one or more female germ line genomes. In some embodiments, one or more monitoring units 424 are operable to detect and/or identify one or more probes or one or more molecular markers associated with, bound, and/or hybridized to one or more nucleic acids 4252 of one or more male germ line haploid genomes and/or one or more female germ line genomes.

In some embodiments, one or more monitoring units are operable to detect and/or identify, optionally destructively, optionally in situ, one or more genetic characteristics 4250 and/or one or more nucleic acid sequences 4251 of one or more related spermatid genomes and/or one or more related polar body genomes. In some embodiments, one or more monitoring units are operable to detect and/or identify, optionally destructively, optionally in situ, one or more markers of one or more nucleic acid sequences 4253 of one or more related spermatid genomes and/or one or more related polar body genomes. In some embodiments, one or more monitoring units are operable to detect and/or identify, optionally destructively, optionally in situ, one or more probes hybridized to one or more nucleic acid sequences 4252 of one or more related spermatid genomes and/or one or more related polar body genomes.

In some embodiments, one or more monitoring units are operable to amplify 4241, optionally destructively, optionally in situ, one or more nucleic acid sequences of one or more genomes. In some embodiments, one or more monitoring units are operable to sequence 4242, optionally destructively, optionally in situ, one or more nucleic acid sequences of one or more genomes. In some embodiments, one or more monitoring units are operable to hybridize 4243, optionally destructively, optionally in situ, one or more probes to one or more nucleic acid sequences of one or more genomes.

In some embodiments, one or more monitoring units are operable to amplify 4241, optionally destructively, optionally in situ, one or more nucleic acid sequences of one or more related spermatid genomes and/or one or more related polar body genomes. In some embodiments, one or more monitoring units are operable to sequence 4242, optionally destructively, optionally in situ, one or more nucleic acid sequences of one or more of the one or more related spermatid genomes and/or one or more related polar body genomes. In some embodiments, one or more monitoring units are operable to hybridize 4243, optionally destructively, optionally in situ, one or more probes to one or more nucleic acid sequences of the one or more related spermatid genomes and/or one or more related polar body genomes.

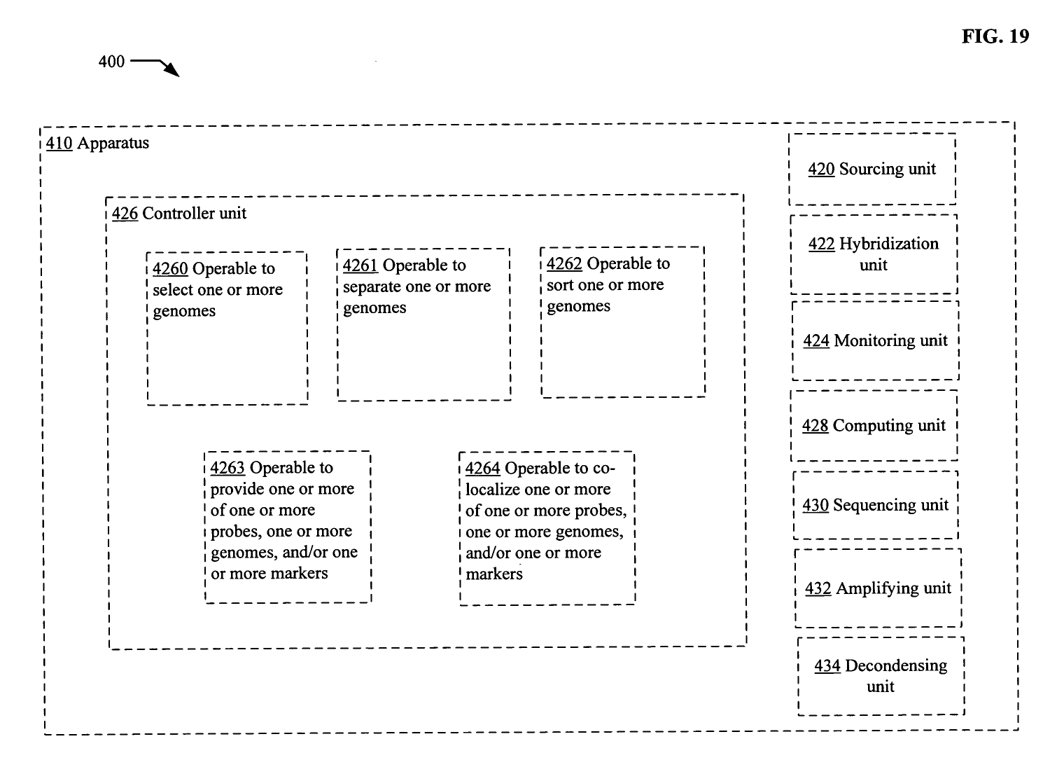

FIG. 19 shows a schematic 400 of illustrative embodiments of the optional apparatus 410 of FIG. 15, with specific illustrative embodiments of one or more controller units 426, including but not limited to, unit 4260, unit 4261, unit 4262, unit 4263 and/or unit 4264. In some embodiments, one or more controller units 426 are internal to the apparatus 410; in some embodiments, one or more controller units 426 are external to the apparatus 410. In some embodiments, one or more controller units 426 are part of, the same as, and/or included in one or more of one or more characterization units 419, one or more sourcing units 420, one or more hybridization units 422, one or more monitoring units 424, one or more computing units 428, one or more sequencing units 430, one or more amplifying units 432, and/or one or more decondensing units 434.

In some embodiments, one or more controller units 426 are operable to select 4260, separate 4261, and/or sort 4262 one or more of the one or more male germ line haploid genomes at least partially based on the one or more genetic characteristics of the one or more male germ line haploid genomes and/or a weighted combination of one or more genetic characteristics of one or more male germ line haploid genomes. In some embodiments, one or more controller units are operable to select 4260, separate 4261, and/or sort 4262 one or more of the one or more male germ line haploid genomes at least partially based on one or more genetic characteristics of one or more female germ line genomes and/or a weighted combination of one or more genetic characteristics of one or more female germ line genomes. In some embodiments, one or more controller units are operable to select 4260, separate 4261, and/or sort 4262 one or more of the one or more male germ line haploid genomes at least partially based on one or more of one or more target genetic characteristics or one or more reference genetic characteristics and/or a weighted combination of one or more of one or more target genetic characteristics or one or more reference genetic characteristics.

In some embodiments, one or more of the one or more controller units 426 are operable to select 4260, separate 4261, and/or sort 4262 one or more of the one or more male germ line haploid genomes optionally at least partially based on the detection and/or identification of one or more probes and/or molecular markers associated with, bound, and/or hybridized to one or more nucleic acids optionally of one or more male germ line haploid genomes and/or one or more female germ line genomes.

In some embodiments, one or more controller units 426 are operable to provide 4263 one or more probes to the one or more male germ line haploid genomes, and/or to provide 4263 one or more male germ line haploid genomes to one or more of the one or more probes. In some embodiments, one or more controller units are operable to provide 4263 one or more male germ line haploid genomes and/or one or more of the one or more probes to one or more first locations and/or to one or more hybridization units.

In some embodiments, one or more controller units 426 are operable to co-localize 4264 one or more probes with the one or more male germ line haploid genomes, and/or to co-localize 4264 one or more male germ line haploid genomes with one or more of the one or more probes. In some embodiments, one or more controller units are operable to co-localize 4264 one or more male germ line haploid genomes and/or one or more of the one or more probes at one or more first locations and/or at one or more hybridization units.

In some embodiments, the one or more male germ line haploid genomes are one or more related spermatid genomes. In some embodiments, the one or more female germ line genomes are one or more related polar body genomes, optionally one or more first polar body genomes and/or one or more second polar body genomes.

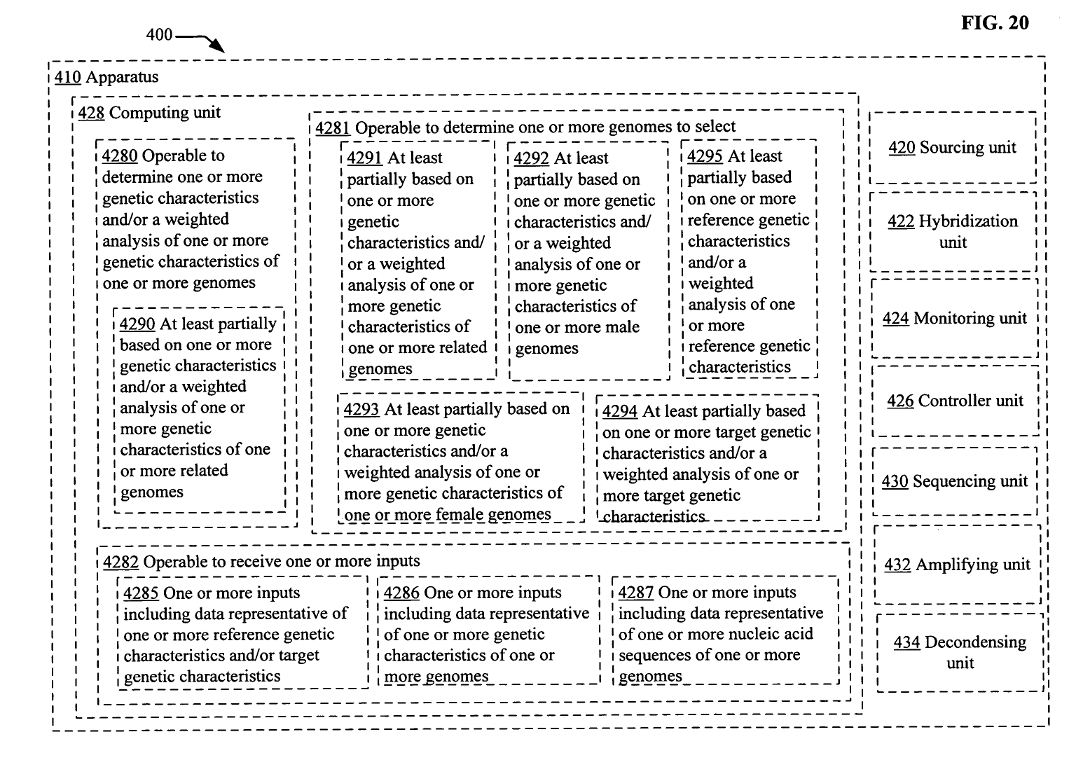

FIG. 20 shows a schematic 400 of illustrative embodiments of the optional apparatus 410 of FIG. 15, with specific illustrative embodiments of one or more computing units 428, including but not limited to, unit 4280, unit 4281, and/or unit 4282. In some embodiments, one or more computing units 428 are internal to the apparatus 410; in some embodiments, one or more computing units 428 are external to the apparatus 410. In some embodiments, one or more computing units 428 are part of, the same as, and/or included in one or more of one or more characterization units 419, one or more sourcing units 420, one or more hybridization units 422, one or more monitoring units 424, one or more controller units 426, one or more sequencing units 430, one or more amplifying units 432, and/or one or more decondensing units 434.

In some embodiments, one or more apparatus 410 further includes one or more computing units 428 operable to determine one or more genetic characteristics 4280 of one or more genomes and/or a weighted analysis of one or more genetic characteristics of one or more genomes.

In some embodiments, one or more computing units 428 are operable to determine one or more genetic characteristics and/or a weighted analysis of one or more genetic characteristics 4280 of one or more genomes, optionally one or more male germ line haploid genomes and/or one or more female germ line genomes, optionally at least partially based on detection and/or identification of one or more of the one or more probes hybridized to the one or more nucleic acids of one or more of the one or more male germ line haploid genomes. In some embodiments, one or more computing units are operable to determine one or more genetic characteristics and/or a weighted analysis of one or more genetic characteristics 4280 of one or more genomes, optionally one or more male germ line haploid genomes and/or one or more female germ line genomes, optionally at least partially based on the detected one or more genetic characteristics of the one or more male germ line haploid genomes.

In some embodiments, one or more computing units 428 are operable to determine one or more genetic characteristics and/or a weighted analysis of one or more genetic characteristics 4280 of one or more genomes, optionally one or more related spermatid genomes and/or one or more related polar body genomes, optionally at least partially based on detection and/or identification of one or more of the one or more probes and/or one or more molecular markers associated with, bound, and/or hybridized to the one or more nucleic acids of one or more related genomes. In some embodiments, one or more computing units are operable to determine one or more genetic characteristics and/or a weighted analysis of one or more genetic characteristics 4280 of one or more genomes, optionally one or more related spermatid genomes and/or one or more related polar body genomes, optionally at least partially based on the detected one or more genetic characteristics of the one or more related genomes.

In some embodiments, the one or more computing units 428 are operable to determine one or more genomes to select, sort, and/or separate 4281 at least partially based on one or more genetic characteristics of one or more related genomes, and/or based on a weighted analysis of one or more genetic characteristics of one or more related genomes 4291. In some embodiments, one or more related genomes are one or more related spermatid genomes and/or one or more related polar body genomes.

In some embodiments, the one or more computing units 428 are operable to determine one or more genomes, optionally one or more male germ line haploid genomes and/or one or more female germ line genomes, to select, sort, and/or separate 4281 at least partially based on one or more genetic characteristics of one or more male genomes, optionally one or more male germ line haploid genomes, and/or on a weighted analysis of one or more genetic characteristics of one or more male genomes, optionally one or more male germ line haploid genomes 4292.

In some embodiments, one or more computing units are operable to determine one or more genomes, optionally one or more male germ line haploid genomes and/or one or more female germ line genomes, to select, sort, and/or separate 4281 at least partially based on one or more genetic characteristics of one or more female genomes, optionally one or more female germ line genomes, and/or a weighted analysis of one or more genetic characteristics of one or more female genomes, optionally one or more female germ line genomes 4293.

In some embodiments, one or more computing units 428 are operable to determine one or more male genomes, optionally one or more male germ line haploid genomes and/or one or more female germ line genomes, to select, sort, and/or separate 4281 at least partially based on one or more of one or more target genetic characteristics or one or more reference genetic characteristics and/or a weighted combination of one or more of one or more target genetic characteristics 4294 or one or more reference genetic characteristics 4295.

In some embodiments, one or more computing units 428 are operable to receive one or more inputs 4282, the one or more inputs optionally including data representative of one or more genetic characteristics 4286 and/or one or more nucleic acid sequences 4287 of one or more genomes, optionally one or more female germ line genomes and/or one or more male germ line genomes. In some embodiments, the one or more female germ line genomes and/or the one or more male germ line genomes are one or more haploid genomes. In some embodiments, one or more computing units 428 are operable to receive one or more inputs 4282, the one or more inputs optionally including data representative of one or more of one or more target genetic characteristics and/or one or more reference genetic characteristics 4285.

In some embodiments, the one or more male germ line haploid genomes are one or more related spermatid genomes 4291. In some embodiments, the one or more female germ line genomes are one or more related genomes 4291, optionally one or more polar body genomes, optionally one or more first polar body genomes and/or one or more second polar body genomes.

Materials and reagents described in the Examples are commercially available, unless otherwise specified.

### EXAMPLE 1

### Mammalian Spermatozoa Selection Based on Nucleic Acid Hybridization with Peptide Nucleic Acid

Sperm cells from, for example, boar, bull, stallion or ram, are collected using known animal husbandry methods including using a gloved-hand, an artificial vagina, and/or electro-ejaculation methods as appropriate.

After collection, the semen is diluted with a species-specific buffer to extend the lifespan of the sperm outside the body (e.g. artificial insemination buffer). Appropriate diluents provide energy and nutrients, buffering action for pH changes (e.g. due to lactic acid formation), protection from temperature shock (e.g. rapid cooling), maintain osmotic pressure, balance electrolytes, inhibit microorganism growth, as well as facilitating dilution to an appropriate volume for hybridization and selection. For example, a 2.9% sodium citrate—egg yolk buffer may be used for cattle (see, e.g., J. Dairy Sci. (1941) 24:905), and a Beltsville Thaw Solution (BTS) may be used for boar sperm.

The DNA sequence of interest is identified. Such DNA sequence can be, for example, a trait locus, a particular allele, or other DNA sequence targeted for hybridization.

Based on the sequence of the DNA to be targeted, peptide nucleic acids that bind the target DNA sequence are designed and constructed following the procedures described, for example, by Eur. J. Hum. Genetics (2003) 11:337-341; Mammalian Genome (2000) 11:384-391; Adv. in Genetics (2006) 56:1-51; EMBO J (2003) 22:6631-6641; Mammalian Genome (1999) 10:13-18; or Mol. Hum. Repro. (2004) 10:467-472). Alternatively, the peptide nucleic acids may be synthesized using an Applied Biosystems 3400 DNA Synthesizer or an ABI 3900 Synthesizer, or using custom commercial services.

Peptide nucleic acids (from, for example, Applied Biosystems) may be conjugated with various fluorescent dyes such as FITC, TRITC, and/or BODIPY® derivatives, for example, and/or quantum dots (see, e.g., Histochem. Cell Biol. (2006) 125:451-456). BODIPY® dyes are membrane soluble, aiding penetration of probes (from, for example, Molecular Probes Inc.; described in, for example, U.S. Pat. No. 5,338,854 or U.S. Pat. No. 4,774,339). PNA probes are used at a final concentration of about 0.1 to about 100 μM depending on the cell concentration, among other things.

Labeled or unlabeled peptide nucleic acids are added to diluted sperm samples under conditions to effect hybridization while minimizing the impact on motility and/or viability. In some instances, peptide nucleic acid probes with fluorescent tags may readily penetrate the cells, travel to the nucleus, and bind nuclear DNA. Optionally, cell penetration is facilitated by methods known in the art including electroporating, chemically shocking (e.g. using glycerol and/or DMSO), liposome-encapsulating, micro-injecting, DEAE-dextran-mediated transferring, co-precipitating with calcium phosphate, and/or adding cell-permeation enhancing solutions such as mild surfactants and/or DMSO.

Hybridization incubations may range from about 30 minutes to about 24 hours or about 144 hours or longer, depending on the ease of uptake into the cell nucleus and target binding. Hybridization temperatures may range from the thermotropic phase transition temperature of the membranes of the sperm, to room temperature (approximately 23° C.), to less than about 30° C., or to less than about 39° C.

Cells with fluorescently labeled peptide nucleic acids that hybridized to target nucleic acids are identified by detection of their emitted fluorescence using conventional methods. Following hybridization, cells are sorted, using for example flow cytometry or microfluorometry, based on differences in quantitative and/or qualitative fluorescence to produce subpopulations enriched or depleted in cells with one or more target sequences. Cells may also be sorted using fluorescent microscopy. Methods for effecting flow cytometry separations while minimizing the impact on cell motility and/or viability are known in the art (e.g. U.S. Pat. No. 5,135,759, or U.S. Pat. No. 5,985,216), and appropriate systems have been described herein, and in WO 03/020877, for example.

### EXAMPLE 2

### Mammalian Spermatozoa Nuclei Selection Based on Nucleic Acid Hybridization with Peptide Nucleic Acid

Methods of isolating sperm nuclei are known in the art (see, e.g., Hum. Reprod. (2005) 20:2784-2789). Semen is washed three times by centrifugation at 1620 g for 10 minutes in, for example, 50 mmol/L Tris-HCl, pH 7.2 and 0.15 mol/L NaCl (10× sample volume). Sperm pellets are resuspended in, for example, 2.6 ml of the same buffer containing 1% SDS, incubated for 15 minutes at room temperature, and sonicated six times for 15 seconds each at 200 W using, for example, a Branson sonifier cell disrupter, model W 140 (Branson Sonic Power Co., Plainview, N.Y.). Sonicated cell solutions are centrifuged at 3500 g for 1 hour through a 1.1 mol/L sucrose in 50 mmol/L Tris-HCl, pH 7.2 gradient. Pellets are washed twice by centrifugation at 1620 g for 10 minutes in, for example, 50 mmol/L Tris-HCl, pH 7.2. Lack of contamination of the nuclear fraction may be assessed by microscopic examination, for example.

PNAs are designed and constructed using methods and materials described herein or known in the art. Hybridization of the PNA is induced using the methods and materials described herein or known in the art.

Following hybridization, nuclei are sorted, using for example flow cytometry or microfluorometry, based on differences in quantitative and/or qualitative fluorescence to produce subpopulations enriched or depleted in nuclei with one or more target sequences. Alternatively, nulcei may be sorted using fluorescent microscopy. Methods for effecting flow cytometry separations while minimizing the impact on nuclei viability are known in the art (e.g. U.S. Pat. No. 5,135,759 or U.S. Pat. No. 5,985,216), and appropriate systems have been described herein, and, for example, in WO 03/020877.

### EXAMPLE 3

### Decondensation and Nucleic Acid-Based Selection of Mammalian Spermatozoa

Semen samples are obtained using methods known in the art and/or described herein. Semen may be allowed to liquefy at room temperature for approximately 30 min to 3 hours.

Semen solutions are demembranated, for example by diluting 1:10 in a demembranating solution pre-warmed to approximately 37° C., or other appropriate temperature based, for example, on the body temperature of the species from which the sperm is recovered. Demembranating solutions are known in the art (see, e.g., J. Exp. Zoology (1999) 284:789-797), and can be modified to achieve the desired extent of demembranation by altering, for example, the concentration of Triton X-100 (specially purified for membrane research, available from, for example, Boehringer-Mannhein, Germany) in the solution from approximately 0.01%, 0.015%, 0.017%, 0.02%, to 0.022%, for example. During demembranation, samples may be stirred for 20 seconds, and allowed to sit unstirred for 25 seconds.

Demembranated semen samples are decondensed, for example by diluting 1:10 with decondensing solution pre-warmed to approximately 37° C. (or other appropriate temperature as discussed above), stirred briefly, and then allowed to incubate at 37° C. for approximately 30 seconds, 5 minutes, 10 minutes or 15 minutes depending on the species and extent of decondensation desired. Decondensation solutions are known in the art (see, e.g., J. Exp. Zoology (1999) 284:789-797), and may include 24 mM potassium glutamate, 192 mM sucrose, 1.2 mM MgSO₄, 19.2 mM Hepes (N-2-hydroxyethylpiperazine-N-2-ethanesulfonic acid), 5.8 mM EDTA (ethylene diamine tetraacetic acid), 2.9 mM DTT (dithiothreitol, Sigma D-0632), 48 μM cAMP (adenosine 3′:5′-cyclic monophosphate; Sigma A-6885), and 4.03 USP units/ml heparin (sodium salt grade 1: from porcine intestinal mucosa, Sigma), pH 7.8.

Demembranated and/or decondensed sperm samples may be diluted 1:10 in reactivating solution pre-warmed to approximately 37° C. (or other appropriate temperature as discussed above), stirred briefly, and then allowed to incubate at 37° C. for a few minutes. Reactivating solutions are known in the art (e.g. J. Exp. Zoology (1999) 284:789-797), and may include 5 mM adenosine 5′-triphosphate (ATP, Sigma A-5394) and 2.5 mM MgSO₄.

Sperm demembranation, decondensation, and/or reactivation can be monitored using microscopic examination, for example.

Decondensed, and optionally reactivated, sperm may be exposed to one or more nucleic acid binding solutions including polyamide, peptide nucleic acid, and/or oligonucleic acid probes under conditions to facilitate binding and/or hybridization.

### EXAMPLE 4

### Decondensation and Nucleic Acid-Based Selection of Mammalian Spermatozoa and Isolated Nuclei

Semen may be obtained by methods described herein and/or in the art. Semen samples are allowed to liquefy up to one hour prior to capacitation or nuclei isolation.

Methods for capacitating semen samples are known in the art (see, e.g., Hum. Reprod. (2005) 20:2784-2789). Semen samples are washed twice by centrifugation at 300 g for 10 minutes in a 1:5 dilution of tubal fluid medium (e.g. HTF; from, for example, Irvine Scientific, Santa Ana, Calif.) supplemented with 0.3% bovine serum albumin (BSA). The sperm pellet is overlaid with 1 ml fresh HTF with 2.6% BSA (HTF-26B) for 90 minutes at about 37° C. (or other appropriate temperature for the species) in an atmosphere of 5% CO₂ in air.

Sperm nuclei then are isolated using methods known in the art, or as described herein, for example in Example 2 above.

Methods of decondensing capacitated sperm and isolated nuclei are known in the art (see, e.g., Hum. Reprod. (2005) 20:2784-2789). Sperm and isolated nuclei are incubated in HTF with 46 μmol/L Heparin and 10 mmol/L glutathione (GSH) for approximately 15, 30 or 60 minutes at 37° C. (or other appropriate temperature for the species) in an atmosphere of 5% CO₂ in air. The extent of decondensation can be assessed by phase-contrast in an Olympus CH2 microscope at 400× magnification, for example.

Decondensed sperm and/or isolated nuclei can be exposed to one or more nucleic acid binding solutions including, for example, polyamide, peptide nucleic acid, and/or oligonucleic acid probes under conditions to facilitate binding and/or hybridization, as appropriate.

### EXAMPLE 5

### Decondensation of Mammalian Spermatozoa

Methods for decondensing mammalian sperm are known in the art (see, e.g., Theriogenology (2005) 63:783-794). Frozen or fresh sperm (from e.g. boar, bovine) may be incubated in Dulbecco's phosphate buffered saline (DPBS; Life Technologies) supplemented with 0.1% polyvinyl alcohol (PVA) and 5 mM DTT for approximately 50 minutes. Other appropriate buffers may be used, and the final DTT concentration varied depending on the species of the sperm. Sperm are washed three times before fertilization by centrifugation at 400 g for 5 minutes in 2 ml DPBS-PVA without 5 mM DTT.

### EXAMPLE 6

### Recondensation of Selected Mammalian Spermatozoa and Isolated Nuclei

Methods for recondensing DNA are known in the art and include incubation with protamine in a low ionic strength buffer (see, e.g., J. Biol. Chem. (2004) 279:20088-20095). Partially and/or completely decondensed sperm and/or isolated nuclei can be partially and/or completely recondensed by incubation with protamine in a low ionic strength buffer.

Protamine can be isolated from sperm cells (e.g. bull protamine from bull sperm cells, or species specific to the sperm and/or isolated nuclei) by methods known in the art (see, e.g., J. Biol. Chem. (2004) 279:20088-20095). Isolated sperm cell chromatin is solubilized in 2.6 M urea, 1.1 M NaCl, 0.9 M guanidine hydrochloride (GuCl), and 150 mM 2-mercaptoethanol, and DNA is precipitated from the solution with concentrated HCl. The protamine solution is dialyzed against 10 mM HCl, and the protamine is precipitated with trichloroacetic acid, washed in acetone, and dissolved in dH₂O.

Solubilized protamine is filter-sterilized using, for example, Amicon Ultrafree-MC centrifugal filters with 0.22 μm pore diameter (Millipore). Partially or completely decondensed sperm and/or isolated nuclei are incubated in a solution including approximately 2.25 μM protamine, 10 mM sodium cacodylate, and 100 μM EDTA (pH 7.5) for approximately 10 minutes, 30 minutes, one hour, two hours, to at least three hours at 37° C. (or other appropriate temperature for the species) in an atmosphere of 5% CO₂ in air.

### EXAMPLE 7

### Mammalian Female Reproductive Cell and/or Isolated Nuclei Selection Based on Nucleic Acid Hybridization and/or Binding

Female reproductive cells, including oocytes, ova, and/or polar bodies, from, for example, cows, sows, ewes, and mares are collected using known animal husbandry methods including, for example, super-ovulation, in vitro production, and collection at slaughter. Mice, for example, may be superovulated by consecutive injections of eCG (5IU) and hCG (5 IU) 48 hours apart. About 14 hours following hCG injections, oocyte-cumulus complexes are released from oviducts into Hepes-CZB. Cumulus cells can be dispersed by 5 minutes treatment with 0.1% bovine testicular hyaluronidase (300 USP units/mg; from, for example, ICN Pharmaceuticals, Costa Mesa, Calif.) in Hepes-CZB (see, e.g., Biol. Reprod. (1998) 59:100-104).

After collection, female reproductive cells are cultured and/or maintained in a variety of balanced salt solutions (e.g. TC 199, M16, NCSU23) known in the art at appropriate temperatures, for example ones resembling the body temperature the species from which the cell was isolated (e.g. mice at 37° C., pig at 39° C.). Cumulus-free mice oocytes can be kept in CZB at 37.5° C. under 5% CO2 in air. Appropriate solutions and temperatures extend the length of cell viability and function and may be modified as appropriate (see, e.g., J. Cell. Biol. (1986) 102:568).

Methods of designing and constructing probes to bind and/or hybridize to target DNA sequences such as those indicative of a particular allele, trait locus, or other feature of interest are known in the art and described herein. Probes may include peptide nucleic acids, polyamides, and/or oligonucleotides, among others, and may be tagged with one or more tags known in the art and/or described herein.

Methods of imaging molecules in living cells and isolated cell nuclei are known in the art and described herein (see, e.g., Histochem. Cell Biol. (2006) 125:451-456; Biochem. Biophys. Res. Commun. (2006) 344:772-779; or Nature (2004) 5:856-862). One approach is to use quenched probes that fluoresce only when hybridized/bound to the target nucleic acid sequence (see, e.g., Trends in Biotech. (2005) 23:225-230; or Curr. Organic Chem. (2006) 10:491-518).

Probes are provided to the nucleus, using for example, mild membrane permeabilization, microinjection, and/or probes amenable to uptake, such as polyamides and peptide nucleic acids.

Following hybridization, cells and/or nuclei are sorted, using for example flow cytometry or microfluorometry, based on differences in quantitative and/or qualitative fluorescence to produce subpopulations enriched or depleted in cells and/or nuclei with one or more target sequences. Alternatively, cells and/or nuclei can be sorted using fluorescent microscopy. Methods for effecting flow cytometry separations while minimizing the impact on cells and/or nuclei viability are known in the art (see, e.g., U.S. Pat. No. 5,135,759, or U.S. Pat. No. 5,985,216), and appropriate systems have been described herein, and in WO 03/020877, for example.

### EXAMPLE 8

### Fertilization Using Polar Body Genomes

Female reproductive cells, including oocytes, ova, and/or polar bodies, from, for example, cows, sows, ewes, and mares are collected, cultured and maintained using methods described above in Example 7 or known in the art.

Methods for enucleating recipient oocytes are known in the art (see, e.g., Biol. Reprod. (1998) 59:100-104). Enucleation of mice mature oocytes is performed using, for example, Hepes-CZB containing 5 μg/ml cytocholasin B (25° C. for 10 minutes). Oocytes held by a pipette are rotated until detection of a small, translucent ooplasmic spot—the location of metaphase II chromosomes. The zona pellucida is drilled with an enucleation pipette (approx. 10 μm inner diameter) by applying a few piezo pulses, and its tip is advanced until it reaches the translucent spot identified above. The translucent spot (and metaphase chromosomes) are drawn into the pipette gently, without breaking the plasma membrane, and pulled away from the oocyte until a stretched cytoplasmic bridge breaks off. Success of enucleation may be assessed using Hoechst 33342 staining.

Methods for transferring first polar bodies into enucleated oocytes are known in the art (see, e.g., Biol. Reprod. (1998) 59:100-104). The zona pellucida of oocytes with live polar bodies (assessed according to, for example, Live/Dead FertiLight; Molecular Probes, Inc. Eugene Oreg.) are drilled into with a piezo-driven injection pipette. The plasma membrane of the polar body may be broken when sucking into the pipette. The entire contents are injected into an enucleated oocyte, and are incubated in CZB for 2 hours at 37.5° C. under CO₂ in air prior to fertilization.

Methods for transferring second polar bodies into enucleated oocytes and/or nucleated zygotes are known in the art (see, e.g., J. Reprod. Fertility (1997) 110:263-266). Second polar bodies and female pronuclei may be removed from zygotes through the zonae pellucidae using micromanipulators under an inverted microscope with Nomarski optics, for example. The second polar body is inserted into the perivitelline space of a recipient zygote with one pronucleus, and placed in a drop (10 μl) of fusion medium (300 mmol/L mannitol, 0.05 mmol/L CaCl₂, 0.1 mmol/L MgSO₄, 5 mg/ml polyvinylpyrrolidone) between the electrodes of a circular electrofusion chamber (from, for example, Shimadzu, Kyoto). The width and depth of the electrode gap are 0.5 and 2.0 mm, respectively, and electrofusion is induced by applying 20 V/cm AC for 30 seconds, 3000 V/cm DC for 10 μs, and 20 C/cm AC for 90 seconds, consecutively.

### EXAMPLE 9

### Selection of Ova Using First Polar Body and/or Second Polar Body Genetic Information

First and/or second polar bodies from oocytes are obtained using methods known in the art (e.g. Biol. Reprod. (1998) 59:100-104; J. Reprod. Fertility (1997) 110:263-266; Reproductive BioMedicine Online (2003) 6:403-409; Mol. Hum. Reprod. (1999) 5:89-95). Oviductal oocytes may be collected from mice, for example, between 13 and 17 hours after hCG injection, and the viability of the first polar body assessed (as above, for example). The second polar body may be extruded following parthenogenic activation or fertilization.

Genetic analysis of the polar bodies is performed using methods known in the art (see, e.g., Mol. Hum. Reprod. (1999) 5:89-95; Fertility and Sterility (2002) 78:543-549; J. Assisted Reprod & Genetics (1998) 15: 253-257; Prenat. Diagn. (2000) 20:1067-1071; Reprod. BioMed. OnLine (2002) 4:183-196; Prenat. Giagn. (2002) 21:767-780; or Mol. Cell. Endocrinol. (2001) 183:S47-S49). Based on the information gathered from genetic analysis of one or more of the polar bodies, and compared with the genetic information of diploid cells, the genetic information of the haploid ova can be determined. The desired ovum can be selected and electrofused, for example, with a selected male pronuclei.

### EXAMPLE 10

### Selection of Spermatids and/or at Least Partially Isolated Spermatid Nuclei

Spermatids from mature male mammals are isolated using methods known in the art (see, e.g., Development (1995) 121:2397-2405). Seminiferous tubules isolated from a mature male mouse testis are placed in 1 ml of cold (4-10° C.) 0.9% NaCl containing 1% (W/v) polyvinyl pyrrolidone (PVP, Mr 360×10³, ICN), and are cut into minute pieces. The seminiferous tubule suspension is mixed thoroughly with repeated pipettings with 2 ml cold PVP-saline (0.9% NaCl, 12% (w/v) PVP) to release spermatozoa, spermatids, and other reproductive cells. Cells can be identified in droplets on a Petrie dish, for example, covered with mineral oil. Cells can be maintained at approximately 16-17° C. for several hours (e.g. three hours) during this process. Round spermatids can be recognized by their small size and centrally located chromatin mass.

Spermatid clones that are connected by stable cytoplasmic bridges (or ring canals) are identified and isolated using methods known in the art (see, e.g., Mol. Biol. Cell (2003) 14:2768-2780; or Histochem. Cell Biol. (1997) 108:77-81). Seminiferous vesicles are dissected free from the interstitial tissue in a Petrie dish containing phosphate-buffered saline solution, pH 7.4. The transillumination pattern may be identified under stereomicroscope, for example, and tubules at stages I-IV of the cycle are selected and cut into approximately 0.5 mm to 1 mm segments. The cells within the tubules can be extruded by lowering a cover slip (20×20 mm), for example, over the tubule allowing wicking of excess fluid to create a slightly flattened monolayer (under 40× phase-contrast optics). The spermatid clones are separated, and binding and/or hybridization procedures are performed on one, two, three, and/or four of the spermatids of a given clone using methods described herein and/or known in the art.

The hybridization/binding patterns of the probes to the nucleic acid sequences of the individual spermatid clones are compared with the known sequence or binding/hybridization pattern for diploid cells of the donor organism. Through a process of comparison and elimination, the predicted identity of the nucleic acid sequences in a spermatid clone can be determined, and the desired spermatid selected.

### EXAMPLE 11

### Fertilization Using Spermatids and/or at Least Partially Isolated Spermatid Nuclei

Oocytes are fertilized with spermatids and/or isolated nuclei using methods that are known in the art (see, e.g., Development (1995) 121:2397-2405). Whole spermatids are sucked into an injection pipette (4-10 μm internal diameter) that is attached to a Piezo electric pipette driving unit (e.g. Model PMM-10, Prima Meat Packers, Tsuchiura, Japan); partially isolated nuclei can be obtained by drawing spermatids in and out of 4 μm internal diameter injection pipettes. The zona pellucida of a mature unfertilized oocyte is drilled and the oolemma is broken by applying Piezo pulses. The entire spermatid, with or without an intact plasma membrane, or the at least partially isolated nucleus, is expelled into the ooplasm, and the pipette tip is gently withdrawn.

### EXAMPLE 12

### Chromosome Selection

The DNA sequence or sequences for selection of a specific chromosome or chromosomes are identified. Such sequence can be, for example, a trait locus, a particular allele, or other DNA sequence targeted for selection. Information regarding mammalian genomic sequence, as well as trait and disease linkages, may be obtained from the literature and from public databases (see, e.g., bovine genome sequence, Snelling et al. (2007) Genome Biology 8:R165 published on line ahead of publication; pig genome sequence, Humphray et al. (2007) Genome Biology 8:R139 published on line ahead of publication; Online Mendelian Inheritance in Animals (OMIA) and Online Mendelian Inheritance in Man (OMIM) at the National Center for Biotechnology Information).

A trait locus, a particular allele, or other DNA sequence targeted for selection may be located on a single chromosome. As such, a single chromosome may be targeted for selection. Alternatively, a quantitative trait may be represented on multiple chromosomes. For example, the quantitative trait loci or multiple haplotypes associated with backfat thickness in beef cattle have been mapped to bovine chromosomes 2, 5, 6, 19, 21, and 23 (Li et al. (2004) J. Anim. Sci. 82:967-972). Similarly, growth traits and meat quality of specific breeds of pigs are associated with quantitative trait loci on chromosomes 1, 2 and 7 (Sanchez et al. (2006) J. Anim. Sci. 84:526-537). As a result, multiple chromosomes may be selected for a specific trait or phenotype. In some instances, multiple chromosomes associated with multiple traits or phenotypes may be selected to generate an optimized genome.

Chromosome selection may be destructive or non-destructive to the cell. The cell may be a germ line cell, such as, for example, a sperm, a spermatogonia, an oocyte, or a stem cell. Alternatively, the cell may be a somatic cell, including for example, a stem cell or a progenitor cell. For non-destructive selection, a chromosome or chromosomes may be screened in an intact cell for the presence or absence of a specific sequence, for example. The cell containing the selected chromosome or chromosomes may be immediately used for fertilization, for example. Alternatively, the cell containing the selected chromosome or chromosomes may be propagated, frozen, and stored for future use. Optionally, the selected chromosome or chromosomes from a cell are removed and placed into another cell.

Chromosome selection may also be destructive to the cell, but non-destructive to the chromosome or chromosomes. As a result, chromosomes may be isolated from a cell and screened using a non-destructive method. The selected chromosome or chromosomes may be placed directly into either a sperm or an oocyte for immediate fertilization. Alternatively, the selected chromosome or chromosomes may be placed into a cell for propagation and future use.

Alternatively, chromosome selection may be destructive to both the cell and the chromosome such that the chromosomes are lost in the process of screening. In this instance, the source of the chromosomes may be a somatic cell, for example, from an individual or individuals. After a specific chromosome or chromosomes has been selected from a particular somatic cell of a particular individual, additional somatic cells may be acquired from that individual and the specific chromosome or chromosomes removed. Alternatively, a male or female germ line cell may be used as a source of chromosomes for selection. As described above, the selected chromosome or chromosomes may be immediately used in a sperm or oocyte, or may be placed into cells for propagation and future use.

Chromosome selection may be carried out using, for example, a protein nucleic acid (PNA) probe that specifically binds the target DNA sequence on the chromosome of interest. Probes are designed and constructed using the procedures, for example, described by Pellestor et al. (2003) Eur. J. Hum. Genetics 11:337-341; Chen et al. (2000) Mamm. Genome 11:384-391; Lundin et al. (2006) Adv.Genet. 56:1-51; Molenaar et al. (2003) EMBO J. 22:6631-6641; Chen et al. (1999) Mamm. Genome 10:13-18; or Paulasova et al. (2004) Mol. Hum. Repro. 10:467-472). Alternatively, the PNA may be synthesized using an Applied Biosystems 3400 DNA Synthesizer or an ABI 3900 Synthesizer, or using custom commercial services (e.g. Panagene, Daejeon, Korea).

Peptide nucleic acids (from, for example, Panagene) may be conjugated with various dyes such as FAM, FITC, OregonGreen488, TAMRA, AlexaFluor488, TexasRed, AlexaFluor532, Cy3, Cy5, for example, and/or quantum dots (see e.g. Dahan (2006) Histochem. Cell. Biol. 125:451-456). PNA probes are used at a final concentration of about 0.1 to about 100 μM depending on the cell concentration, among other things.

Labeled PNA probes may be added to diluted cell samples under conditions to effect probe hybridization while minimizing the impact on cell viability. In some instances, PNA probes with fluorescent tags readily penetrate the cells, travel to the nucleus, and bind nuclear DNA. Optionally, cell penetration is facilitated by methods known in the art, including electroporating, chemically shocking (e.g. using glycerol and/or DMSO), liposome-encapsulating, microinjecting, DEAE-dextran-mediated transferring, co-precipitating with calcium phosphate, and/or adding cell-permeation enhancing solutions such as mild surfactants and/or DMSO. Alternatively, probes may be incorporated into cells using streptolysin O, scrape-loading, or peptide-mediated membrane transfer (Tanke et al. (2005) Curr. Opin. Biotechnol. 16:49-54).

Optionally, chromosome selection may be carried out on live cells using tagged sequence-specific polyamides (see, e.g. WO 03/020877 A2; Edelson et al. (2004) Nucleic Acids Res. 32:2802-2818; Crowley et al. (2003) Bioorg. Med. Chem. Lett. 13:1565-1570).

Chromosome selection may also be carried out using a variety of additional methods including restriction landmark genomic scanning (RLGS), southern blot analysis combined with restriction fragment length polymorphism (RFLP), fluorescence in situ hybridization (FISH), enzyme mismatch cleavage (EMC) of nucleic acid heteroduplexes, ligase chain reaction (LCR), and polymerase chain reaction (PCR) based methods (Tawata et al. (2000) Comb. Chem. High Throughput Screen. 3:1-9).

RLSG may be used to scan an entire mammalian genome. As such, genomic DNA is digested with one or two restriction enzymes with eight-base recognition sites to generate DNA fragments greater than 100 kb in size. The fragments are separated on an agarose gel, digested with one or more restriction enzymes within the agarose gel, and then separated in a second dimension by polyacrylamide gel electrophoresis (PAGE) (Tawata et al. (2000) Comb. Chem. High Throughput Screen. 3:1-9). The fragments may be stained nonspecifically with an intercalating dye, for example. The resulting pattern may be compared with pre-established norms, for example, to detect genetic mutations.

Southern analysis combined with RFLP may be used to select for a chromosome or chromosomes. In this instance, genomic DNA is digested with one of more restriction enzymes, separated on an agarose gel and transferred to a membrane for hybridization with a gene specific probe.

FISH may be used to detect deletions, duplications and/or translocations of genes on specific chromosomes in situ. In this instance, fluorescent complimentary DNA probes are hybridized to condensed metaphase, early prophase or interphase chromosomes from dividing cells prepared, for example, as a metaphase spread. For example, FISH may be used to detect Robertsonian translocations, a common chromosome rearrangement in mammals characterized by chromosome breaking at the centromere and fusion to form a morphologically distinct chromosome.

For example, a 1/29 translocation, in which a chromosome of chromosome pair 1 and a chromosome of chromosome pair 29 have fused, is a common translocation in bovine animals. It is associated with significant reductions in the fertility of cows bred by artificial insemination. Early embryonic death occurs in embryos produced by fertilization of affected gametes or by fertilization of normal gametes by spermatozoa carrying the 1/29 translocation. As such, bovine chromosomes, for example, may be screened by FISH for the 1/29 translocation using, for example, a commercially available kit such as the Star*FISH© Bovine Translocation (1/29) FISH Kit (Cambrio, Cambridge, UK).

Optionally, FISH may be used in combination with chromosome-specific paints that hybridize to all or a large portion of a given chromosome in a process called spectral karyotyping (see, e.g., Schrock et al. (1996) Science 273:494-497). Chromosome paints may be used to detect chromosomal translocations as well as deletions, inversions and amplifications (see, e.g., U.S. Pat. No. 6,270,971). Chromosome paints for a given chromosome in a mammalian genome may be generated by isolation of a specific chromosome, followed by labeling of the DNA with one of a variety of fluorochromes including FITC, the cyanines Cy2, Cy3, Cy3.5, Cy5, and Cy7, Texas Red, rhodamine, lissamine and phycoerythrin. Alternatively, chromosome paints may be derived from overlapping bacterial artificial chromosome (BAC) constructs, for example, carrying a mammalian genome or from overlapping PCR fragments generated from a specific chromosome (see, e.g. Thalhammer et al. (2004) Chromosome Res. 12:337-343). Alternatively, chromosome paints may be acquired from commercial sources (from, for example, Cambio, Cambridge, UK).

A variety of PCR related methods may be used to select for chromosomes with specific characteristics, and may be used for both known mutations and unknown mutations (Tawata et al. (2000) Comb. Chem. High Throughput Screen. 3:1-9). For known mutations, specific PCR oligonucleotide probes are designed to bind directly to the mutation or proximal to the mutation.

For example, PCR may be used in combination with RFLP. In this instance, a DNA fragment or fragments generated by PCR with primers on either side of the mutation site are treated with restriction enzymes and separated by agarose gel electrophoresis. The fragments themselves may be detected using an intercalating dye such as, for example, ethidium bromide. An aberrant banding pattern may be observed if mutations exist within the restriction sites. PAGE may be used to detect single base differences in the size of a fragment. This approach may be used, for example, to assess genetic variability in the equine ELA-DRB Class II Major Histocompatibility Complex, variation in which may play an essential role in recognizing and resisting parasites (Peral-Garcia et al. (1999) J. Equine Sci. 10:13-16; Hedrick et al. (1999) Genetics 152:1701-1710.

Alternatively, PCR may be used in combination with DNA sequencing to select for chromosomes with specific characteristics. For example, PCR combined with sequencing may be used to assess sequence variations associated with coat color, for example, in horses (Haase et al. (2007) PLOS Genetics, published on-line ahead of publication). The four depigmentation phenotypes, roan, sabino-1, tobiano and dominant white, are associated with mutations in the c-Kit protooncogene gene (c-Kit), which encodes for a tyrosine kinase. PCR primers may be designed to generate a fragment that spans a potential mutation site. For example, the depigmentation phenotype is associated with a single point mutation in c-Kit which leads to truncation of the encoded kinase. As such, PCR in combination with sequence analysis may be used to select for or against the depigmentation phenotype. This approach may be used to assess the sequence of other genes associated with coat color in a number of mammalian species, including, for example, tyrosinase-related protein 1 (Tyrp 1), tryosinase (Tyr), myosin 5a (Myo 5a), endothelin receptor B (Ednrb), and mast cell growth factor (Mgf) (see, e.g., Barsh (2001) Genetics Encyclopedia: Coat Color Mutations, Animals).

Similarly, PCR in combination with DNA sequencing may be used to select for or against increased muscle mass in a mammal. For example, a 2 base pair deletion in the whippet myostatin gene (MSTN) in the homozygous state results in a double-muscling phenotype commonly referred to as the “bully” whippet and faster race dogs (Mosher et al. (2007) PLOS Genetics 3:0779-0786). The deletion causes a premature truncation of the protein resulting in a loss of 17% of the carboxyl terminus. MSTN appears to be a negative regulator of skeletal muscle mass and is highly conserved in mammals.

Alternatively, a chromosome or chromosomes may be screened using comparative genomic hybridization (CGH; Pinkel & Albertson (2005) Nature Gen. 37:S11-S17). In this instance, reference “normal” genomic DNA and test genomic DNA are differentially labeled and hybridized to metaphase chromosomes or DNA microarrays. The relative hybridization signal at a given location is proportional to the relative copy number of the sequences in the reference and test genomes. Arrays may be generated using DNA obtained from, for example, bacterial artificial chromosomes (BACs) or PCR. BACs have been instrumental in genome sequencing and have been described for a wide variety of mammals, including dog, cat, horse, cow, pig, deer, elephant, non-human primates, and humans, for example.

### EXAMPLE 13

### Chromosome Isolation

A specific chromosome or chromosomes may be selected or alternatively eliminated based on screening a chromosome or chromosomes using the methods described herein. A specific chromosome or chromosomes may be isolated either before or after the selection process. A specific chromosome or chromosomes may be isolated by using fluorescence activated cell/chromosome sorting (FACS) (Gray et al. (1987) 238:323-329). Alternatively, a chromosome or chromosomes may be isolated using microdissection with a glass needle (Kao & Yu (1991) Proc. Natl. Acad. Sci. USA 88:1844-1848), optical tweezers (Ojeda et al. (2006) Optics Express 14:5385-5393), dielectrophoresis (U.S. Pat. No. 7,198,702), laser microdissection (Thalhammer et al. (2004) Chromosome Res. 12:337-343;), atomic force microscopy (Tsukamoto et al. (2006) Nanotechnol. 17:1391-1396; Kim et al. (2006) Curr. Applied Phys. 6:663-668), or magnetic beads (U.S. Pat. No. 5,508,164). Alternatively, a chromosome or chromosomes may be isolated using microcell-mediated transfer (Schultz et al. (1987) Proc. Natl. Acad. Sci. USA 84:4176-4179).

Under certain conditions, it may be optimal to sort one or more chromosomes from a cell population. FACS may be used for this purpose (see, e.g. Yu et al. (1981) Nature 293:154-155; Langlois et al. (1982) Proc. Natl. Acad. Sci. USA 79:7876-7880; Gray et al (1987) Science 238:323-329; Davies (1998) Proc. RMS 33:163-164). For these experiments, mitotic cells with condensed metaphase chromosomes are generated using colcemid to inhibit progress of cells through mitosis. The mitotic cells are swollen in a hypotonic buffer containing, for example, 50 mM KCl, 5 mM Hepes, 10 mM MgSO₄, 3 mM dithioerythritol, and 0.25% Triton X-100 and mechanically disrupted using needle shearing or vortexing. The released mitotic chromosomes are equilibrated with one or more DNA-specific fluorescent dyes such as, for example, propidium iodide and ethidium bromide (bind double-stranded nucleic acids with no base composition preference), Hoechst 33258 and 4,6-diamidino-2-phenylindole (DAPI; bind preferentially to adenine and thymine rich DNA), and chromomycin A3 and mithramycin (bind preferentially to guanine and cytosine rich DNA).

Once labeled, the chromosomes are sorted by FACS based on the intensity of the fluorescence staining which directly correlates with the size of a specific chromosome. For example, chromosomes labeled with Hoechst 33258 and chromomycin A3 flow sequentially through two laser beams, one adjusted to 458 mm to excite chromomycin A3 and the other adjusted to ultraviolet wavelengths 351 and 363 nm to excite Hoechst 33258. The Hoechst 33258 and chromomycin A3 content of each chromosome is determined by measuring the intensities of fluorescence and sorted chromosomes collected, resulting in a population enriched for chromosome 1 or chromosome 2, for example, relative to other chromosomes. This enriched population may then be used for chromosome selection using the methods described herein.

Optionally, specific populations of chromosomes may be identified and isolated using specific fluorescent chromosome paints. For example, chromosome I may be labeled with a chromosome I specific paint from, for example, CytoCell Technologies (Cambridge, UK), and selectively sorted using FACS. Alternatively, all but chromosome 1, for example, may be painted using chromosome specific paints and FACS used to eliminate the labeled chromosomes. In this manner, chromosome 1, for example, may be isolated in the absence of detecting dye. In either instance, the enriched chromosome population may be used for chromosome selection using the methods described herein. Similar approaches may be used to isolate other specific chromosomes.

In addition, isolation of a single targeted chromosome, either labeled or unlabeled, may be possible using nanotechnology versions of flow cytometry and dielectrophoresis (see, e.g., Leary (2005) Cytometry 67A:76-85; Zheng et al. (2006) Proceedings of 2006 International Conference on Microtechnologies in Medicine and Biology, IEEE, Okinawa, Japan, 9-12 May, 2006; Gao et al. (2003) Proceedings of the 25^{th} Annual International Conference of the IEEE EMBS, Cancun, Mexico, September 17-22, 3348-3351).

A chromosome or chromosomes may be isolated using microdissection using, for example, a glass microneedle (Kao & Yu (1991) Proc. Natl. Acad. Sci. USA 88:1844-1848). In this instance, cells may be treated with colcemid and swollen with a hypotonic buffer as described herein. The suspension of swollen cells are delivered drop-wise to a microscope slide from a height sufficient to break open the cells upon impact, creating a metaphase spread. Individual chromosomes in the metaphase spread may be identified under the microscope using standard cytometry techniques. Individual chromosomes are isolated from the metaphase spread using a micromanipulator and a glass microneedle.

Alternatively, a chromosome or chromosomes may be isolated using optical tweezers and Raman spectroscopy (Ojeda et al. (2006) Optics Express 14:5385-5393). As such, metaphase chromosomes are isolated from a cell or cells using the methods described herein, and placed in suspension in one of several wells on a microscope slide. Chromosomes 1, 2, and 3, for example, may be screened based on size and centromere location. The optical tweezers are used to trap and selectively move a chromosome or chromosomes to a separate well. In addition, each chromosome may be identified by its unique Raman spectroscopic profile (Ojeda et al. (2006) Optics Express 14:5385-5393). The sorted chromosomes may be collected from the wells for further processing. Alternatively, chromosomes may be sorted using optical tweezers in combination with one or more chromosome-selective fluorescent markers such as, for example, a labeled PNA or a chromosome paint as described herein.

A chromosome or chromosomes may be isolated using microcell-mediated cell transfer (see, e.g., Schultz et al. (1987) Proc. Natl. Acad. Sci. USA 84:4176-4179; Seyrantepe et al. (2006) Hum. Genet. 120:293-296; Kugoh et al. (1999) DNA Res. 6:165-172; U.S. Patent Application 2006/0166257 A1). In this instance, cells are treated with 0.05 μg/ml colcemid for 48 hours to form micronuclei followed by digestion with 10 μg/ml cytochalasin B and centrifugation to form microcells. The microcells formed in this manner may contain one or more chromosomes. As such, the chromosomes in the microcells may be screened non-destructively, for example, using a tagged PNA probe as described herein. Microcells containing one or more chromosomes may be fused with a second cell in metaphase, facilitating the transfer of one or more chromosomes from one cell to another. Optionally, the donor cells may be transfected with pSV2bsr, for example, which incorporates into the donor cell DNA and may be used for clonal selection in the presence of blastcidine S hydrochloride following microcell transfer (Kugoh et al. (1999) DNA Res. 6:165-172). Alternatively, the donor cell chromosomes may be tagged with pSVneo and selected with G418 (Schultz et al. (1987) Proc. Natl. Acad. Sci. USA 84:4176-4179).

Optionally a chromosome may be isolated using cloning techniques. Methods have been developed to amplify whole genomes using multiple displacement amplification or MDA (see, e.g. Dean et al. (2002) Proc. Natl. Acad. Sci. USA 99:5261-5266). The technique is based on rolling circle amplification which was developed for amplifying large circular DNA templates. For these experiments, chromosomal DNA is amplified at 30° C. without a thermocycler using Phi29 DNA polymerase and random exonuclease resistant hexamer primers (see, e.g. GenomiPhi™ DNA Amplification Kit, GE Healthcare Life). Phi29 DNA polymerase has great processivity, allowing for synthesis of DNA strands as long as 70 kb in length. Similar techniques are currently being used to do whole genome amplification from human cells, routinely generating fragments >10 kb (Dean et al. (2002) Proc. Natl. Acad. Sci. USA 99:5261-5266). The genomic DNA from a single sperm, for example, may be amplified using this approach (Jiang et al. (2005) Nucleic Acids Res. 33:e91). An intact full-length chromosome may be reconstructed using this approach.

A mammalian chromosome may also be isolated using a bacterial artificial chromosome (BAC) library. For example, comprehensive coverage of the human and bovine genomes with a BAC library have been reported (Osoegawa et al. (2001) Genome Res. 11:483-496; Eggen et al. (2001) Genet. Sel. Evol. 33:543-548). BACs are capable of carrying approximately 300 kb of inserted DNA sequence (Shizuya et al. (1992) Proc. Natl. Acad. Sci. USA 89:8794-8797). Alternatively, a mammalian chromosome may be isolated using yeast artificial chromosomes (YACs) which can carry approximately 500 kb of foreign DNA. As such, an intact full-length chromosome may be reconstructed using this approach.

Optionally, an artificial mammalian chromosome may be isolated or engineered. For example, a human artificial chromosome (HAC) is a microchromosome (6-10 Mb) that can act as a new chromosome in a population of human cells. Artificial mammalian chromosomes in the form of minichromosomes and satellite artificial chromosomes have also been described (U.S. Pat. No. 6,025,155). An artificial mammalian chromosome may be generated using a top down methodology in which endogenous chromosomes are fragmented at their ends by trimming off the telomeres, for example (Wong et al. (2005) J. Biol. Chem. 280: 3954-3962; Willard (2001) Proc. Natl. Acad. Sci. USA 98:5374-5376). Alternatively, an artificial mammalian chromosome may be generated using a bottom up approach in which specific genes ranging in size from 100-500 kb are subcloned (see, e.g. Suzuki et al. (2006) J. Biol. Chem. 281:26615-26623). A HAC, for example, appears to retain most of the functions expected of the centromere of a stable chromosome and to align at the metaphase plate accurately (Tsuduki et al. (2006) Mol. Cell. Biol. 26:7682-7695).

### EXAMPLE 14

### Chromosome Selection from Sperm

Sperm cells from, for example, boar, bull, stallion or ram, are collected using known animal husbandry methods including using a gloved-hand, an artificial vagina, and/or electro-ejaculation methods as appropriate.

After collection, the semen is diluted with a species-specific buffer to extend the lifespan of the sperm outside the body (e.g. artificial insemination buffer). Appropriate diluents provide energy and nutrients, buffering action for pH changes (e.g. due to lactic acid formation), protection from temperature shock (e.g. rapid shock), maintain osmotic pressure, balance electrolytes, inhibit microorganism growth, as well as facilitating dilution to an appropriate volume for hybridization and selection. For example, a 2.9% sodium citrate—egg yolk buffer may be used for cattle (see, e.g., J. Dairy Sci. (1941) 24:905), and Beltsville Thaw Solution (BTS) may be used for boar sperm.

Optionally, sperm may be lysed prior to hybridization using a sperm lysis buffer consisting of 0.01 M Tris-HCl, pH 8.0, 0.01 M EDTA, 0.1 M NaCl, 2% SDS, and 20 μg/ml proteinase K (El Maarri et al. (2001) Nature 27:341-344. Sperm may be incubated in lysis buffer at 37° C. to 55° C. for 1 to 16 hours and optionally followed by deactivation of proteinase K at 95° C. for 10 minutes (Katoh et al. (2005) Exp. Anim. 54:373-376).

Optionally, sperm DNA may be condensed into a metaphase state to facilitate hybridization and chromosome isolation. Condensation may be induced, for example, by incubation of the sperm with hamster ova (Kamiguchi et al. (1986) Am. J. Hum. Genet. 38:724-740) or Xenopus laevis egg extract (Kimura et al. (2001) J. Biol. Chem. 276:5417-5420). Optionally, condensation may be induced by incubation with enucleated mouse oocytes, as described by Araki et al. (Romanato et al. (2005) Human Reprod 20:2784-2789). Briefly, mouse oocytes are isolated following superovulation induced by intraperitoneal injection of 5 IU pregnant mares serum gonadotrophin (PMSG) and human chorionic gonadotropin (hCG). The oocytes are freed from cumulus cells by pipetting in, for example, human tubal fluid supplemented with synthetic serum substance (e.g. Complete HTF medium with SSS™, Irvine Scientific, Santa Ana, Calif.). The isolated oocytes are incubated with 5 μg/ml cytochalasin B for 10 minutes at 37° C. and the metaphase II chromosome-spindle complex are aspirated into a pipette with minimal loss of oocyte cytoplasm. Sperm are introduced into the enucleated mouse oocytes by intracytoplasmic sperm injection using standard procedures. Sperm metaphase chromosomes may be isolated 15 to 16 hours after injection by lysis of the oocyte. As such, sperm chromosomes in metaphase may be screened for selection using the methods described herein.

### EXAMPLE 15

### Chromosome Selection from Spermatogenic Cells

Spermatogenic or male germ cells may be isolated from the testes of a mammal and used for chromosome selection. Spermatogonial stem cells represent a small population of self-renewing cells within the testes that are capable of undergoing spermatogenesis to form sperm throughout the lifetime of a male mammal (Brinster (2002) Science 296:2174-2176). Spermatogenesis can be divided into three phases: mitotic expansion of spermatogonia, meiotic diversification in spermatocytes, and maturation into spermatozoa to acquire mobility. At the corner stone of this process are the spermatogonial stem cells which are self-renewing and thought to support spermatogenesis throughout an animal's lifetime.

Spermatogonial stem cells may be isolated from an adult testis, for example, using multiple needle biopsy samples (see, e.g., Tesarik et al (2000) Hum. Reprod. 15:1350-1354). Tissue isolated in this manner is immediately placed in a medium containing Eagle's balanced salt solution, 75 mg/l penicillin, 11 mg/l pyruvic acid, 10 mg/l human serum albumin, and Hepes and minced using two sterilized microscope slide. The dispersed cells are cultured in the medium described above in the presence of 25 mIU/ml recombinant human follicle stimulating hormone (FSH). Under these conditions, a subset of primary and secondary spermatocytes differentiate into haploid round and elongated spermatids within 24 hours.

Spermatogonial stem cells may be enriched from adult testes using an anti-Thy1 strategy. Thy-1 is specifically expressed on spermatogonial stem cells, for example, in the mouse (Kubota et al. (2004) Cell Biology 47:16489-16494). Dissociated cells from an adult testis are incubated with a biotinylated antibody to Thy-1 (from, for example, BD Biosciences, Franklin Lakes, N.J.) and subsequently incubated with a streptavidin conjugated fluorescent label, such as Alexa Fluor 488-SAv (from, for example, Molecular Probes, Eugene, Oreg.). The dissociated testis cells are sorted, for example, on a FACStar Plus instrument (BD Biosciences) equipped with a Coherent Enterprise II laser (488 nm) and an air-cooled helium neon laser (633 nm). Cells may be sorted into sterile tubes containing, for example, phosphate buffered saline supplemented with 10% fetal bovine serum, 10 mM HEPES, 1 mM pyruvate, antibiotics, and 1 mg/ml glucose. Alternatively, spermatogonial stem cells may be enriched from adult testes using magnetic microbeads conjugated to an anti-Thy-1 antibody (e.g. 30-H12, Miltenyi Biotec, Gladbach, Germany) as described by Kubota et al. (Biol. Reprod. (2004) 71:722-731).

Alternatively, spermatogonial stem cells may be isolated from the neonate testes, for example, of bovine, rat or mouse (Lee et al. (2001) Biol. Reprod. 65:873-878; Tres & Kierszenbaum (1983) Proc. Natl. Acad. Sci. USA 80:3377-3381; Kanatsu-Shinohara et al. (2005) Biol. Reprod. 72:236-240; U.S. Patent Application 2006/0265774 A1). Spermatogonial stem cells isolated, for example, from a mouse neonatal testis may be expanded in vitro in the presence of glial cell line-derived neurotrophic factor (GDNF), leukemia inhibitory factor (LIF), epidermal growth factor (EGF), and basic fibroblast growth factor (bFGF) on mitomycin C-inactivated mouse embryonic feeder cells. After 2 years in continuous culture, these cells maintain the capacity to undergo spermatogenesis after transplantation into irradiated testis (Kanatsu-Shinohara et al. (2005) Development 132:4155-4163).

Isolation of spermatogenic cells from bovine testes, for example, begins with dissection of the testes (Lee et al. (2001) Biol. Reprod. 65:873-878). The testes of a 3 day old bull, for example, is decapsulated and 1 to 5 grams of the exposed parenchyma is removed, cut into small pieces and washed for 20 minutes in phosphate buffered saline free of calcium and magnesium (Ca²⁺, Mg²⁺-free PBS). The tissue is dissociated in a buffer containing, for example, 0.5 mg/ml collagenase, 10 μg/ml DNase 1, 1 μg/ml soybean trypsin inhibitor and 1 mg/ml hyaluronidase in Ca²⁺, Mg²⁺-free PBS at room temperature for 30 minutes. The dissociated tissue is spun at 400×g to remove peritubular cells. The resulting pellet is digested for an additional 30 minutes in the buffer described above with collagenase increased to 5 mg/ml. The digest is spun at 600×g for 10 minutes and the resulting cell pellet washed several times in Dulbecco modified Eagle medium/F12 (DMEM/F12) and immediately put into culture. Similar procedures are described for isolating spermatogenic cells from neonate rat and mouse testes (Tres & Kierszenbaum (1983) Proc. Natl. Acad. Sci. USA 80:3377-3381; Kanatsu-Shinohara et al. (2005) Biol. Reprod. 72:236-240). Alternatively, spermatogonial stem cells may be isolated from neonatal or juvenile testes using the Thy-1 isolation strategy described herein.

The isolated spermatogenic cells may be cultured as a dispersed population, either with or without a feeder cell layer, for example, of mouse embryonic fibroblasts (Kanatsu-Shinohara et al. (2005) Biol. Reprod. 72:236-240). In the absence of a feeder layer, spermatogenic stem cells may be cultured on laminin coated plates, for example, in a medium containing, for example, StemPro-34 SFM (Invitrogen, Carlsbad, Calif.) supplemented with StemPro Supplement (Invitrogen, Carlsbad, Calif.), 25 μg/ml insulin, 100 μg/ml transferrin, 60 μM putrescine, 30 nM sodium selenite, 6 mg/ml glucose, 30 μg/ml pyruvic acid, 1 μl/ml DL-lactic acid, 5 mg/ml bovine serum albumin, 2 mM L-glutamine, 5×10⁻⁵ 2-mercaptoethanol, MEM Vitamin Solution (Invitrogen), MEM non-essential amino acids solution (Invitrogen), 10-4 ascorbic acid, 10 μg/ml D-biotin, 30 ng/ml β-estradiol and 60 ng/ml progesterone, and 0.1-10% bovine calf serum as described by Kanatsu-Shinohara et al (2005). The medium may be further supplemented with growth factors including 20 ng/ml EGF, 10 ng/ml bFGF, 10³ U/ml ESGRO (a murine leukemia inhibitory factor, Invitrogen), and 10 ng/ml recombinant rat GDNF (R&D Systems, Minneapolis, Minn.).

Alternatively, the disaggregated spermatogenic cells may be reaggregated to promote close association of germ cells with, for example, Sertoli cells. Reaggregation may be accomplished, for example, by encapsulating the cells in alginate (Lee et al. (2001) Biol. Reprod. 65:873-878; U.S. Pat. No. 6,872,569 B2). Disaggregated cells are treated with 100 μg/ml phytohemagglutinin for 10 minutes at 32° C. to induce cell aggregation, spun at 600×g and the resulting pellet treated with 1% sodium alginate and 0.9% NaCl. The aggregated-alginate treated cells are extruded through a pipette into a tissue culture plate containing 1.5% CaCl₂ and 0.9% NaCl, inducing solidification. The alginate encapsulated cells are cultured in Hepes-buffered DMEM/F12 supplemented with 10 μg/ml insulin-transferrin-selenium solution (Invitrogen), 10⁻⁴ M vitamin C, 10 μg/ml vitamin E, 3.3×10⁻⁷ retinoic acid, 3.3×10⁻⁷ retinol, 1 mM pyruvate, 2.5×10⁻⁵ IU FSH, 10⁻⁷ M testosterone, penicillin/streptomycin and 10% bovine calf serum.

Spermatogenic cells in the diploid state may be used for chromosome selection. Alternatively, spermatogonia may be differentiated in vitro through meiosis and chromosome selection performed on haploid cells as described herein for isolated sperm. In the case of encapsulated spermatogenic cells from neonate bovine, haploid cells indicative of spermatogenesis are observed from 5 to 14 weeks in culture.

### EXAMPLE 16

### Chromosome Selection from Female Oocytes

A chromosome or chromosomes may be selected from a mature female reproductive cell, including oocytes, ova, and/or polar bodies. Oocytes from, for example, cows, sows, ewes, and mares are collected using known animal husbandry methods including, for example, super-ovulation, in vitro production, and collection at slaughter. Mice, for example, may be super-ovulated by consecutive injections of eCG (5 IU) and hCG (5 IU) 48 hours apart. About 14 hours following hCG injections, oocyte-cumulus complexes are released from oviducts into Hepes-CZB. Cumulus cells can be dispersed by 5 minutes treatment with 0.1% bovine testicular hyaluronidase (300 USP units/mg; from, for example, ICN Pharmaceuticals, Costa Mesa, Calif.) in Hepes-CZB (see, e.g., Wakayama et al. (1998) Biol. Reprod. 59:100-104).

After collection, female reproductive cells may be maintained in a variety of balanced salt solutions (e.g. TC199, M16, NCSU23) known in the art at an appropriate temperature resembling, for example, the body temperature of the species from which the cell was isolated (e.g. mice at 37° C., pig at 39° C.). Cumulus-free mice oocytes can be kept in CZB at 37.5° C. under 5% CO₂ in air. Appropriate solutions and temperatures extend the length of cell viability and function and may be modified as appropriate (see, e.g., Goodall et al. (1986) J. Cell Biol. 102:568-575).

Screening of oocyte chromosomes may be done in intact cells as described herein and as described, for example, in Pellestor et al. (2005) Hum. Reprod. Update 11:15-32. Alternatively, the oocyte nuclei and the associated polar body may be isolated together or separately and the associated chromosomes screened. Methods of designing and constructing probes to bind and/or hybridize to target DNA sequences such as those indicative of a particular allele, trait locus, or other feature of interest are known in the art and described herein. Probes may include peptide nucleic acids, polyamides, and/or oligonucleotides, among others, and may be tagged with one or more tags known in the art and/or described herein.

### EXAMPLE 17

### Chromosome Selection from Female Primordial Cells from Ovaries

A chromosome or chromosomes may be selected from oocytes derived from in vitro differentiated/oogenesis of ovarian follicles. Follicle culture systems have been described for a variety of mammalian species, including human, mouse, rat, hamster, pig, bovine, and baboon (see, e.g., Salha et al. (1998) Hum. Reprod. Update 4:816-832).

For example, granulosa cells and oocytes may be derived in vitro through differentiation of ovarian surface epithelium (Bukovsky et al. (2005) Reprod. Biol. Endocrinol. 3:17). In this instance, epithelial cells are scraped from the surface of an ovary and grown in culture medium with or without estrogenic stimuli. In the absence of estrogenic stimuli, a subset of the cells differentiate into granulosa cells. In the presence of estrogenic stimuli, a small number of large cells with oocyte phenotype (120-180 μm in diameter) are observed after 5-6 days in culture. The large cells may also contain two nuclei, one of which stains positively with an antibody to zona pellucida proteins. The presence of the second unstained nuclei is indicative of a polar body, suggesting that the cells have undergone the first meiotic division. The later may be verified, for example, using an antibody (PSI) to a carbohydrate zona pellucida antigen specifically expressed during meiosis.

Alternatively, oocytes may be derived in vitro from non-growing, early stage oocytes from early antral ovarian follicles (Katska-Ksiazkiewicz (2006) Reprod. Biol. 6:3-16). For example, 130,000 to 235,000 non-growing and growing oocytes are enclosed in ovarian follicles of a cow, but only a small percentage proceed in vivo through full maturation and ovulation. As such, bovine follicles, for example, may be isolated by microdissection of ovarian slices. Either small intact follicles (0.2 to 0.5 mm diameter) or larger cumulus-oocyte complexes with associated granulosa (0.4 to 0.7 mm in diameter) may be used for culturing. Follicles may be grown, for example, in TCM 199 supplemented with 3% bovine serum albumin and 4 mM hypoxanthine either on or embedded in a collagen gel (Katska-Ksiazkiewicz et al. (2006) Reprod. Biol. 6:21-36). The collagen gel may prevent migration of granulosa cells away from the maturing oocytes, the later of which is dependent on granulosa cells for growth and development (Salha et al. (1998) Hum. Reprod. Update 4:816-832). After 7 to 10 days in culture, a subset of cells may have reached meiotic competence and fertilizability. Optionally, fatty acid-free bovine serum albumin and/or PVP40 may be added to the culture to enhance meiotic competence and fertilizability.

Alternatively, oocytes may be derived in vitro from premeiotic female germ cells (Obata et al. (2002) Nature 418:497-698). In this instance, developing ovaries, from for example, a 12.5 day post coitum mouse fetus, are isolated and cultured for 7 days in the presence of the mesonephroi, and for 10 days in the absence of the mesonephroi. Secondary follicles are isolated and further cultured for 11 days. As these cells do not resume meiosis, the nuclei are transferred to fully grown adult oocytes, at which point meiosis and maturation to metaphase continue.

### EXAMPLE 18

### Chromosome Selection from Germ Line Stem Cells

A chromosome or chromosomes may be selected from a germ line stem cell. A germ line stem cell may be derived from primordial germ cells (PGCs) arising from the embryonal ectoderm in the mouse, for example, at 7 to 7.5 days of gestation (Brinster (2002) Science 296:2174-2176). Similarly, PGCs may be isolated, for example, from porcine fetuses from the urogenital ridge at day 25-27 (Piedrahita et al. (1998) Biol. Reprod. 58:1321-1329). Alternatively, a germ line stem cell may be derived from an embryonic stem cell, for example (see, e.g., Aflatoonian & Moore (2006) Reproduction 132:699-707). Embryonic stem cells are derived from mammalian preimplantation blastocysts and have the ability to self-renew indefinitely or to differentiate under certain culture conditions into a wide range of cell types. Alternatively, a germ line stem cell may be derived from a postnatal tissue such as, for example, bone marrow (see, e.g., Drusenheimer et al. (2007) Soc. Reprod. Fertil. Suppl. 63:69-76). As such, conditions are used to differentiate a stem cell down either a male germ cell lineage or a female germ cell lineage, as appropriate.

Male germ cells and haploid gametes for chromosome selection may be derived from embryonic stem cells using published protocols (see, e.g. West et al. (2006) Nat. Protocols 1:2026-2036). Primordial germ cells arise in vivo from the proximal epiblast. Embryonic stem cells differentiate in vitro into cystic structures called embryoid bodies consisting of tissue lineages typical of the early mouse embryo. As such, it is possible to isolate putative primordial germ cells (PGCs) from the embryoid bodies based on the relative expression of specific markers such as SSEA, Oct4, Gcnf, Piwil2, and Dazl, for example, and on proliferation of the PGCs in response to retinoic acid (Geijsen et al. (2004) Nature 427:148-154). Continued growth of PGCs in culture leads to a subset of cells that have undergone meiosis as judged by staining with an antibody, FE-J1, that specifically recognizes male meiotic germ cells (Geijsen et al. (2004) Nature 427:148-154).

Alternatively, male germ cells for chromosome selection may be derived from differentiation of adult bone-marrow-derived mesenchymal stem cells (Drusenheimer et al. (2007) Soc. Reprod. Fertil. Suppl. 63:69-76). For example, mesenchymal stem cells from the bone marrow are aspirated from an adult male, for example, and mixed with Dulbecco's Modified Eagle Medium (DMEM) and fractionated on a Ficoll-Hypaque density gradient to isolate a low-density mononuclear fraction. Prior to differentiation, the isolated cells are cultured in a mesenchymal stem cell culture medium such as, for example, MesenCult Basal Medium with mesenchymal stem cell supplements (from, for example, StemCell Techonolgies, Vancouver, Canada). To induce differentiation, the mesenchymal stem cells are cultured for fifteen days in RPMI 1640 (Invitrogen, Carlsbad, Calif.) in the presence of retinoic acid at a final concentration of 1-10 μM, at which point several markers of germ cells are upregulated including Dazl, Piwil2, Stra8 and Tspy (Drusenheimer et al. (2007) Soc. Reprod. Fertil. Suppl. 63:69-76).

Female germ cells and oocytes for chromosome selection may be derived from embryonic stem cells (Hubner et al. (2003) Science 300:1251-1256; West et al. (2006) Nature Protocols 1:2026-2036). Embryonic stem cells from mice, for example, are grown in ES medium with heat-inactivated serum in the absence of feeder cells or the growth factors required for self-renewal. As such, the cells may proceed down various differentiation paths. At day 7, cells may be sorted based on expression levels of Oct4, a germ line specific gene and further assessed for expression of other germ line markers, including, for example c-kit, Vasa, synaptonemal complex protein 3 (SCP3) and meiosis-specific homologous recombination gene (DMC1) (Hubner et al. (2003) Science 300:1251-1256). After further culturing for 16 to 20 days, follicle-like structures may begin to form and as early as 26 days of culture, oocyte-like cells that have under gone the first phase of meiosis may become apparent.

Female germ cells may be derived from adult bone-marrow-derived mesenchymal stem cells as bone marrow grafts into female mice, for example, produces new follicles and oocytes in the recipient ovary (Johnson et al. (2004) Nature 428:145-150).

### EXAMPLE 19

### Chromosome Selection from Somatic Cells of One or Multiple Donors

A chromosome or chromosomes may be selected from a somatic cell or cells of one or more individuals. For example, chromosomes may be isolated from peripheral lymphocytes (Langlois et al. (1982) Proc. Natl. Acad. Sci. 79:7876-7880). Lymphocytes are isolated using standard procedures. Briefly, peripheral blood mononuclear cells (PBMCs) are isolated from EDTA-treated donor blood by centrifugation through Ficoll-Hypaque Plus (Amersham Biosciences, Piscataway, N.J.), and incubated for 3 hours at 37° C. in RPMI 1640 medium supplemented with 10% fetal bovine serum (FBS), 50 units/ml penicillin, and 50 mg/ml streptomycin. After 3 hours, the non-adherent lymphocytes are removed, optionally stimulated with phytohemagglutinin, and further cultured in fresh medium for an additional 2-4 days. The cells are then treated with colcemid (0.2 μg/ml) in culture medium for 10 hours to generate condensed metaphase chromosomes. The cells are swollen in a hypotonic solution containing 75 mM KCl, resuspended in isolation buffer containing 25 mM Tris, pH 7.5, 0.75 M hexylene glycol, 0.5 mM CaCl₂, and 1 mM MgCl₂, and forced through a 22 gauge needle to release the chromosomes from the cells and into suspension. The isolated chromosomes may be screened using the methods described herein.

### EXAMPLE 20

### Transferring Chromosomes Into a Cell for Propagation

A selected chromosome or chromosomes may be transferred into a somatic or germ cell line for propagation. As such, an entire genome from a selected cell, such as for example, a selected sperm cell, may be transferred to a cell line for propagation. Alternatively, a selected chromosome or chromosomes may be added to an existing somatic or germ line cell genome, such that the selected chromosome replaces an endogenous, non-optimal chromosome. Alternatively, a selected chromosome or chromosomes may be added to a cell line from another species to generate a hybrid cell from which the selected chromosome or chromosomes may be isolated in the future (for example human/mouse hybrids).

Undesired chromosomes may be eliminated from a cell or cells prior to addition of a selected chromosome or chromosomes. Alternatively, undesired chromosomes may be eliminated after the addition of a selected chromosome or chromosomes. Undesired chromosomes may be eliminated using, for example, live-cell laser ablation (see, e.g., Stark et al. (2003) Eur. Biophys. J. 32:33-39). As such, cells are treated with colcemid to generate metaphase chromosomes as described herein. A specific chromosome or chromosomes may be irradiated, for example, with a UV laser microbeam (from, for example, PALM Microlaser Technologies, Bernried, Germany). The UV laser may be a pulsed nitrogen UV laser (wavelength 337.1 nm) with a pulse width, for example of less than 4 nanoseconds, a pulse energy of 300 uJ and a pulse repetition rate between 0 and 60 Hz and laser cutting width of 380 nm (Stark et al. (2003) Eur. Biophys. J. 32:33-39). Laser ablation may be combined, for example, with atomic force microscopy to provide high-resolution imaging and precise mechanical manipulation capabilities (Stark et al. (2003) Eur. Biophys. J. 32:33-39).

Alternatively, laser ablation of a chromosome or chromosomes may be performed using two photon laser ablation in the presence of a photosensitizing dye (Fischer et al. (2007) J. Opt. A: Pure Appl. Opt. 9:S19-23; Berns et al. (2000) Proc. Natl. Acad. Sci. USA 97:9504-9507). For example, ethidium monoazide bromide is a DNA intercalating dye which upon exposure to light at a wavelength, for example, of 488 nm, covalently binds to the DNA and inactivates associated genes. As such, cells may be incubated with 10 μg/ml ethidium monoazide bromide for 12 hours followed by exposure to focused light. Alternatively, a cell treated with ethidium monoazide bromide may be irradiated with focused 1 μm diameter beam from a 100 ps pulsed Nd-Yag laser (1.06 um wavelength) operating at 70 MHZ (Berns et al. (2000) Proc. Natl. Acad. Sci. USA 97:9504-9507). Alternatively, a cell or cells may be incubated with the photosensitizer methylene blue, and a chromosome or chromosomes irradiated using two-photon ablation with 100 fs laser pulses at a wavelength of 1278 nm emitted from a Cr:forsterite laser (Fischer et al. (2007) J. Opt. A: Pure Appl. Opt. 9:S19-23).

A selected chromosome or chromosomes may be microinjected into a somatic or germ line cell. For example, somatic cells in a mitotic state may be microinjected with a biological material such as a chromosome, for example, using a microinjector (from, for example, Narishige International USA, Inc., East Meadow, N.Y.) and using an injection volume of up to 5% of the cellular volume (Cambell & Gorbsky (1995) J. Cell. Biol. 129:1195-1204). Similarly, an oocyte at metaphase II, for example, may be injected with a chromosome or chromosomes.

A selected chromosome or chromosomes may be transferred into a recipient cell using microcell-mediated transfer (see, e.g., Schultz et al. (1987) Proc. Natl. Acad. Sci. USA 84:4176-4179; Seyrantepe et al. (2006) Hum. Genet. 120:293-296; Kugoh et al. (1999) DNA Res. 6:165-172). The recipient cell may be, for example, a somatic cell or a proliferating germ line cell. As such, microcells are generated using colcemid and cytochalasin as described herein. The purified microcells containing one or more chromosome are centrifuged at 400×g for 10 minutes and resuspended in serum-free DMEM culture medium containing 100 μg/ml of phytohemagglutinin (PHA). The microcells are incubated with recipient cells for 15-20 minutes at which time a 45-50% PEG solution is added to facilitate cell fusion. The PEG solution is removed after 1 minute and the cells cultured in standard culture medium. The donor chromosomes may be optionally tagged with pSV2bsr or pSVneo and selected for using blastcidine S hydrochloride (BS) or G418, respectively, as described herein. As such, cells are treated for 3-4 weeks with medium containing BS or G418, killing those recipient cells lacking chromosomes transferred from the microcells. This method may also be used to isolate a haploid chromosome or chromosomes, forming monoallelic hybrids (see, e.g. U.S. Patent Application 2006/0166257 A1; Yan et al. (2000) Nature, 403:723-724).

Alternatively, a chromosome or chromosomes may be transferred into a cell using electroporation and optionally linked analysis by FACS to ensure proper insertion of chromosome (see, e.g., U.S. Patent Application 2002/0019052 A1).

Alternatively, a metaphase chromosome or chromosomes may be trapped in a phospholipid vesicle and subsequently transferred to a cell via fusion (Mukherjee et al. (1978) Proc. Natl. Acad. Sci. USA 75:1361-1365). For example, unsaturated phosphatidylcholine/cholesterol lipochromosomes may be generated, for example, using a molar ratio of 7:2 (egg lecithin/cholesterol). As such, the egg lecithin and cholesterol are combined in a round-bottom glass flask with the chromosomes suspended in ether/chloroform. Rotary evaporation at 37° C. is used to dry the mixture down to a thin film. The addition of buffered saline to the thin film results in formation of phospholipid vesicle encapsulating the chromosomes. The chromosomes within the vesicles may be screened and selected using the methods described herein. Alternatively, chromosomes may be detected in the vesicles using general DNA stains such as, for example, ethidium bromide or acridine orange (Mukherjee et al. (1978) Proc. Natl. Acad. Sci. USA 75:1361-1365). Fusion of vesicles with a recipient cell may be carried out using brief exposure to polyethylene glycol as described herein.

Alternatively, it may be desirable to replicate a chromosome or chromosomes in vitro using a cell free system. For example, replication of isolated sperm DNA or sperm DNA within an isolated nucleus may be initiated using a cell-free extract derived from Xenopus eggs (Hutchison et al. (1988) Development 103:553-566; U.S. Pat. No. 5,780,230). The Xenopus egg contains an excess of building blocks required to assemble chromatin and nuclei and consequently, incubation of enucleated sperm DNA with egg extract leads to formation of a “nucleus” in which replication takes place (Sheehan et al. (1988) J. Cell Biol. 106:1-12). Replication in egg extract appears to be dependent upon DNA assembly into nuclei containing nuclear lamins and functional nuclear pores. Once formed, the nuclear envelope appears to be instrumental in regulating the onset of S phase, apparently by regulating access of chromosomal DNA to one or more initiation factor from the cytoplasm (Cox (1992) J. Cell Sci. 101:43-53; Gilbert et al. (1995) Mol. Cell. Biol. 15:2942-2954). Replication of mammalian chromosomes in Xenopus egg extract may be dependent upon presence of mammalian nuclear factors (Gilbert et al. (1995) Mol. Cell. Biol. 15:2942-2954). Alternatively, chromosomes may be replicated in a human cell free system (Krude (2006) Cell Cycle 5:2115-2122).

The selected chromosomes may be inserted into separate cells such that each cell only has a single extra chromosome (2n+1). Alternatively, various combinations of selected chromosomes, optimized for a single or multiple traits, for example, may be inserted into a single cell. Monosomic cell lines in which one chromosome is haploid (2n−1) have been described and remain stable in culture (Clarke et al. (1998) Proc. Natl. Acad. Sci. USA 95:167-171).

In the instance in which the entire genome is haploid, for example, it may be of benefit to generate a diploid genome. Diploidization of a haploid oocyte genome, for example, may be performed by fertilizing an oocyte, selectively aspirating the male pronucleus and treating the oocyte with cytochalasin D at 0.33-0.5 μg/ml for 14 to 20 hours (see, e.g. Anderegg et al. (1986) Proc. Natl. Acad. Sci. USA 83:6509-6513; U.S. Patent Application 2006/0212948 A1; U.S. Patent Application 2007/0141702 A1). Embryonic stem cells containing the parthogenic genome may be isolated from developing embryos.

In some instances it may be beneficial to maintain a haploid genome. Haploid male embryos, for example, may be generated by enucleating metaphase II oocytes and inserting a male sperm head. Haploid embryonic stem cells may be generated from the resulting embryos (see, e.g., U.S. Patent Application 2004/0146865 A1). A diploid male embryo may be generated by inserting two male sperm heads using the same procedure (Latham et al. (2002) Biol. reprod. 67:386-392). Assuming that the selected chromosome or chromosomes of the two sperm heads are identical, this may lead to generation of identical chromosomes or alleles.

### EXAMPLE 21

### Transferring Optimized Chromosomes Into Germ Lines for Fertilization

An individual optimized chromosome or chromosomes may be transferred to a germ line cell for fertilization using the methods described herein. Alternatively, an entire optimized genome may be transferred from, for example, a self propagating cell line into a germ line cell. The germ line cell may be fully mature, having completed the cross-over events associated with meiosis and competent for fertilization such as, for example, a spermatid, a sperm or a mature oocyte. Alternatively, the germ line cell may be an embryonic stem cell, a primordial germ line cell, or a spermatogonial cell with the potential to differentiate into a sperm or oocyte competent for fertilization.

A diploid genome from a self-propagating cell line, for example, may be induced to form a haploid genome by artificial haploidization. Artificial haploidization of somatic nuclei, for example, may be accomplished using enucleated oocytes (see, e.g. Heindryckx et al. (2004) Hum. Reprod. 19:1189-1194). For example, a G2-stage somatic nucleus (diploid, double-chromatid chromosomes, 4c DNA) may be transferred into an immature germinal vesicle stage oocyte and induced to undergo reductional division in the absence of recombination and meiosis until metaphase 11-like arrest in order to extrude half of the somatic cell chromosomes into the first polar body. The artificial oocyte may be activated to enter metaphase II in which half of the remaining somatic cell chromosomes are extruded into the second polar body and a haploid genome remains in the ooplasm. Alternatively, an enucleated metaphase II oocyte may induce a G0/G1-stage somatic nucleus into a premature M-phase without previous S-phase, resulting in a pseudo-second polar body and a haploid set of somatic chromosomes in the reconstructed oocyte. As such, the haploid genome may be transferred into an enucleated spermatid, sperm or oocyte for fertilization.

A selected chromosome or chromosomes may be inserted by microinjection into a mature oocyte in which the endogenous chromosome or chromosomes has been ablated using the methods described herein. The modified oocyte containing a selected chromosome or chromosomes may be fertilized by donor sperm, for example, following standard procedures. Alternatively, an entire optimized genome from a somatic cell or germ line cell may be transferred into an oocyte using well established methods of nuclear transfer (see, e.g., Wakayama et al. (1998) Nature 394:369-374; Wakayama (2007) J. Reprod. Develop. 53:13-26). As such, the cell or nuclei may be directly microinjected into an oocyte in which the metaphase II chromosomes, for example, have been removed. Alternatively, an unactivated oocyte may be used for nuclear transfer (see, e.g., U.S. Patent Application 2007/0028312 A1). Alternatively, the cell may be inserted into the perivitelline space and fused with the enucleated oocyte using a brief electrical pulse. The entire optimized genome may be haploid, in which case the modified oocyte may be fertilized by donor sperm, for example, following standard procedures. Alternatively, the entire optimized genome may be diploid in which case the modified oocyte may proceed into embryogenesis.

A selected chromosome or chromosomes may be reinserted into a donor sperm in which the complimentary chromosome or chromosomes has been ablated, for example, by laser ablation. Alternatively, the chromosome or chromosomes may be inserted into the male pronucleus post fertilization, in which case the complimentary chromosome or chromosomes have been ablated either prior to fertilization or post fertilization.

A selected chromosome or chromosomes may be incorporated into an embryonic stem cell, a primordial germ line cell, or a spermatogonial cell using the methods described herein. The selected chromosome or chromosomes may be haploid. Alternatively, the selected chromosome or chromosomes may be diploid and isogenic, for example. In some instances, a nucleus containing an optimized genome may be transferred into a proliferating germ line cell. As such, the nucleus may be transferred into an embryonic stem cell, a primordial germ line cell, or a spermatogonial cell.

The recipient cell may be enucleated using cytochalasin B in combination with centrifugation. For example, cells may be treated with cytochalasin B at a concentration of 4-6 μg/ml in culture medium for 12-16 hours, at which time the nuclei protrude from the cytoplasm (Ladda & Estensen (1970) Proc. Natl. Acad. Sci. 67:1528-1533). Centrifugation may be used to isolate the nuclei or karyoplast from the enucleated cell or cytoplast (Shay (1977) Proc. Natl. Acad. Sci. USA 74:2461-2464).

A nucleus may be transferred to the cytoplast using microinjection techniques. Alternatively, karyoplasts containing only the nuclei may be generated using the methods described here in and used for fusion with a cytoblast, for example, using polyethylene glycol (Shay (1977) Proc. Natl. Acad. Sci. USA 74:2461-2464). Alternative methods for fusing karyoplasts and cytoplasts include lipids, certain viruses, high temperature, calcium at high pH, or phospholipase C (Gordon (1975) J. Cell Biol. 67:257-280). Optionally, the entire cell containing the nucleus with the optimized genome may be fused with the enucleated recipient cytoplast using, for example, polyethylene glycol as described herein.

Optimized chromosomes in a germ line cell such as a spermatogonial stem cell may be transplanted, for example, into an irradiated testes to complete spermatogenesis in vivo (Brinster (2002) Science 296:2174-2176; Hill & Dobrinski (2006) Reprod. Fertil. Dev. 18:13-18). For example, spermatogonial stem cells may be isolated from bovine testes, cultured in vitro and subsequently injected into testes, resulting in complete regeneration of spermatogenesis (Izadyar et al. (2003) Reprod. 126:765-774). Similar experiments may be done with pig, human and non-human primate spermatogonial cells (Honaramooz et al. (2002) Biol. Reprod. 66:21-28; Tesarik et al. (1999) Lancet 353:555-556; Schlatt et al. (2002) Hum. Reprod. 17:55-62). The optimized chromosomes may be diploid but isogenic to maintain selection through the cross-over events associated with meiosis.

A selected chromosome or chromosomes may be incorporated into an ovarian follicle cell or cells, and the cell or cells differentiated as described herein. Optionally, primary follicle cells containing a selected chromosome or chromosomes may be transplanted into a recipient ovary for further maturation (see, e.g., Carroll et al. (1993) Hum. Reprod. 8:1163-1167).

### EXAMPLE 22

### Isolating Mitochondria for Selection

Mammalian mitochondrial DNA (mtDNA) is a small circular double-stranded DNA approximately 16-17 kb in length, depending upon the species, and contains 37 genes essential for normal mitochondrial function. The mtDNA encodes 2 ribosomal RNAs and 22 transfer RNAs. A non-coding mitochondrial control region contains the main regulatory sequences required for transcription and replication initiation. The mtDNA also encodes 13 proteins which are involved in oxidative phosphorylation within the mitochondria in co-ordination with a number of nuclear-encoded proteins. Somatic cells contain approximately 2000 to 5000 mtDNA copies where as oocytes contain as many as 100,000 mtDNA copies. Most, if not all, of the mtDNA present in an individual is maternally derived from the mtDNA present in the oocyte at the time of conception. Mutations in mtDNA sequences that affect all copies of mtDNA in an individual are termed homoplasmic whereas those mutations that affect only a subset of the mtDNA copies are termed heteroplasmic. The degree of heterogeneity associated with heteroplasmy may vary between different mitochondria in different cells in the same individual.

The mtDNA and/or mitochondria used for selection of optimized mitochondria may be derived from somatic or germ line cells. Examples of somatic cells used for mtDNA analysis may include lymphocytes, oral mucosa or buccal cells, and skeletal muscle (Sgarbi et al. (2006) Biochem. J. 395:493-500; Nekhaeva et al. (2002) Proc. Natl. Acad. Sci. USA 99:5521-5526; Choo-Kang et al. (2002) Diabetes 51:2317-2320).

Rapidly dividing lymphocytes may be an optimal somatic source of mitochondria as they are less “mutated” than post-mitotic, fully differentiated cells (Choo-Kang et al. (2002) Diabetes 51:2317-2320). As such, lymphocytes are isolated from EDTA-treated donor blood by centrifugation through Ficoll-Hypaque Plus (Amersham Biosciences, Piscataway, N.J.) and incubated for 3 hours at 37° C. in RPMI 1640 medium supplemented with 10% fetal bovine serum (FBS), 50 units/ml penicillin and 50 mg/ml streptomycin. After 3 hours, the non-adherent lymphocytes are removed and further cultured in fresh medium for an additional 48 hours. Mitochondria may be isolated from lymphocytes by cell lysis followed by differential centrifugation (Carpentieri & Sordahl (1980) Cancer Res. 40:221-224). As such, lymphocytes are lysed in 0.25 M sucrose, 5 mM Tri-HCl, 5 mM EGTA, and 0.5% bovine serum albumin using either a tight fitting Teflon pestle attached to a motor rotating at approximately 6000 rpm or a tissue homogenizer (e.g. Tekmar Tissumizer, Tekmar Co. Cincinnati Ohio). Alternatively, cells may be lysed manually using a Dounce homogenizer. The homogenate is centrifuged at 480×g for 10 minutes to remove nuclei and heavier cellular debris. The supernatant is then centrifuged at 10,000×g for 10 minutes to generate a mitochondrial pellet. Alternatively, mitochondria may be isolated from lymphocytes using commercially available mitochondria isolation kits (e.g. Mitochondria Isolation Kit for Cultured Cells, Pierce, Rockford, Ill.).

Mitochondria may also be isolated from mammalian skeletal muscle cells (see, e.g., Rooyakers et al. (1996) Proc. Natl. Acad. Sci. USA 93:15364-15369). A skeletal muscle sample is taken from a donor by dissection or needle biopsy, for example. The tissue is homogenized with a Potter-Elvehjem homogenizer (Wheaton, Millville, N.J.) in sufficient volume of a 0.25 M sucrose, 2 mM EDTA, and 10 mM Tris-HCl (pH 7.4) buffer to generate a 5% homogenate. The homogenate is spun at 600×g and the resulting supernatant further spun at 7000×g to generate a mitochondrial pellet.

Mitochondria for selection may alternatively be isolated from other cellular components in a cell lysate by organelle specific fluorescence staining in combination with fluorescence activated organelle sorting (FAOS; see, e.g., Rajotte et al. (2003) Cytometry Part A 55A:94-101). For this approach, whole cells may be treated with fluorescent agents that selectively stain mitochondria such as, for example, MitoTracker Green or Rhodamine 123 (e.g. Invitrogen, Carlsbad, Calif.; Johnson (1980) Proc. Natl. Acad. Sci. USA 77:990-994). The labeled cells are lysed as described above and the mitochondria sorted and collected based on the fluorescent staining. For example, the mitochondria may be sorted using a FACS vantage SE flow cytometer (Becton Dickerson) with an argon laser (488 nm) tuned to a 100 mW output.

Optionally, mitochondria and/or mitochondrial DNA for screening may be isolated using microdissection of specific tissue and cells (see, e.g., Williams & Moraes (2007) Methods Cell Biol. 80:481-501).

### EXAMPLE 23

### Screening Mitochondrial DNA

The mtDNA mutation rate in humans, for example, is at least 10 times that of nuclear genes. These mutations tend to be random such that any base in the mitochondrial genome, coding or noncoding, may be altered. Because every cell in the body harbors hundreds of mitochondria and consequently thousands of mtDNAs, deleterious mutations may occur at some level in all tissues and in both somatic and germ line cells. However, the phenotypic implications of a mtDNA mutation is dependent upon the tissue and cell type in which the mutation occurs. For example, mtDNA mutations arising in somatic tissue may degrade cellular energy production and cause a disease phenotype, but in the long run the mutation will be lost when the individual dies. In contrast, mutations which arise in the female germ line may be transmitted to the next generation where they may be observed as a new mtDNA polymorphism or as a devastating somatic mtDNA disease. As such, somatic mutations are numerous and it is the quantitative differences in expression of these mutations in specific tissues that affects health. Conversely, germ line mutations are more rare, and it is the qualitative nature of the mutation that is important for the phenotype (Wallace (1994) Proc. Natl. Acad. Sci. USA 91:8739-8746).

A number of maternally inherited mtDNA mutations in humans, for example, are associated with disease, including MELAS (mitochondrial encephalomyopathy, lactic acidosis and stroke-like episodes), MERFF (myoclonus epilepsy with ragged-red fibers), MILS (maternally inherited Leigh's syndrome (subacute necrotizing encephalomyopathy)), NARP (neuropathy, ataxia and retinitis pigmentosa), and PEO (progressive external ophthalmoplegia). In addition, somatic mutations in mtDNA have been linked to various aspects of aging, such as sarcopenia or loss of muscle mass, Parkinson's and Alzheimer's diseases, ischemic heart disease, diabetes, cataracts, and hearing loss, (see, e.g.: Kujoth et al (2007) PLOS Genetics 3:e24; Wallace (1994) Proc. Natl. Acad. Sci. USA 91:8739-8746; Liu et al.(1998) Nucleic Acids Res. 26: 1268-1275; and Anisimov (2005) Genet. Res. Camb. 86:127-138; Ohkuba et al. (2001) Clin. Chem. 47:1641-1648; Taylor & Turnbull (2005) Nat. Genet. 6:389-402; U.S. Pat. No. 5,840,493; U.S. Pat. No. 5,976,798).

The complete mitochondrial DNA sequences of a number of mammalian species including, for example, human, mouse, rat, dog, cat, cow, sheep and horse are available from the National Center for Biotechnology Information (NCBI) databases. In addition, specific information regarding, for example, polymorphisms and mutations of the human mitochondrial genome may be accessed through the MITOMAP database (see e.g. Ruiz-Pesini et al. (2007) Nuc. Acids Res. 35:D823-D828). As such, this information may be used to design strategies and probes for screening and optimizing mammalian mitochondrial DNA. For examples, probes may be designed to delineate point mutations or rearrangements within the mtDNA sequence that have been linked to a specific disease or trait. These probes may be used to screen mtDNA by a number of different techniques, including, for example, protein nucleic acid (PNA) probes, polymerase chain reaction (PCR), genome sequencing, high performance liquid chromatography (HPLC), southern analysis and microarrays.

Mitochondrial DNA may be screened in vivo using specific peptide nucleic acid (PNA) oligomers conjugated to a membrane permeable reagent such as, for example, triphenylphosphonium (see, e.g., Muratovska, et al. (2001) Nuc. Acids Res. 29:1852-1863). A PNA oligomer may be designed with a DNA sequence complimentary to a specific mitochondrial mutation and used to assess whether a specific mitochondria contains mtDNA with that specific mutation. For example, a PNA oligomer (from, for example, Applied Biosystems) with sequence complimentary to the human mtDNA L-chain (np 8339-8349) may be used to detect the “myoclonic epilepsy and ragged red fibers” (MERRF) A8344G point mutation in the mitochondrial tRNA-Lys gene.

A PNA oligomer is conjugated to triphenylphosphonium, for example, by pretreating the PNA with 10 mM HEPES, 1 mM EDTA, and 250 nM 2-mercaptoethanol for 1 hour at 40° C. followed by incubation with iodobutyltriphenylphosphonium in HEPES/EDTA/ethanol for an additional 4 hours. The conjugated PNA is purified using reverse phase HPLC. The PNA may be further conjugated to various fluorescent dyes such as FITC, TRITC, and/or BODIPY® derivatives, for example, and/or quantum dots (see, e.g., Dahan (2006) Histochem. Cell Biol. 125:451-456).

Alternatively, PNA access into the mitochondria may be facilitated by conjugation of the PNA to a nuclear encoded protein that normally traverses the mitochondrial membrane such as, for example, cytochrome C oxidase subunit VIII (see, e.g., Chinnery, et al. (1999) Gene Therapy 6:1919-1928). It is anticipated that the fluorescence associated with these probes may be used to isolate the selected mitochondria from whole cell lysates by methods that may include, for example, fluorescence activated organelle sorting and optical tweezers.

Mitochondrial DNA may be screened for various mutations by complete sequence analysis of the mitochondrial genome. For example, 28 to 30 overlapping polymerase chain reaction (PCR) primers evenly spaced along the template may be designed and used to enable sequence analysis across the entire 16-17 kb mtDNA genome. The mtDNA template for PCR may be generated from lymphocytes or a post-mitotic differentiated cell such as skeletal muscle, as described herein. The various PCR fragments are sequenced using, for example, an ABI Prism 377 DNA sequencer (Applied Biosystems, Foster City, Calif.). The resulting sequence may be compared pair-wise, for example, using the BLAST2 sequence alignment tool and reference or wild-type sequence information contained in the MITOMAP database, as described herein. This approach may be used, for example, to find and define mutations associated with mitochondrial disease and maternally inherited diabetes (Choo-Kang et al. (2002) Diabetes 51:2317-2320).

Mutations in mammalian mitochondrial DNA may be assessed using PCR (see, e.g., Naini & Shanske (2007) Methods Cell Biol. 80:437-463; Wong et al. (2002) Clin. Chem. 48:1901-1912). For example, a single base pair substitution at base pair 2,232 of the Japanese Black cattle mtDNA is a strong candidate for mitochondrial effects on meat quality (Mannen et al. (2003) J. Anim. Sci. 81:68-73). At present, more than 200 disease-related mtDNA point mutations located throughout the human genome, for example, have been reported in the MitoMap database (see e.g. Ruiz-Pesini et al. (2007) Nuc. Acids Res. 35:D823-D828; http://www.mitomap.org).

These mutations may be divided into those affecting the mitochondrial tRNA and rRNA genes and mitochondrial protein synthesis, and those affecting protein-encoding genes associated with the respiratory complexes. For example, an A-to-G transition at nt-3243 (A3243G) is the most frequently encountered human mtDNA point mutation and is associated with a spectrum of clinical presentations including MELAS syndrome (mitochondrial encephalomyopathy, lactic acidosis, and stroke-like episodes) and maternally inherited PEO (progressive external ophthalmoplegia).

As such, known mutations in mtDNA may be screened by amplifying an appropriate fragment of mtDNA by PCR, digesting the fragment with a diagnostic restriction enzyme, and analyzing the resulting fragments on a gel, for example. The PCR primers are designed to allow amplification of mtDNA encompassing the potential mutation site (see, e.g., Naini & Shanske (2007) Methods Cell Biol. 80:437-463). Amplification with the PCR primers is completed in the presence of one or more nucleotides tagged with radioactivity, digoxigenin or biotin, for example. The restriction enzymes are chosen such that the PCR fragment cleavage pattern of normal and mutant mtDNA may be differentiated, resulting in different banding patterns on a 12% acrylamide gel, for example.

Point mutations in mtDNA may also be screened using a combination of rolling circle amplification and padlock-based FISH (see, e.g., Tanke et al. (2005) Curr. Opin. Biotechnol. 16:49-54). In this instance, a linear oligonucleotide or padlock probe is designed with 5-prime and 3-prime sequence that is complementary to the target sequence. A linker of irrelevant sequence separates the two ends and carries immunologically detectable residues such as, for example, biotin and/or digoxigenin. Following hybridization of the padlock probe to its target, a ligase reaction covalently links the 3-prime and 5-prime ends of the probe, locking it onto its target strand by circularization which can then be amplified by in situ rolling circle amplification. If the padlock hybridizes to a single base mismatch such as a point mutation or polymorphism, for example, the ligation event will not occur.

Alternatively, quantitative real-time PCR (qPCR) may be used to identify and/or quantify mtDNA point mutations (see, e.g., Naini & Shanske (2007) Methods Cell Biol. 80:437-463). A prerequisite of efficient DNA amplification is the degree of alignment between the 3-prime terminus of a PCR primer and its template. As such, PCR primers may be designed that vary only by a single nucleotide in the region of interest. For example, two primer sets may be designed: one for normal mtDNA and one for mutant mtDNA. One primer may be common to both sets, while the second primer may vary by only a single nucleotide at its 3-prime end. Isolated DNA is amplified with the primer sets using 20-30 cycles of QPCR. During each amplification cycle, the amplified material will increase and may be measured by the fluorescence intensity of a reporter dye. The amount of amplified material is dependent upon the initial concentration of the template. As such, the presence of a mutation may be detected as well as quantified, providing a potential measure of heteroplasmy. A similar approach may be used to assess depletion of mtDNA concentration. The quantity of mtDNA in oocytes, for example, is linked to embryonic developmental competence (see, e.g., Tamassia et al. (2004) Biol. Reprod. 71:697-704).

Mitochondrial DNA mutations may be detected using PCR in combination with high-performance liquid chromatography (see, e.g. Liu et al. (2002) World J. Gastroenterol. 8:426-430; Bayat et al. (2005) Int. J. Immunogenet. 32:199-205). For example, total DNA may be extracted from a blood sample using a commercially available DNA extraction kit (from, for example, Qiagen, Valencia, Calif.) and used as a template for PCR with primers designed to generate specific fragments encompassing various genes in the mtDNA. The PCR fragments are digested with a subset of restriction enzymes to create smaller fragments. The digested fragments are separated using high-performance liquid chromatography (HPLC). The size as well as the HPLC separation characteristics of these fragments may vary depending upon the presence or absence of a mutation.

HPLC combined with PCR may also be used to assess heteroplasmy of a given mtDNA sequence (van den Bosch et al. (2000) Nucleic Acids Res. 28:e89). For example, an A3302G substitution in the tRNA-Leu gene associated with limb-girdle-type myopathy may be detected and quantified using this approach. As such, PCR primers are designed to generate, for example, a 195 base pair fragment containing the potential mutation site. PCR is carried out for 30-40 cycles of, for example, 94° C. for 1 minute, 53° C. for 1 minute, 72° C. for 45 seconds and a final cycle at 72° C. for 7 minutes. The resulting PCR products are separated on HPLC using, for example, a stationary phase DNA Sep® column and a mobile phase of triethylammonium acetate (TEAA) and TEAA with 25% acetonitrile. An acetonitrile gradient may be used to elute the fragments. The resulting chromatographic profile may be compared with that of a PCR fragment from a wild-type or normal mtDNA genome.

Mitochondrial DNA may be screened using single nucleotide polymorphisms (SNPs, see, e.g., Erdogan et al. (2001) Nucleic Acids Res. 29:e36). SNPs, defined as two alternative bases at a particular site, are the most frequent type of DNA sequence variation among individuals, occurring every 500 to 1000 bases. These polymorphisms are for the most part benign, causing no change in function. In some instances, specific polymorphisms may confer an advantage such as, for example, milk yield, fat percentage, and energy in Holstein cows (Boettcher et al. (1996) J. Dairy Sci. 79:647-654; U.S. Pat. No. 5,292,639). As such, the methods described herein may be used to identify mtDNA with advantageous polymorphisms.

Large-scale rearrangements or deletions of mtDNA may be screened, for example, using Southern blot hybridization analysis (see, e.g., Naini & Shanske (2007) Methods Cell Biol. 80:437-463). Large-scale deletions of mtDNA are associated with several clinical conditions, including Kearns-Sayre syndrome (KSS), progressive external ophthalmoplegia (PEO), and Pearson syndrome (PS). Although a number of different deletions have been associated with these clinical conditions, any given individual will typically harbor only a single type of deletion, albeit at potentially different levels in different tissue types (heteroplasmy). Some of these deletions arise sporadically, while others are inherited maternally. A deletion in the mtDNA may be detected using Southern blot analysis. As such, total DNA is isolated from skeletal muscle, for example, digested with one or more restriction enzymes such as, for example, PvuII, BamHI, EagI, EcoRI, HindIII, and/or PstI, separated on an agarose gel, transferred to a nitrocellulose or nylon membrane, hybridized with a labeled mtDNA-selective probe, and the resulting banding pattern assessed for abnormalities. Under normal circumstances, for example, a restriction enzyme that cuts at only a single site in the circular mtDNA sequence should generate a fragment of approximately 16.6 kilobases. The presence of smaller, linearized mtDNA fragments may be indicative of a deletion in the mtDNA.

Mitochondrial DNA may be rapidly screened for mutations using microarray technology (see, e.g., Maitra et al. (2004) Genome Res. 14:812-819). For example, the entire 16 kb mitochondrial genome of a mammal may be tiled as small oligonucleotides on a microarray plate. The tiled mtDNA serves as reference sequence and is used to screen PCR amplified sample mtDNA. Alternatively, microarray screening may be done using a commercially manufactured mitochondrial DNA microarray, for example, GeneChip® Human Mitochondrial Resequencing Array 2.0 (Affymetrix, Santa Clara, Calif.).

Alternatively, mitochondrial DNA may be screened indirectly by assessing activity of proteins encoded by mitochondrial DNA. For example, mutations in mitochondrially encoded ATP synthetase 6 (MT-ATP6) are linked to neuropathy, ataxia, and retinitis pigmentosa (NARP). This protein forms one subunit of ATP synthase or complex V, the last step in oxidative phosphorylation. Mutations in the MT-ATP6 alter the function of ATP synthase, reducing the ability of the mitochondria to make ATP. As such, isolated mitochondria may be screened for relative ATP synthase activity using, for example, commercially available screening kits (from, for example, MitoSciences, Eugene, Oreg.).

### EXAMPLE 24

### Cloning and Manipulation of Mitochondrial DNA

Full-length mitochondrial DNA may be cloned and manipulated using standard molecular biology techniques. For example, full-length circular mitochondrial DNA may be transfected into and manipulated in Escherichia coli bacteria (see, e.g. Yoon & Koob (2003) Nucleic Acids Res. 31:1407-1415; U.S. Patent Application 2007/0128726 A1). Mitochondrial DNA may be selectively isolated from DNase treated mammalian mitochondria. As such, mitochondria are isolated from a mammalian source, as described herein. The isolated mitochondria are incubated in 4000 Kunitz units of DNase I at 37° C. for 1 hour to eliminate nuclear DNA. The treated mitochondria are subsequently washed through a series of sucrose gradients to remove the residual DNase I activity. Mitochondrial DNA is extracted from the mitochondria by digesting the mitochondrial membrane for 3 hours at 50° C. with 10 mg/ml proteinase K in 100 mM NaCl, 10 mM EDTA, 50 mM Tris-HCl, pH 7.4 and 1% SDS. The mtDNA is further extracted with phenol and chloroform and precipitated with ethanol. The mtDNA may be further manipulated by insertion of one or more γ-ori sites using Tn5 transposase in the presence of a PCR derived transposon containing the γ-ori sequence. The mtDNA is inserted into DH5a E. coli using standard electroporation methods and further optimized as needed.

Alternatively, full-length mitochondrial DNA may be first cloned into a Bacillus subtilis genome (BGM) vector using homologous recombination and transfected into E. coli for further manipulation (Yonemura et al. (2007) Gene 391:171-177). As such, two mtDNA segments of 1-2 kb flanking the bulk of the mtDNA are cloned into an E. coil plasmid and integrated into the cloning locus of the BGM vector within B. subtilis. The B. subtilis strain is transformed with total mammalian mtDNA. Total mtDNA may be extracted from isolated mitochondria, as described herein. Alternatively, mtDNA may be extracted from a whole cell lysate using a commercially available kit (see, e.g., mtDNA Extractor CT kit, Wako, Osaka Japan). The mammalian mtDNA sequence is integrated into the BGM vector at the two flanking sites via homologous recombination. As such, the cloned mtDNA becomes integrated into and replicated as part of the B. subtilis genome. Using BReT (Bacillus recombinational transfer), the portion of the B. subtilis genome containing the full-length mtDNA may be extracted in circular form and transferred to E. coli (Yonemura et al. (2007) Gene 391:171-177).

Cloned mitochondrial DNA may be modified to optimize the sequence using standard molecular biology techniques. For example, single base pair changes in the primary sequence to “normalize” a disease associated point mutation, for example, may be generated using user-defined oligonucleotides of, for example, 20 bases and a commercially available site-directed mutagenesis kit (e.g. QuikChange® XL, Stratagene, La Jolla, Calif.). Alternatively, the optimized nucleotide sequence for one or more mitochondrial genes may be synthesized de novo using custom commercial services (e.g. Blue Heron Biotechnology, Bothell, Wash.) and reinserted into the mitochondrial genome.

### EXAMPLE 25

### Insertion of Optimized Mitochondrial DNA Into Mitochondria

Optimized mitochondrial DNA may be introduced into isolated mitochondria by a number of methods. For example, DNA may be introduced into mitochondria by electroporation (Collombet et al. (1997) J. Biol. Chem. 272:5342-5347). Mitochondria for electroporation may be isolated from a mammalian liver, for example, by tissue homogenization followed by successive centrifugations at 700×g and 15,000×g in 10 mM KH₂PO₄, 10 mM KCl, 1 mM EDTA, and 10 mM Tris-HCl, pH 7.4. For electroporation, isolated mitochondria are suspended in 0.33 M sucrose, mixed with the optimized mtDNA and pulsed with a Bio-Rad Gene Pulser™ at a capacitance of 25 microfarads, a resistance of 400 ohms, and a range of field strengths varying between 8 and 20 kV/cm.

Alternatively, optimized mitochondrial DNA may be introduced into isolated mitochondria using bacterial conjugation (Yoon & Koop (2005) Nucleic Acids Res. 33:e139). Bacterium-to-bacterium DNA transfer usually occurs via a mating bridge through a conjugation process that is driven entirely by the donor cell. Furthermore, a broad range of cells types may serve as the DNA recipient, including mammalian cells and mitochondria. As such, conjugative-competent bacteria such as E. coli S17-1, for example, containing, for example, the optimized mtDNA may be incubated with isolated mitochondria. Optionally, the optimized mtDNA may also contain an oriT sequence, for example, to facilitate conjugation mobilization. The optimized DNA will be transferred as a single strand, but the endogenous mtDNA replication system may synthesize the second DNA strand from the origins of DNA replication present in the mtDNA genome (Yoon & Koop (2005) Nucleic Acids Res. 33:e139).

Optionally, optimized mitochondrial DNA may be introduced into mitochondria using mitochondriotropic cationic vesicles (see, e.g. Weissig & Torchilin (2000) Curr. Pharm. Biotechnol. 1:325-346; U.S. Pat. No. 6,627,618). For example, dequalinium, a dicationic compound, can self-assemble into vesicles called DQAsomes and form a complex with DNA. The DQAsomes allow the DNA to interact with the mitochondrial membrane and protects the DNA from nucleases prior to entry into the mitochondria.

Alternatively, optimized mitochondrial DNA may be introduced into mitochondria in intact cells using a mitochondrial leader sequence peptide attached to the mtDNA molecule to facilitate uptake of the mtDNA molecule into the mitochondria (U.S. Patent Application 2004/0192627 A1). For example, the mtDNA may be linked to the pre-sequence peptide of the nuclear encoded cytochrome c oxidase (COX) subunit VIII which is recognized by the mitochondrial protein import machinery. The mtDNA may be conjugated to the pre-sequence peptide using pGeneGRIP™-PNA dependent chemistry (from, for example, Genlantis, San Diego, Calif.). As such, a PNA is designed that hybridizes to a portion of the mtDNA. In addition, the PNA is conjugated, for example, to the pre-sequence peptide of COX subunit VIII. In this manner, the pre-sequence peptide may be linked to the mtDNA.

Optimized mitochondrial DNA may also be incorporated into mitochondria using a physical method, such as, for example, biolistics particle bombardment (Klein et al. (1992) Bio/Technology 10:286-291). For example, optimized mtDNA may be precipitated onto gold particles and the particles accelerated into mitochondria by gun-powder, electric-discharge, or gas-power, for example (see, e.g., Helios Gene Gun, BioRad Laboratories, Inc. Hercules, Calif.).

Optimized mitochondrial DNA may be introduced into mitochondria with existing endogenous mtDNA, creating a heteroplasmic state. Alternatively, optimized mitochondria may be introduced into mitochondria lacking endogenous mtDNA. Mammalian cells lacking mitochondrial DNA, termed ρ⁰ cells, may be generated by sustained treatment of cells with ethidium bromide at a concentration that blocks mtDNA replication with only minimal effect on nuclear DNA (see, e.g., U.S. Pat. No. 5,888,498; Yoon & Koob (2003) Nucleic Acids Res. 31:1407-1415). For example, a mammalian cell line such as LL/2 cells, for example, are exposed to 5 μg/ml ethidium bromide for 4 weeks in high-glucose medium supplemented with 50 μg/ml uridine and 0.1 mg/ml sodium pyruvate. At the end of 4 weeks, clonal ρ⁰ cells are isolated by clonal dilution of the ethidium bromide treated cells. PCR with mtDNA specific primers is used to verify the ρ⁰ state of a clonal population.

Alternatively, ρ⁰ cells may be generated using other mitochondrial toxins such as, for example, ditercalinium, rhodamine 6G, dideoxycytidine, streptozotocin (Inoue et al. (1997) J. Biol. Chem. 272:15510-15515; Bacman & Moraes (2007) Methods Cell Biol. 80:503-524). For example, cells may be treated with 0.05 to 1 μg/ml ditercalinium, an antitumor bis-intercalating agent, for 1 to 4 months, with frequent medium replacement every two days. As above, limiting dilution is used to isolate a clonal population of ρ⁰ cells.

### EXAMPLE 26

### Inserting Selected Mitochondria Into Cells

Mitochondria with optimized mitochondrial DNA may be inserted into a proliferating cell, for example, for propagation of the desired mitochondrial genome for either immediate use, long term culture, or freezing for future use. Mitochondria with optimized mitochondrial DNA generated either by screening mtDNA or by cloning mtDNA, as described herein, may be inserted into a cell with existing endogenous mtDNA, creating a heteroplasmic state. Alternatively, mitochondria with optimized mitochondrial DNA may be inserted into ρ⁰ cells lacking endogenous mtDNA. Methods for generating ρ⁰ cells with toxins and/or reverse transcriptase inhibitors have been described herein. Optionally, ρ⁰ cells may be generated by silencing the endogenous mtDNA polymerase (POL-γ). For example, RNA interference may be used to completely knock-out POL-γ activity, resulting in the loss of detectable mtDNA within 72 hours (Khan & Bennett (2004) J. Bioenerg. Biomembr. 36:387-393). This method may be useful for generating ρ⁰ cells from non-proliferating cells such as neurons, for example, and for generating mtDNA-less ρ⁰ non-proliferating germ cells such as, for example, sperm, spermatids and oocytes.

Alternatively, endogenous mitochondria may be selectively ablated from live cells using femtosecond laser nanoscissors (see, e.g., Shen et al. (2005) MCB 2:17-25). For example, a sample cell may be irradiated with a femtosecond Ti:sapphire laser that delivers 100-fs pulses at 800 nm with a pulse energy of 2-5 nJ at a 1 kHz repetition rate. By tightly focusing these low-repetition, low-energy pulses beneath the cell membrane, is it possible to target organelles, such as, for example, mitochondria through nonlinear processes. Ablation of endogenous mitochondria may be monitored using fluorescence microscopy, for example, with a mitochondrial-selective stain such as MitoTracker Green (from, for example, Molecular Probes, Eugene, Oreg.). Alternatively, mitochondria may be eliminated using microdissection or ablation using microneedles, focused ultraviolet light microscissors, chromophore-assisted laser inactivation, or nanosecond and picosecond lasers (see, e.g., Shen et al. (2005) MCB 2:17-25).

Mitochondria from one cell may be transferred to another cell using a variety of methods. For example, mitochondria from one cell may be incorporated into another by using nuclear transfer techniques in which the nucleus of one cell is transferred into the cytoplasm of an enucleated cell (Bacman & Moraes (2007) Methods Cell Biol. 80:503-524; Meirelles et al. (2001) Genetics 158:351-356; Poulton et al. (2006) Lancet 368:841). Cultured cells may be enucleated using cytochalasin B. To enucleate adherent cells, for example, the cells are treated with 10 μg/ml cytochalasin B in standard culture medium. The culture dish containing the cytochalasin treated cells is flipped upside down and centrifuged at 8000×g for 25 minutes at 35° C., conditions under which the nuclei pop out of the cells and leave cytoplasts attached to the dish. Cells may also be chemically enucleated using 0.5 to 5 μg/ml actinomycin D which is toxic to nuclear DNA but leaves mitochondrial DNA intact (Bayona-Bafaluy et al. (2003) Nucleic Acids Res. 31:e98).

Mitochondria may be transferred from one cell to another using the process of cellular fusion in which a cell containing mitochondrial DNA is fused with a cell lacking mitochondrial DNA such as, for example, ρ⁰ cells (see, e.g. (Bacman & Moraes (2007) Methods Cell Biol. 80:503-524; Kagawa & Hayashi (1997) Gene Ther. 4:6-10; Pye et al. (2006) Nucleic Acids Res. 34:e95). Fusion may be accomplished by incubating the two cell populations in the presence of polyethylene glycol (PEG). For example, ρ⁰ cells are added to enucleated cytoplasts, and cultured for several hours in growth medium, allowing cell-cell contacts to be made. The medium is completely removed, and PEG 1450 is added to the cells for a brief 30 to 60 second period at which time the PEG is removed and the cells washed. The cells are cultured under selection conditions such that only the fused cells survive.

Platelets, which lack nuclear DNA may also serve as a mitochondrial donor (Bacman & Moraes (2007) Methods Cell Biol. 80:503-524). For example, platelets may be isolated from whole blood using a series of centrifugations. Red and white blood cells are pelleted from blood by centrifugation at 150×g for 15 minutes. The platelet-rich plasma is further centrifuged for 35 minutes at 2500×g to pellet the platelets. The platelet mtDNA of one or more individuals may be screened using the methods described herein. The platelets containing the optimized mtDNA may then be fused with ρ⁰ cells or other nuclear donor, as described herein.

### EXAMPLE 27

### Generating Germ Line Cells with Selected Mitochondria

Optimized mitochondria derived using the methods described herein may be incorporated into germ line cells to produce progeny with the optimized mitochondria. In general, mitochondrial DNA is inherited maternally. Human oocytes, for example, have 200,000 mtDNA copies per oocyte while sperm may have as little as 10 mtDNA copies per cell (May-Panloup et al. (2003) Hum. Reprod. 18:550-556).

Selected mitochondria may be microinjected into pronucleus stage embryos (Shitara et al. (2000) Genetics 156:1277-1284). Selected mitochondria stored in a proliferating cell line, for example, may be isolated using the procedures described herein and injected into embryos at the pronucleus stage using, for example, a Piezo micromanipulator with 1-2 picoliters of mitochondrial suspension. Alternatively, enucleated cyoplasts from a proliferating cell line containing the selected mitochondria may be injected into an oocyte (Takeda et al. (2005) Biol. Reprod. 72:1397-1404). A proliferating cell line may include, for example, a somatic cell, a stem cell, an embryonic stem cell, or a primordial germ cell. For example, mitochondria and associated mtDNA isolated from somatic cells and injected into pronucleus stage embryos are able to persist during embryogenesis and are detected in the cells of progeny (Shitara et al. (2000) Genetics 156:1277-1284).

Alternatively, selected mitochondria may be introduced into the female germ line by the transfer of cytoplasm from one oocyte into another (see, e.g., Barritt et al. (2001) Hum. Reprod. Update 7:428-435; Levy et al. (2004) Hum. Reprod. Update 10:241-250; van Blerkom et al. (1998) Hum. Reprod. 13:2857-2868; Food & Drug Administration: BRMAC Briefing Document 2002). In one instance, mitochondria with selected mtDNA may be microinjected into the oocyte, for example, in the presence of endogenous mtDNA.

Alternatively, endogenous mtDNA in the oocyte may be first eliminated using the chemical or physical methods described herein. Optionally, the endogenous cytoplasm may be removed by aspiration. Methods have been described for the aspiration of metaphase II chromosomes from an oocyte (see, e.g., Wakayama et al. (1998) Nature 394:369-374; Wakayama (2007) J. Reprod. Develop. 53:13-26). As such, the cytoplasm may also be removed from an oocyte via aspiration. The oocyte may be flushed with a biologically compatible buffer such as, for example, physiologically buffered saline. The oocyte cytoplasm may be replaced with selected mitochondria. In some instances, the aspirated oocyte cytoplasm may be centrifuged to remove endogenous mitochondria and reinjected into the oocyte along with the selected mitochondria.

Alternatively, selected mitochondria may be incorporated into embryonic stem cells or primordial germ cells prior to differentiation into oocytes. Embryonic stem cells, for example, are derived from mammalian preimplantation blastocysts and have the ability to self-renew indefinitely and to differentiate into a wide range of cell types, including oocytes (Hubner et al. (2003) Science 300:1251-1256; West et al. (2006) Nature Protocols 1:2026-2036). As such, endogenous mitochondria in the embryonic stem cells may be ablated using toxins, physical ablation, and/or RNA interference as described herein. Selected mitochondria are introduced into the embryonic cells lacking endogenous mitochondria using the methods described herein.

The modified embryonic stem cells may be frozen for use in the future, propagated in the absence of differentiation agents to increase the number of cells with the selected mitochondria, or allowed to progress down the path towards oocyte differentiation. In the latter instance, embryonic stem cells from mice, for example, are grown in ES medium with heat-inactivated serum in the absence of feeder cells or the growth factors required for self-renewal. As such, the cells proceed down various differentiation paths. At day 7, cells may be sorted based on expression levels of Oct4, a germ line specific gene and further assessed for expression of other germ line markers, including, for example c-kit, Vasa, synaptonemal complex protein 3 (SCP3) and meiosis-specific homologous recombination gene (DMC1) (Hubner et al. (2003) Science 300:1251-1256). After further culturing for 16 to 20 days, follicle-like structures may begin to form and as early 26 days of culture, oocyte-like cells may become apparent. The embryonic stem cell-derived oocytes may be capable of fertilization under the appropriate conditions. Alternatively, selected mitochondria that have been harbored in embryonic stem cell-derived oocytes may be isolated from these cells and subsequently introduced into a naturally derived oocyte.

### EXAMPLE 28

### Compatibility of mtDNA and Nuclear DNA

In some instances, it may be beneficial to determine the compatibility of the selected mtDNA with the nuclear DNA of the recipient cell. While the mtDNA encodes a number of protein subunits comprising the mitochondrial respiratory chain complex involved in oxidative phosphorylation, the bulk of the proteins involved in this process are encoded in the nuclear DNA. In addition, there are a number of nuclear-encoded regulatory factors that are required for normal mtDNA transcription and replication, including TFAM, TFB1M (or TFB2M), MtRNAPol, PolG, MtSSB, MTERF, and RNase MRP. Failure to co-ordinate transcription between nuclear-derived factors and mtDNA may have serious implications for a functional mitochondrial respiratory chain (Spikings et al. (2006) Hum. Reprod. Update 12:401-415).

For mitochondrial function, the various subunits and factors encoded by the mtDNA and the nuclear DNA work together. For example, the transfer of mtDNA from the rat, Rattus norvegicus into mtDNA-less ρ⁰ cells from the mouse Mus spretus results in chimera cybrids in which the mitochondria exhibit very low mitochondrial respiratory activity, even though the rat mtDNA replicates and induces normal translation of rat mtDNA-encoded polypeptides (Yamaoka et al. (2000) Genetics 155:301-307). Similarly, the transfer of mtDNA from orangutan, lemurs and species representative of Old- and New-World monkeys into human ρ⁰ cells results in little or no mitochondrial respiratory activity (Kenyon & Moraes (1997) Proc. Natl. Acad. Sci. USA 94:9131-9135).

In contract, normal oxidative phosphorylation is restored to varying degrees in human ρ⁰ cells when combined with mtDNA from common chimpanzee, pigmy chimpanzee, and gorilla, suggesting that transfer of mtDNA between genetically very similar species results in functional mitochondria. In general, intraspecies mtDNA transfer appears to be less problematic. However, certain polymorphisms within the nuclear DNA and mtDNA of a given species encoding, for example, respiratory chain subunits and/or mitochondrial regulatory factors may confer more or less mitochondrial function. As such, the screening methods described herein may be used to isolate a nuclear chromosome or chromosomes which will co-ordinate with either endogenous or selected mitochondrial DNA.

Mitochondrial function may be assessed using biochemical assays which measure the activity of the various complexes forming the mitochondrial respiratory chain complex such as, for example, Complex I, Complex II/III, Complex IV as well as mitochondrial citrate synthase and ATP synthase. For example, complex IV (cytochrome c oxidase) activity may be measured using a 15 minute, 2-point rate assay assessing the oxidation of cytochrome c at a first wavelength of 546 nm and a secondary wavelength of 570 (Kramer et al. (2005) Clin. Chem. 51:2110-2116). A cell homogenate is incubated for 5 min with 40 mmol/L potassium phosphate (pH 7.2) prior to addition of reduced cytochrome c at a final concentration of 0.015 mmol/L. The resulting decrease in absorbance is measured for 5 minutes. K₃Fe(CN)₆ is added to a final concentration of 0.015 mmol/L, and the absorbance measured for an additional 5 minutes. Mitochondrial activity assays may be developed for high throughput automation (see, e.g., Kramer et al. (2005) Clin. Chem. 51:2110-2116). Alternatively, assay kits measuring the activity of, for example, complexes IV and V, may be obtained from a commercial source (see, e.g., MitoSciences, Eugene, Oreg.). As such, mitochondrial activity assays may be used to assess the function of mitochondria before and/or following transfer to a recipient cell.

Optionally, mitochondrial function may be measured by assessing oxygen consumption, a sensitive index of respiratory function. For example, the rate of oxygen consumption may be measured by trypsinizing cells, incubating the cell suspension in phosphate-buffered saline, and recording oxygen consumption in a polarographic cell (1.0 ml) at 37° C. with a Clark-type oxygen electrode (see, e.g., Yamaoka et al. (2000) Genetics 155:301-307).

### EXAMPLE 29

### Selecting Female Haploid Genome

A chromosome, chromosomes or genome may be selected from a diversified female haploid genome using spermatogenesis. As such, the genome from a female mammal may be transferred into a sperm progenitor cell. The latter is allowed to proliferate and differentiate through meiosis and the associated cross-over events. Alternatively, a female stem cell, embryonic stem cell or primordial germ cell may be induced to differentiate down the spermatogenic path. As such, it becomes possible to take advantage of the varied recombination events possible during sperm maturation. A chromosome or chromosomes in the resulting sperm, spermatids or sperm-like cells are screened to find the best or desired combination of chromosomes using the methods described herein. The cell or cells containing the best or desired combination of chromosomes may be used for fertilization of the donor's own oocyte or oocytes. Alternatively, the selected cell or cells may be used for fertilization of another individual's oocyte or oocytes. Optionally, the best or desired combination of chromosomes may be isolated from the selected cell or cells and put back into a female progenitor germ cell and allowed to differentiate to an oocyte for fertilization by male donor sperm. Alternatively, the best or desired combination of chromosomes may be put into a somatic or embryonic stem cell and the nucleus used for cloning by nuclear transfer.

A female nuclei containing the genome of a female mammal may be isolated from a diploid germ cell, for example, a differentiated primordial germ cell or a primary oocyte. Alternatively, a female nuclei containing the genome of a female mammal may be isolated from a proliferating somatic cell, such as for example, a lymphocyte. Alternatively, a female nuclei containing the genome of a female mammal may be isolated from a non-proliferating somatic cell, such as for example, a cumulus cell.

The nucleus of a female mammalian donor may be transferred into a proliferating male germ line cell. As such, the female nucleus may be transferred into a spermatogonial cell, a primordial germ line cell or an embryonic stem cell. The recipient cell may be enucleated using cytochalasin B in combination with centrifugation using the methods described herein. A female nucleus may be transferred to the cytoplast using microinjection techniques. Alternatively, karyoplasts of the female nuclei may be formed using the methods described here in and used for fusion using for example polyethylene glycol, lipids, certain viruses, high temperature, calcium at high pH, or phospholipase C (Gordon (1975) J. Cell Biol. 67:257-280). Optionally, the entire cell containing the female nucleus may be fused with the enucleated recipient cytoplast using, for example, polyethylene glycol as described herein. The resulting recipient cells are induced to enter spermatogenesis through to meiosis and the cross-over events.

Optionally, a male Y chromosome may be substituted for one of the two female X chromosomes in the female genome (see, e.g., U.S. Patent Application 2002/0174449 A1). As such, one of the endogenous X chromosomes in the female nucleus may be ablated using, for example, laser ablation as described herein. A Y chromosome may be isolated from a male cell and transferred into the female nucleus using microinjection, for example, as described herein.

It is anticipated that sperm and eggs may be derived from the same human embryonic stem cell line, irrespective of whether the somatic cell was derived from a male or a female (Darby (2006) Human Fertilization & Embryology Authority, UK, Scientific and Clinical Advances Group, The use of in vitro derived gametes). As such an embryonic stem cell with a female genome may be differentiated into a sperm. Alternatively, a female genome in a stem cell, an embryonic stem cell or primordial germ line cell may be modified such that one of the X chromosomes is replaced by a Y chromosome. As such, the modified stem cell, embryonic stem cell or primordial germ line cell may be differentiated into a sperm-like cell using the methods described herein.

Spermatogenesis may be induced in vitro using the methods described herein. Alternatively, spermatogenesis may be induced in vivo using transplantation techniques. Spermatogonial stem cells may be transplanted, for example, into an irradiated testes to complete spermatogenesis in vivo (Brinster (2002) Science 296:2174-2176; U.S. Pat. No. 6,316,692). For example, spermatogonial stem cells may be isolated from bovine testes, cultured in vitro and subsequently injected into an irradiated testes, resulting in complete regeneration of spermatogenesis (Izadyar et al. (2003) Reprod. 126:765-774). Similar experiments may be done with human and non-human primate spermatogonial cells (Tesarik et al. (1999) Lancet 353:555-556; Schlatt et al. (2002) Hum. Reprod. 17:55-62).

Alternatively, a xenogeneic system may be used in which spermatogonial stem cells from one species, such as rat, for example, are transplanted into mouse testes for completion of differentiation (Shinohara et al. (2006) Proc. Natl. Acad. Sci. USA 103:13624-13628). As such, spermatogonial stem cells containing the female nuclei may be transplanted into an irradiated testes and allowed to grow for 3 to 4 months, for example, depending upon the cycle time for spermatogenesis in the species in use. At some point, spermatids or sperm which have progressed through second meiosis and are in the haploid state may be harvested, and the chromosomes screened using the methods described herein.

Alternatively, in vivo spermatogenesis may be accomplished using transplantation with primordial germ cells (PGCs; Chuma et al. (2005) Development 132:117-122). As such, PGCs containing a female nucleus may be transplanted into an irradiated mammalian testes. For example, PGCs may be isolated from a mouse at 6 to 16 days post-coitum, a female nucleus added and the resulting cells transplanted into a recipient testes, and grown for 3 to 4 months.

Alternatively, in vivo spermatogenesis may be accomplished by transplanting differentiated embryonic stem cells into testes (Toyooka et al. (2003) Proc. Natl. Acad. Sci. USA 100: 11457-11462). Embryonic stem cells from mouse, for example, may be differentiated into PGCs in the presence of feeder cells producing BMP4 and/or BMP8. At some point during differentiation, the female nucleus with or without a Y chromosome is introduced into the cells. Cell aggregates are transplanted into a recipient testis capsule and after 6 to 8 weeks, round spermatids may be evident.

Sperm, spermatids, or differentiated sperm-like cells containing the female genome and having undergone meiosis and cross-over events are isolated either from in vitro differentiation of cultured cells or from in vivo differentiation in the testes from ejaculates or dissection. As such, the chromosomes are screened using for example a tagged PNA, a polyamide, or an oligonucleotide as described herein. A cell or cells containing the selected combination of chromosomes may be used immediately for insemination or transferred to a female germ line cell.

The sperm, spermatid or differentiated sperm-like cells may be immediately used for insemination of a recipient oocyte. The recipient oocyte may be from the female nucleus donor. Alternatively, the recipient oocyte may be from another female. As such, the selected sperm with the female nucleus may be incubated with a receptive oocyte to induce fertilization. Alternatively, the selected sperm, spermatid or differentiated sperm-like cell may be injected into the receptive oocyte using standard procedures. The resulting embryo may then be transferred to a receptive uterus for further development.

Alternatively, the nucleus from the selected sperm, spermatid or differentiated sperm-like cell containing the female genome may be transferred back into a female germ line cell for feminization, for example. As such, the selected female haploid genome may undergo diploidization as described herein and placed into an embryonic stem cell or a primordial germ line cell and allowed to differentiate.

In one aspect, the disclosure is drawn to one or more methods comprising receiving a first input associated with a first possible dataset, the first possible dataset including data representative of one or more target genetic characteristics, wherein at least one of the one or more target genetic characteristics is a genetic characteristic other than sex chromosome identity; and determining parameters for selecting one or more reproductive components based on the first possible dataset. In some embodiments, the one or more methods comprise receiving a first input associated with a first possible dataset, the first possible dataset including data representative of one or more target genetic characteristics, wherein at least one of the one or more target genetic characteristics is a non-gender-specific genetic characteristic, or is a genetic characteristic other than gender. One or more of these methods may be used as part of one or more methods for selecting one or more germ line genomes at least partially based on one or more genetic characteristics of one or more of the one or more germ line genomes and/or implemented on one or more apparatus 410 for selecting one or more germ line genomes at least partially based on one or more genetic characteristics of one or more of the one or more germ line genomes.

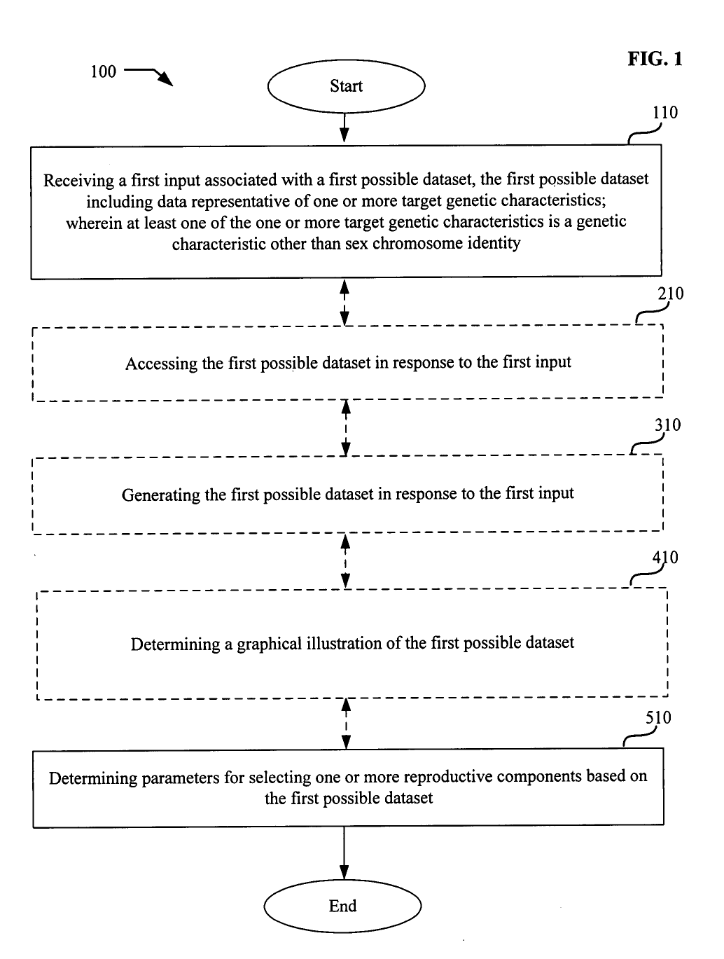

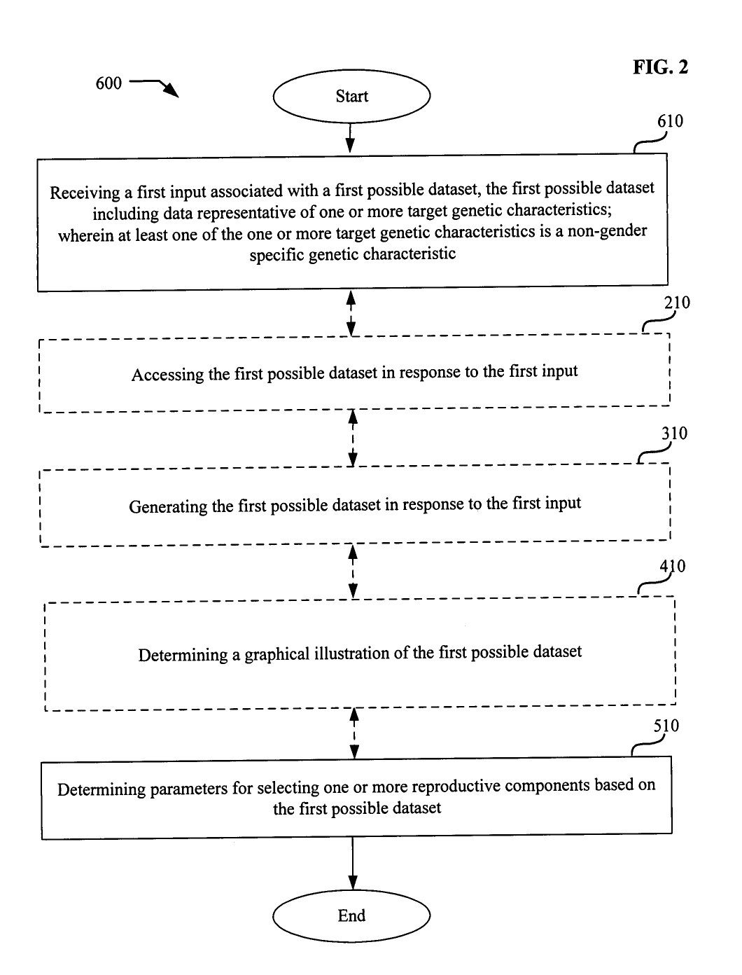

FIG. 1, FIG. 2, and FIG. 3 show operational flow 100, operational flow 600, and operational flow 700, respectively, representing illustrative embodiments of operations related to determining parameters for selecting one or more reproductive components based on the first possible dataset. In FIG. 1, FIG. 2, and FIG. 3, and in the following figures that include various illustrative embodiments of operational flows, discussion and explanation may be provided with respect to apparatus and methods described herein, and/or with respect to other examples and contexts. The operational flows may also be executed in a variety of other contexts and environments, and or in modified versions of those described herein. In addition, although some of the operational flows are presented in sequence, the various operations may be performed in various repetitions, concurrently, and/or in other orders than those that are illustrated.

After a start operation, the operational flow 100 moves to a receiving operation 110, receiving a first input associated with a first possible dataset, the first possible dataset including data representative of one or more target genetic characteristics, wherein at least one of the one or more target genetic characteristics is a genetic characteristic other than sex chromosome identity. After a start operation, the operational flow 600 moves to a receiving operation 610, receiving a first input associated with a first possible dataset, the first possible dataset including data representative of one or more target genetic characteristics, wherein at least one of the one or more target genetic characteristics is a non-gender-specific genetic characteristic. After a start operation, the operational flow 700 moves to a receiving operation 710, receiving a first input associated with a first possible dataset, the first possible dataset including data representative of one or more target genetic characteristics, wherein at least one of the one or more target genetic characteristics is a genetic characteristic other than gender.

The operational flow 100 optionally moves to an accessing operation 210, accessing the first possible dataset in response to the first input. For example, data representative of one or more target genetic characteristics may be accessed.

The operational flow 100 optionally moves to a generating operation 310, generating the first possible dataset in response to the first input. For example, data representative of one or more target genetic characteristics may be generated.

The operational flow 100 optionally moves to a determining operation 410, determining a graphical illustration of the first possible dataset. For example, data representative of one or more target genetic characteristics may be graphically represented.

Then, the operational flow 100 moves to a determining operation 510, determining parameters for selecting one or more reproductive components based on a first possible dataset. For example, one or more parameters may include, but are not limited to one or more target genetic characteristics and/or one or more genetic characteristics of one or more reproductive components.

One or more of operations 110 (and/or 610 and/or 710) through 510 may be performed or repeated, as appropriate under the circumstances, prior to an end operation.

Operations 110 to 510 may be performed with respect to a digital representation (e.g. digital data) of, for example, data representative of one or more target genetic characteristics. The logic may accept a digital or analog (for conversion into digital) representation of an input and/or provide a digitally-encoded representation of a graphical illustration, where the input may be implemented and/or accessed locally or remotely.

Operations 110 to 510 may be performed related to either a local or a remote storage of the digital data, or to another type of transmission of the digital data. In addition to inputting, accessing querying, recalling, calculating, determining or otherwise obtaining the digital data, operations may be performed related to storing, assigning, associating, displaying or otherwise archiving the digital data to a memory, including for example, sending and/or receiving a transmission of the digital data from a remote memory. Accordingly, any such operations may involve elements including at least an operator (e.g. human or computer) directing the operation, a transmitting computer, and/or receiving computer, and should be understood to occur in the United States as long as at least one of these elements resides in the United States.

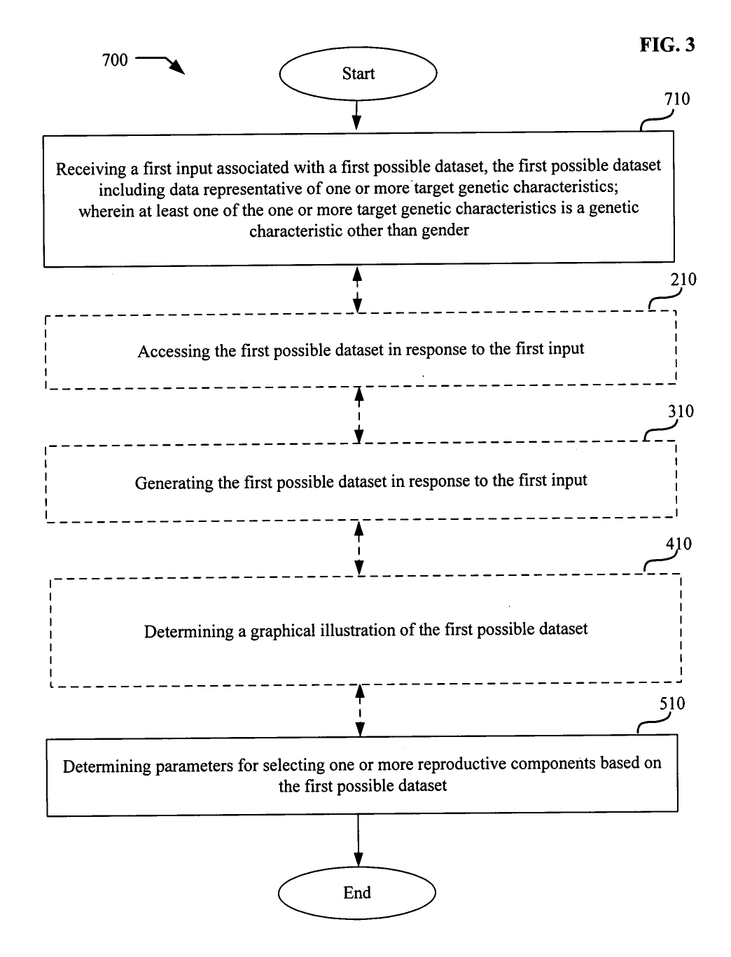

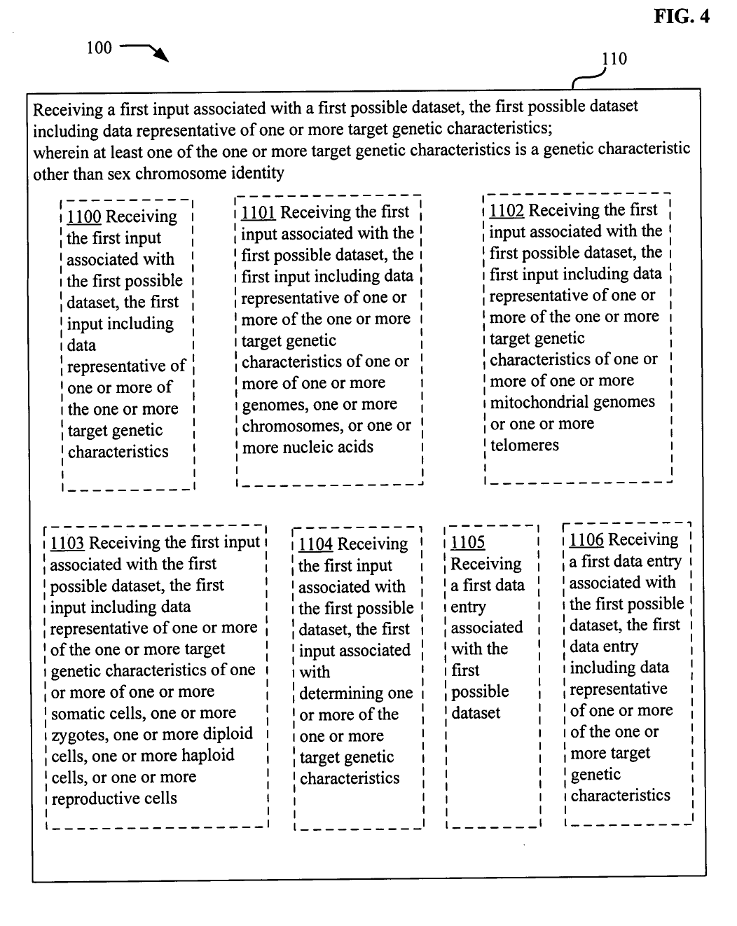

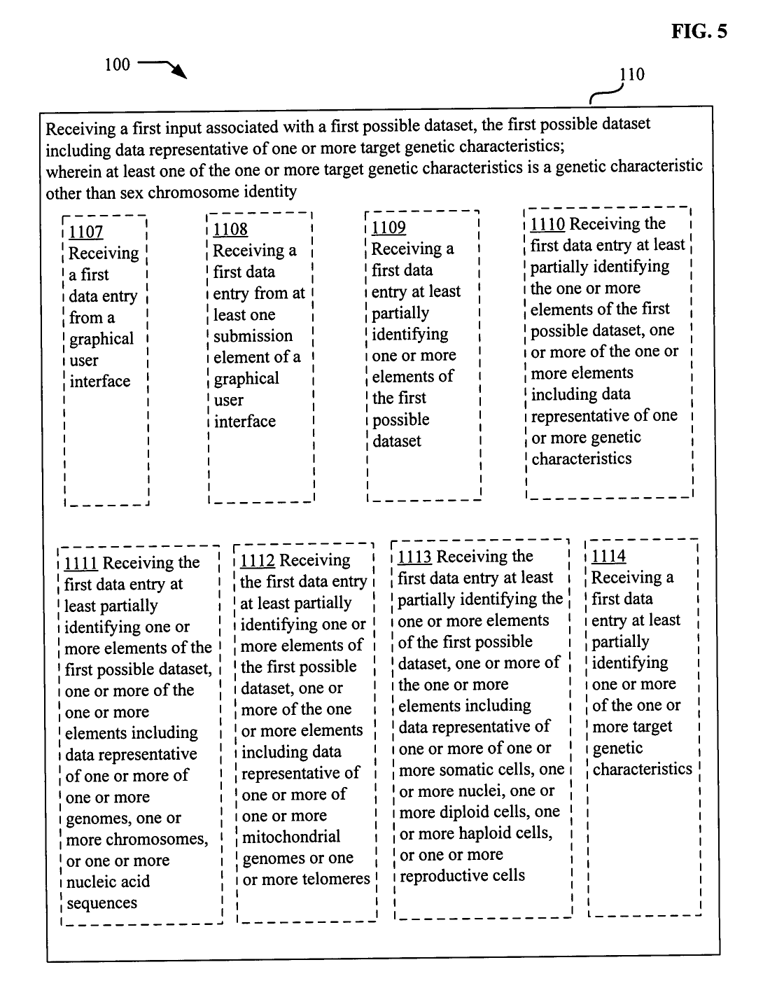

FIG. 4 illustrates optional embodiments of the operational flow 100 of FIG. 1, and analogous embodiments of the operational flow 100 of FIG. 2 and/or FIG. 3 are expressly envisioned. FIG. 4 shows illustrative embodiments of the receiving operation 110, receiving a first input associated with a first possible dataset, the first possible dataset including data representative of one or more target genetic characteristics, wherein at least one or more one or more target genetic characteristics is a genetic characteristic other than sex chromosome identity, including operations receiving types of inputs and data entry and may include at least one additional operation. Receiving operations may optionally include, but are not limited to, operation 1100, operation 1101, operation 1102, operation 1103, operation 1104, operation 1105, operation 1106, operation 1107, operation 1108, operation 1109, operation 1110, operation 1111, operation 1112, operation 1113, and/or operation 1114.

At the optional operation 1100, receiving a first input associated with a first possible dataset comprises receiving the first input associated with the first possible dataset, the first input including data representative of one or more of the one or more target genetic characteristics.

In some embodiments, one or more of the one or more target genetic characteristics are selected from the group consisting of genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements. In some embodiments, one or more of the genetic attributes include one or more of one or more physical attributes, one or more psychological attributes, or one or more mental attributes.

In some embodiments, one or more of the one or more physical attributes are selected from the group consisting of characteristics associated with vision, strength, flexibility, speed, coordination, gait, lactation, fertility, weight, pelt, skin, body type, skeleto-muscular, longevity, and intelligence. In some embodiments, one or more of the one or more physical attributes are selected from the group consisting of characteristics associated with hair, eyes, height, weight, skin, fur, fleece, and wool. In some embodiments, one or more of the one or more physical attributes are selected from the group consisting of characteristics associated with hair pattern, hair color, eye color, eye sight, bone length, bone density, skin color, fur thickness, fur color, fur texture, fleece color, fleece thickness, wool thickness, and wool color. In some embodiments, one or more of the one or more physical attributes include disposition.

At the optional operation 1101, receiving a first input associated with a first possible dataset comprises receiving a first input associated with the first possible dataset, the first input including data representative of one or more of the one or more target genetic characteristics of one or more of one or more genomes, one or more chromosomes, and/or one or more nucleic acids.

At the optional operation 1102, receiving a first input associated with a first possible dataset comprises receiving a first input associated with the first possible dataset, the first input including data representative of one or more of the one or more target genetic characteristics of one or more of one or more mitochondrial genomes, and/or one or more telomeres.

At the optional operation 1103, receiving a first input associated with a first possible dataset comprises receiving a first input associated with the first possible dataset, the first input including data representative of one or more of the one or more target genetic characteristics of one or more of one or more somatic cells, one or more germ line cells, one or more zygotes, one or more diploid cells, one or more haploid cells, and/or one or more reproductive cells. In some embodiments, the first input includes data representative of one or more of the one or more target genetic characteristics of one or more of one or more sperm, one or more spermatids, one or more spermatogonia, one or more primary spermatocytes, or one or more secondary spermatocytes. In some embodiments, the first input includes data representative of one or more genetic characteristics of one or more of one or more ova, one or more first polar bodies, or one or more second polar bodies.

At the optional operation 1104, receiving a first input associated with a first possible dataset comprises receiving a first input associated with the first possible dataset, the first input associated with determining one or more of the one or more target genetic characteristics. In some embodiments, the one or more target genetic characteristics are selected from the group consisting of genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 1105 and/or 1106, receiving a first input associated with a first possible dataset comprises receiving a first data entry associated with the first possible dataset, the first data entry optionally including data representative of one or more of the one or more target genetic characteristics. In some embodiments, the one or more target genetic characteristics selected from the group consisting of genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 1107 and/or 1108, receiving a first input associated with a first possible dataset comprises receiving a first data entry from a graphical user interface, optionally from at least one submission element of a graphical user interface, and optionally at least partially identifying one or more elements of the first possible dataset.

At the optional operation 1109 and/or 1110 and/or 1111 and/or 112 and/or 1113, receiving a first input associated with a first possible dataset comprises receiving a first data entry at least partially identifying one or more elements of the first possible dataset, one or more of the one or more elements optionally including data representative of one or more genetic characteristics. In some embodiments, one or more of the one or more elements including data representative of one or more genetic characteristics selected from the group consisting of single nucleotide polymorphisms, haplotypes, allelic markers, alleles, disease markers, genetic abnormalities, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

In some embodiments, one or more of the one or more elements optionally including data representative of one or more of one or more genomes, one or more chromosomes, and/or one or more nucleic acid sequences. In some embodiments, one or more of the one or more elements optionally including data representative of one or more of one or more mitochondrial genomes and/or one or more telomeres. In some embodiments, one or more of the one or more elements optionally including data representative of one or more of one or more somatic cells, one or more germ line cells, one or more nuclei, one or more diploid cells, one or more haploid cells, or one or more reproductive cells. In some embodiments, one or more of the one or more elements optionally including data representative of one or more of one or more sperm, one or more spermatids, one or more spermatogonia, one or more primary spermatocytes, or one or more secondary spermatocytes. In some embodiments, one or more of the one or more elements optionally including data representative of one or more of one or more ova, one or more first polar bodies, or one or more second polar bodies.

At the optional operation 1114, receiving a first input associated with a first possible dataset comprises receiving a first data entry at least partially identifying one or more of the one or more target genetic characteristics. In some embodiments, one or more of the one or more target genetic characteristics selected from the group consisting of genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

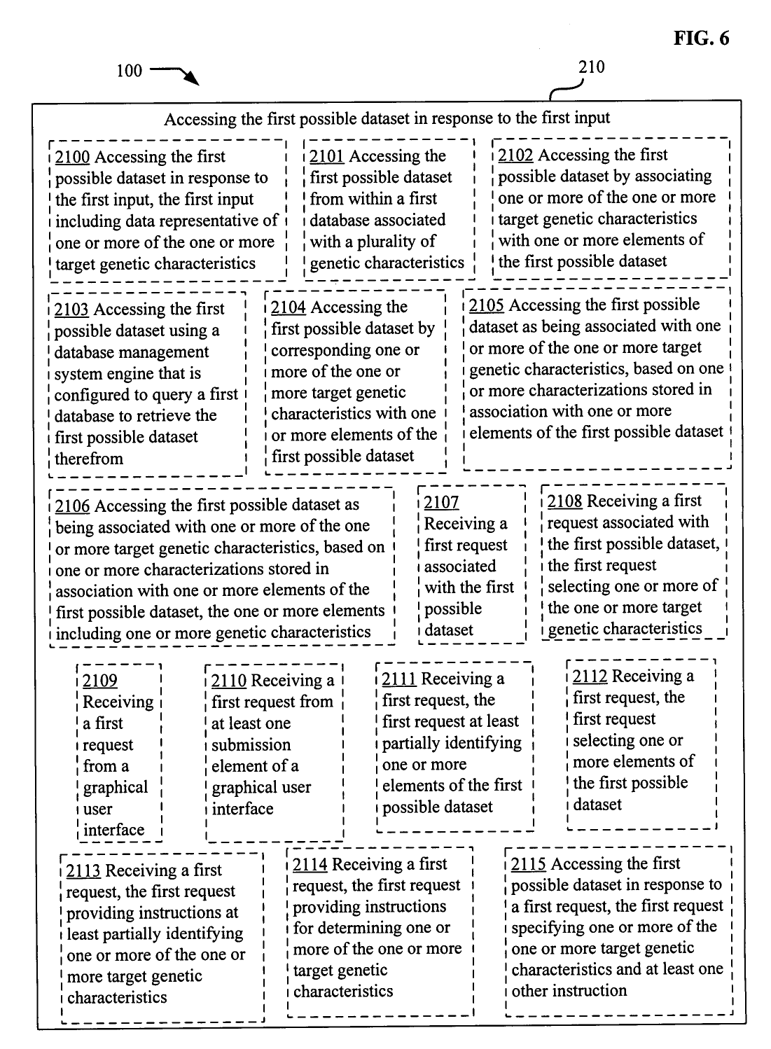

FIG. 6 illustrates optional embodiments of the operational flow 100 of FIG. 1. FIG. 6 shows illustrative embodiments of the optional accessing operation 210, including operations accessing the first possible dataset in response to the first input, and may include at least one additional operation. Accessing operations may optionally include, but are not limited to, operation 2100, operation 2101, operation 2102, operation 2103, operation 2104, operation 2105, operation 2106, operation 2107, operation 2108, operation 2109, operation 2110, operation 2111, operation 2112, operation 2113, operation 2114, and/or operation 2115.

At the optional operation 2100, accessing the first possible dataset in response to the first input comprises accessing the first possible dataset in response to the first input, the first input including data representative of one or more of the one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 2101, accessing the first possible dataset in response to the first input comprises accessing the first possible dataset from within a first database associated with a plurality of genetic characteristics. In some embodiments, one or more of the one or more genetic characteristics selected from the group consisting of single nucleotide polymorphisms, haplotypes, allelic markers, alleles, disease markers, genetic abnormalities, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 2102 and/or operation 2104, accessing the first possible dataset in response to the first input comprises accessing the first possible dataset by associating and/or correlating and/or corresponding data representative of one or more of the one or more target genetic characteristics with one or more elements of the first possible dataset. In some embodiments, one or more of the one or more target genetic characteristics are selected from the group consisting of genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements with the one or more elements of the first possible dataset.

At the optional operation 2103, accessing the first possible dataset in response to the first input comprises accessing the first possible dataset using a database management system engine that is configured to query a first database to retrieve the first possible dataset therefrom.

At the optional operation 2105 and/or 2106, accessing the first possible dataset in response to the first input comprises accessing the first possible dataset as being associated and/or correlated and/or corresponded with data representative of one or more of the one or more target genetic characteristics, based on one or more characterizations stored in association with one or more elements of the first possible dataset, the one or more elements optionally including one or more genetic characteristics.

At the optional operation 2107 and/or 2108, receiving a first input associated with a first possible dataset comprises receiving a first request associated with the first possible dataset, the first request optionally selecting data representative of the one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 2109 and/or 2110 and/or 2111 and/or 2112 and/or 2113, and/or 2114, and/or 2115, receiving a first input associated with a first possible dataset comprises receiving a first request from a graphical user interface, optionally from at least one submission element of a graphical user interface, optionally at least partially identifying one or more elements of the first possible dataset and/or optionally selecting one or more elements of the first possible dataset and/or optionally providing instructions identifying and/or determining and/or specifying one or more of the one or more target genetic characteristics, and optionally providing at least one other instruction.

In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

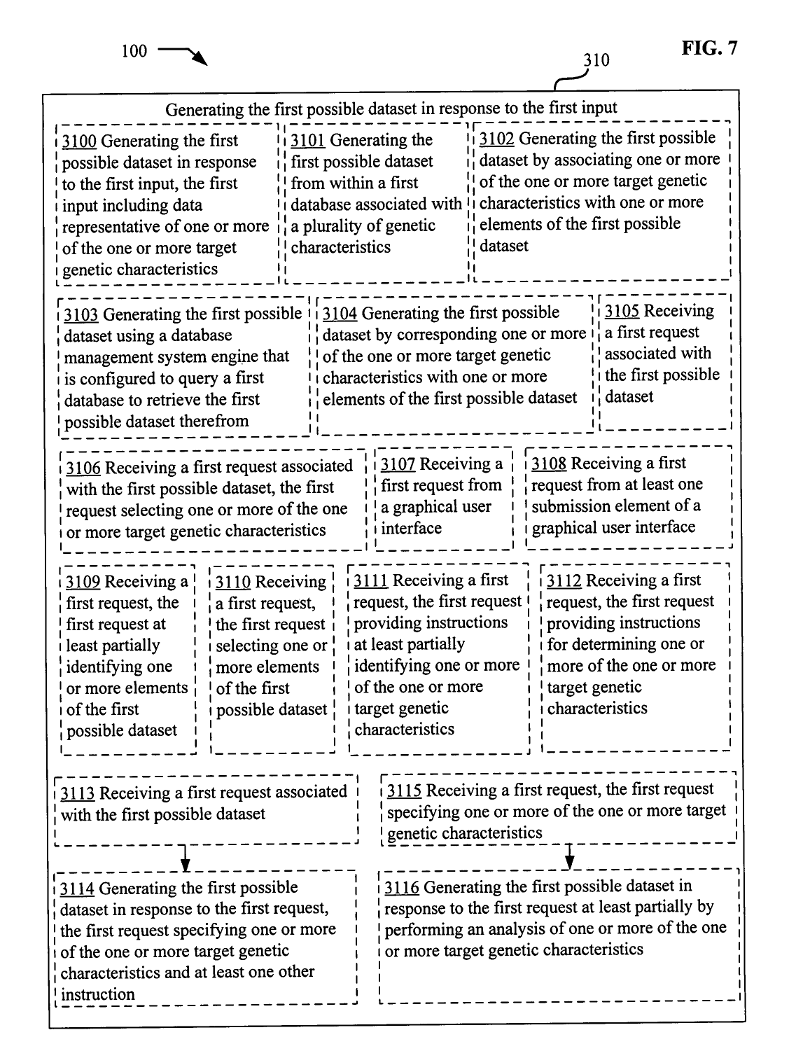

FIG. 7 illustrates optional embodiments of the operational flow 100 of FIG. 1. FIG. 7 shows illustrative embodiments of the optional generating operation 310, including operations generating the first possible dataset in response to the first input, and may include at least one additional operation. Generating operations may optionally include, but are not limited to, operation 3100, operation 3101, operation 3102, operation 3103, operation 3104, operation 3105, operation 3106, operation 3107, operation 3108, operation 3109, operation 3110, operation 3111, operation 3112, operation 3113, operation 3114, operation 3115, and/or operation 3116.

At the optional operation 3100, generating the first possible dataset in response to the first input comprises generating the first possible dataset in response to the first input, the first input including data representative of one or more of one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3101, generating the first possible dataset in response to the first input comprises generating the first possible dataset from within a first database associated with a plurality of genetic characteristics. In some embodiments, one or more of the one or more genetic characteristics are selected from the group consisting of one or more single nucleotide polymorphisms, haplotypes, allelic markers, alleles, disease markers, genetic abnormalities, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3102, generating the first possible dataset in response to the first input comprises generating the first possible dataset by associating data representative of one or more of the one or more target genetic characteristics with one or more elements of the first possible dataset. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3103, generating the first possible dataset in response to the first input comprises generating the first possible dataset using a database management system engine that is configured to query a first database to retrieve the first possible dataset therefrom.

At the optional operation 3104, generating the first possible dataset in response to the first input comprises generating the first possible dataset by corresponding data representative of one or more of the one or more target genetic characteristics with one or more elements of the first possible dataset. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3105 and/or 3106, receiving a first input associated with a first possible dataset comprises receiving a first request associated with the first possible dataset, the first request optionally selecting one or more of the one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3107 and/or 3108, receiving a first input associated with a first possible dataset comprises receiving a first request from a graphical user interface, and optionally from at least one submission element of a graphical user interface.

At the optional operation 3109 and/or 3110, receiving a first input associated with a first possible dataset comprises receiving a first request, the first request at least partially identifying one or more elements of the first possible dataset and/or optionally selecting one or more elements of the first possible dataset and/or optionally providing instructions at least partially identifying one or more elements of the first possible dataset.

At the optional operation 3111 and/or 3112, receiving a first input associated with a first possible dataset comprises receiving a first request, the first request providing instructions at least partially identifying one or more of the one or more target genetic characteristics and/or providing instructions for determining one or more of the one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 3113 and 3114, receiving a first input associated with a first possible dataset comprises receiving a first request associated with the first possible dataset 3113, and generating the first possible dataset in response to the first request, the first request optionally specifying one or more of the one or more target genetic characteristics and optionally at least one other instruction 3114. In some embodiments, receiving a first input associated with a first possible dataset comprises receiving a first request associated with the first possible dataset, the first request selecting and/or determining data representative of one or more of the one or more target genetic characteristics, and generating the first possible dataset in response to the first input.

In some embodiments, receiving a first input associated with a first possible dataset comprises receiving a first request from a graphical user interface, optionally from at least one submission element of a graphical user interface, optionally at least partially identifying one or more elements of the first possible dataset, and optionally selecting one or more elements of the first possible dataset, and generating the first possible dataset in response to the first input. In some embodiments, receiving a first input associated with a first possible dataset comprises receiving a first request from at least one submission element of a graphical user interface, the first request providing instructions identifying and/or determining data representative of one or more of the one or more target genetic characteristics, and generating the first possible dataset in response to the first input.

At the optional operations 3115 and 3116, receiving a first input associated with a first possible dataset comprises receiving a first request, the first request specifying data representative of one or more of the one or more target genetic characteristics 3115; and generating the first possible dataset in response to the first request at least partially by performing an analysis of data representative of the one or more target genetic characteristics 3116. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

In some embodiments, receiving a first input associated with a first possible dataset comprises receiving a first request, the first request specifying data representative of one or more of the one or more target genetic characteristics, and generating the first possible dataset in response to the first request at least partially by performing an analysis of data representative of one or more of one or more target nucleic acid sequences and/or target haplotypes.

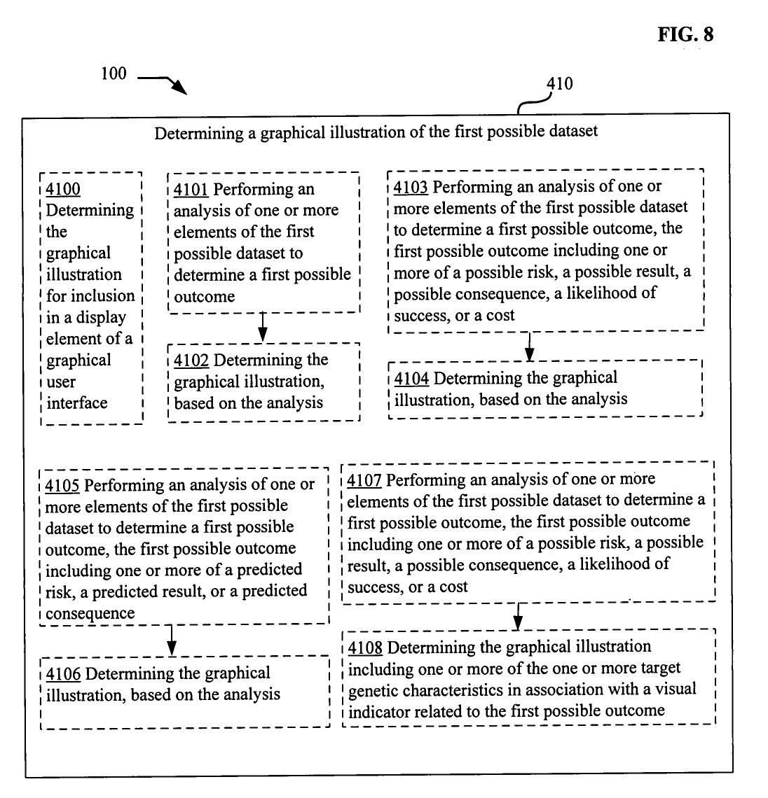

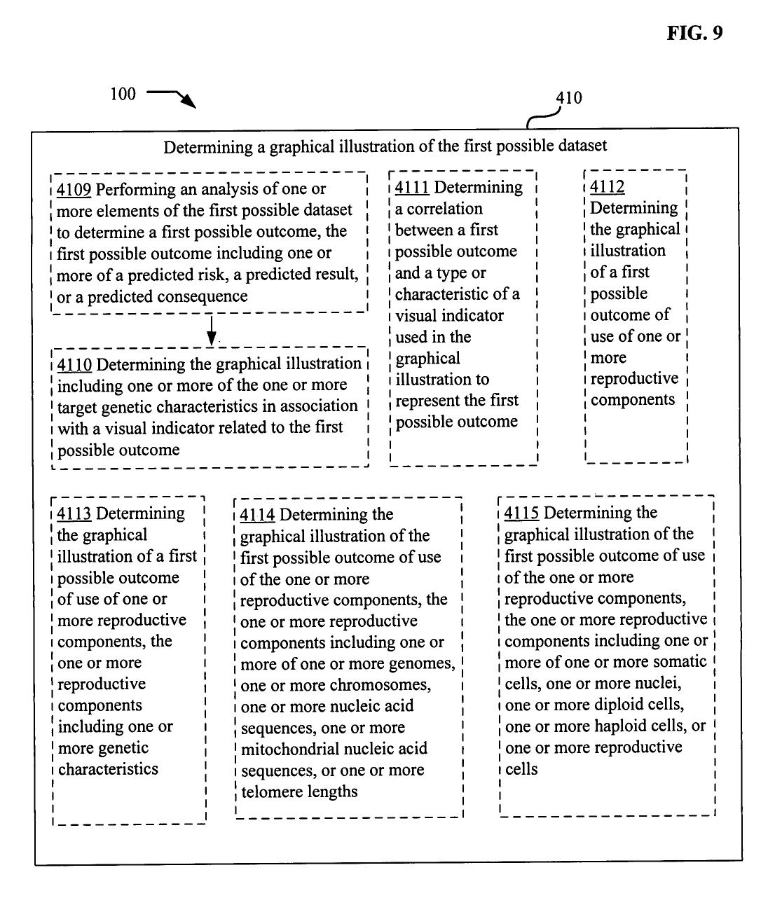

FIG. 8 and FIG. 9 illustrate optional embodiments of the operational flow 100 of FIG. 1. FIG. 8 and FIG. 9 show illustrative embodiments of the optional determining operation 410, including operations determining a graphical illustration of the first possible dataset, and may include at least one additional operation. Determining operations may optionally include, but are not limited to, operation 4100, operation 4101, operation 4102, operation 4103, operation 4104, operation 4105, operation 4106, operation 4107, operation 4108, operation 4109, operation 4110, operation 4111, operation 4112, operation 4113, operation 4114, and/or operation 4115.

At the optional operation 4100, determining a graphical illustration of the first possible dataset comprises determining the graphical illustration of the first possible dataset for inclusion in a display element of a graphical user interface.

At the operations 4101 and 4102, determining a graphical illustration of the first possible dataset comprises performing an analysis of one or more elements of the first possible dataset to determine a first possible outcome 4101; and determining the graphical illustration based on the analysis 4102.

At the optional operations 4103 and 4104, determining a graphical illustration of the first possible dataset comprises performing an analysis of one or more elements of the first possible dataset to determine a first possible outcome, the first possible outcome including one or more of a possible risk, a possible result, a possible consequence, a likelihood of success, or a cost 4103; and determining the graphical illustration based on the analysis 4104.

At the optional operations 4105 and 4106, determining a graphical illustration of the first possible dataset comprises performing an analysis of one or more elements of the first possible dataset to determine a first possible outcome, the first possible outcome including one or more of a predicted risk, a predicted result, a predicted consequence, a predicted likelihood of success, or a predicted cost 4105; and determining the graphical illustration based on the analysis 4106.

At the optional operations 4107 and 4108, determining a graphical illustration of the first possible dataset comprises performing an analysis of one or more elements of the first possible dataset to determine a first possible outcome, the first possible outcome including one or more of a possible risk, a possible result, a possible consequence, a likelihood of success, or a cost 4107; and determining the graphical illustration including data representative of one or more of the one or more target genetic characteristics in association with a visual indicator related to the first possible outcome 4108. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operations 4109 and 4110, determining a graphical illustration of the first possible dataset comprises performing an analysis of one or more elements of the first possible dataset to determine a first possible outcome, the first possible outcome including one or more of a predicted risk, a predicted result, a predicted consequence, a predicted likelihood of success, or a predicted cost 4109; and determining the graphical illustration including data representative of one or more of the one or more target genetic characteristics in association with a visual indicator related to the first possible outcome 4110. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 4111, determining a graphical illustration of the first possible dataset comprises determining a correlation between a first possible outcome and a type or characteristic of a visual indicator used in the graphical illustration to represent the first possible outcome.

At the optional operations 4112, 4113, 4114, and/or 4115, determining a graphical illustration of the first possible dataset comprises determining the graphical illustration of a first possible outcome based on use of one or more of the one or more reproductive components 4112, the one or more reproductive components optionally including one or more genetic characteristics 4113, optionally including one or more of one or more genomes, one or more chromosomes, one or more nucleic acid sequences, one or more mitochondrial nucleic acid sequences, and/or one or more telomeres and/or telomere lengths 4114, and/or optionally including one or more of one or more somatic cells, one or more germ line cells, one or more nuclei, one or more diploid cells, one or more haploid cells, and/or one or more reproductive cells 4115.

In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

In some embodiments, one or more of the one or more reproductive components include, but are not limited to, one or more of one or more spermatozoa, one or more spermatids, one or more spermatogonia, one or more primary spermatocytes, and/or one or more secondary spermatocytes. In some embodiments, one or more of the one or more reproductive components include, but are not limited to, one or more of one or more ova, one or more first polar bodies, and/or one or more second polar bodies.

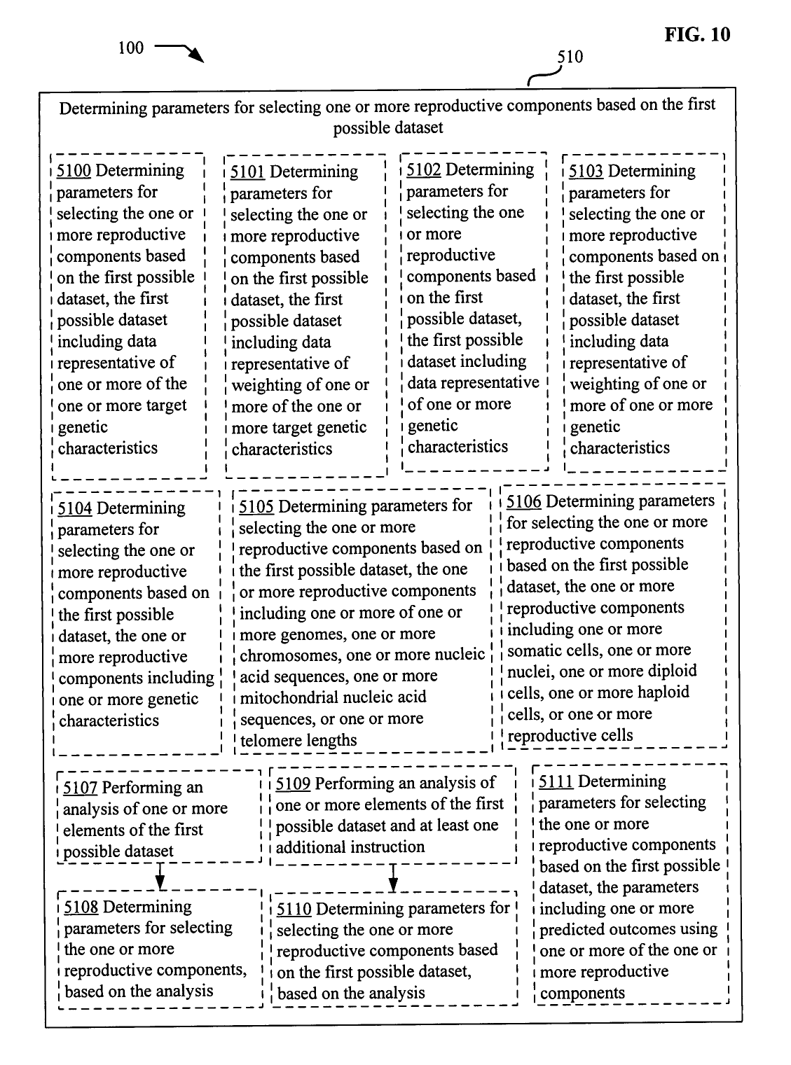

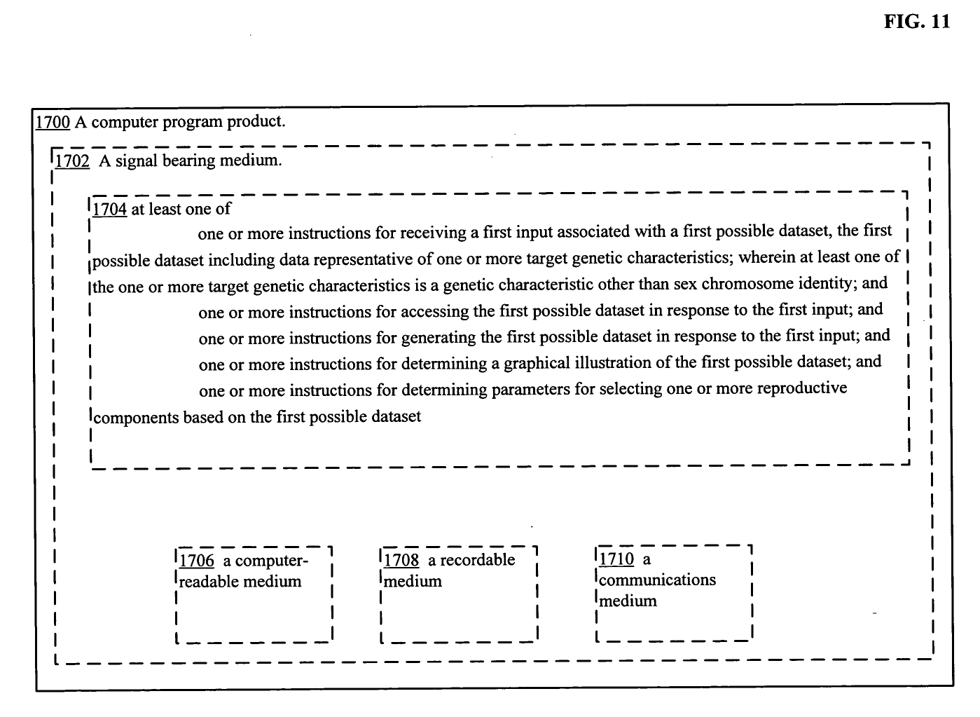

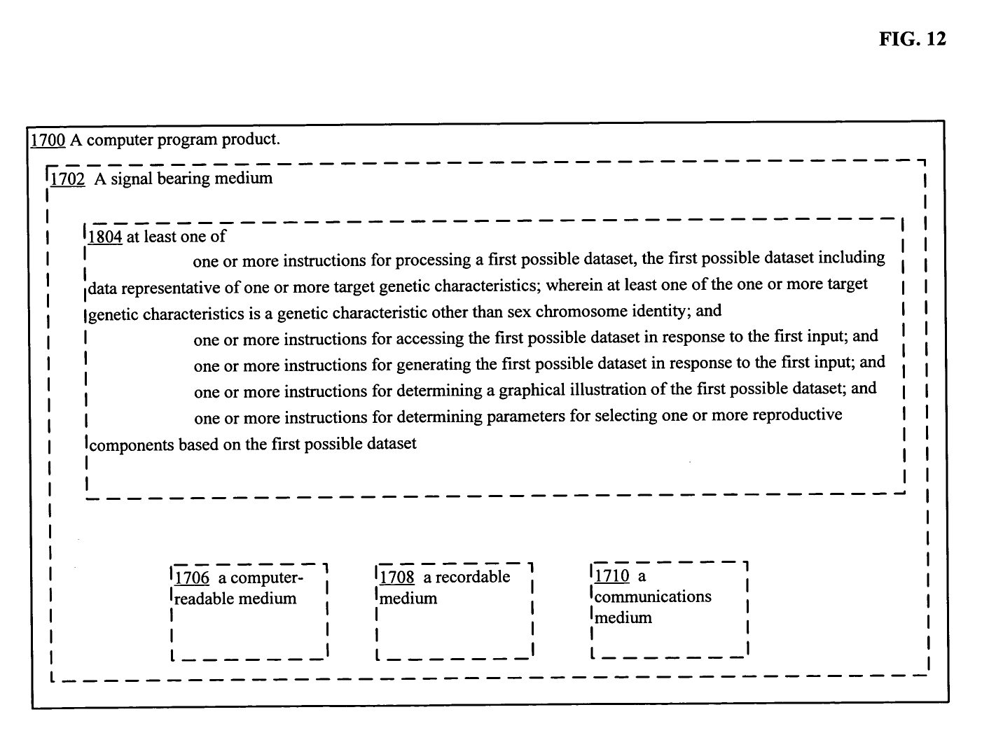

FIG. 10 illustrates optional embodiments of the operational flow 100 of FIG. 11. FIG. 18 shows illustrative embodiments of the determining operation 510, including operations determining parameters for selecting one or more reproductive components based on the first possible dataset, and may include at least one additional operation. Determining operations may optionally include, but are not limited to, operation 5100, operation 5101, operation 5102, operation 5103, operation 5104, operation 5105, operation 5106, operation 5107, operation 5108, operation 5109, operation 5110, and/or operation 5111.

At the optional operation 5100 and/or 5101, determining parameters for selecting one or more reproductive components based on the first possible dataset comprises determining parameters for selecting one or more reproductive components based on the first possible dataset, the first possible dataset including data representative of one or more of the one or more target genetic characteristics and/or weighting of one or more target genetic characteristics. In some embodiments, one or more of the one or more target genetic characteristics are selected from the group consisting of one or more genetic attributes, single nucleotide polymorphisms, haplotypes, allelic markers, alleles, disease markers, genetic abnormalities, genetic diseases, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 5102 and/or 5103, determining parameters for selecting one or more reproductive components based on the first possible dataset comprises determining parameters for selecting one or more reproductive components based on the first possible dataset, the first possible dataset including data representative of one or more of the one or more genetic characteristics and/or weighting of one or more of the one or more genetic characteristics. In some embodiments, one or more of the one or more genetic characteristics are selected from the group consisting of one or more single nucleotide polymorphisms, haplotypes, allelic markers, alleles, disease markers, genetic abnormalities, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements.

At the optional operation 5104 and/or 5105 and/or 5106, determining parameters for selecting one or more reproductive components based on the first possible dataset comprises determining parameters for selecting one or more reproductive components based on the first possible dataset, the one or more reproductive components including one or more genetic characteristics 5104, optionally including one or more of one or more genomes, one or more chromosomes, one or more nucleic acid sequences, one or more mitochondrial nucleic acid sequences, and/or one or more telomeres and/or telomere lengths 5105, and/or optionally including one or more of one or more somatic cells, one or more germ line cells, one or more nuclei, one or more diploid cells, one or more haploid cells, and/or one or more reproductive cells 5106.

In some embodiments, one or more of the one or more genetic characteristics are selected from the group consisting of single nucleotide polymorphisms, haplotypes, allelic markers, alleles, disease markers, genetic abnormalities, chromosomal abnormalities, genetic mutations, inversions, deletions, duplications, recombinations, chromosomes, nucleic acid sequences, genes, protein coding sequences, introns, exons, regulatory sequences, intergenic sequences, mitochondrial nucleic acid sequences, mitochondria, telomeres, telomere repeats, telomere lengths, centromere repeats, centromeres, methylation pattern, and epigenetic elements. In some embodiments, one or more reproductive components include, but are not limited to, one or more of one or more sperm, one or more spermatids, one or more spermatogonia, one or more primary spermatocytes, one or more secondary spermatocytes, one or more ova, one or more first polar bodies, and/or one or more second polar bodies.

At the optional operations 5107 and 5108, determining parameters for selecting one or more reproductive components based on the first possible dataset comprises performing an analysis of one or more elements of the first possible dataset 5107; and determining parameters for selecting one or more reproductive components, based on the analysis 5108.

At the optional operations 5109 and 5110, determining parameters for selecting one or more reproductive components based on the first possible dataset comprises performing an analysis of one or more elements of the first possible dataset and at least one additional instruction 5109; and determining parameters for selecting the one or more reproductive components, based on the analysis 5110.

At the optional operation 5111, determining parameters for selecting one or more reproductive components based on the first possible dataset comprises determining parameters for selecting one or more reproductive components based on the first possible dataset, the parameters including one or more predicted outcomes using one or more of the one or more reproductive components.

In some embodiments, determining parameters for selecting one or more reproductive components based on the first possible dataset comprises determining parameters for selecting the one or more reproductive components based on the first possible dataset, the parameters including one or more predicted outcomes selected from the group consisting of data characteristic of one or more of predicted risk, predicted result, predicted consequence, predicted likelihood of success, and predicted cost and/or data characteristic of weighting of one or more of predicted risk, predicted result, predicted consequence, predicted likelihood of success, and predicted cost. In some embodiments, determining parameters for selecting the one or more reproductive components based on the first possible dataset comprises determining parameters for selecting the one or more reproductive components based on the first possible dataset, the parameters including one or more predicted outcomes selected from the group consisting of data characteristic of one or more of a possible risk, a possible result, or a possible consequence and/or data characteristic of weighting of one or more of a possible risk, a possible result, or a possible consequence.

FIG. 11, FIG. 12, and/or FIG. 13 show a schematic of a partial view of an illustrative computer program product 1700 that includes a computer program for executing a computer process on a computing device. An illustrative embodiment of the example computer program product is provided using a signal bearing medium 1702, and may include at least one instruction of 1704, 1804, and/or 1904: one or more instructions for receiving a first input associated with a first possible dataset 1704, one or more instructions for processing a first possible dataset 1804, and/or one or more instructions responsive to a first possible dataset 1904, the first possible dataset including data representative of one or more target genetic characteristics, wherein at least one of the one or more target genetic characteristics is optionally a non-gender-specific genetic characteristic, a genetic characteristic other than sex chromosome identity, and/or a genetic characteristic other than gender; one or more instructions for accessing the first possible dataset in response to the first input; one or more instructions for generating the first possible dataset in response to the first input; one or more instructions for determining a graphical illustration of the first possible dataset; or one or more instructions for determining parameters for selecting one or more reproductive components based on the first possible dataset. The one or more instructions may be, for example, computer executable and/or logic implemented instructions. In some embodiments, the signal bearing medium 1702 of the one or more computer program 1700 products include a computer readable medium 1706, a recordable medium 1708, and/or a communications medium 1710.

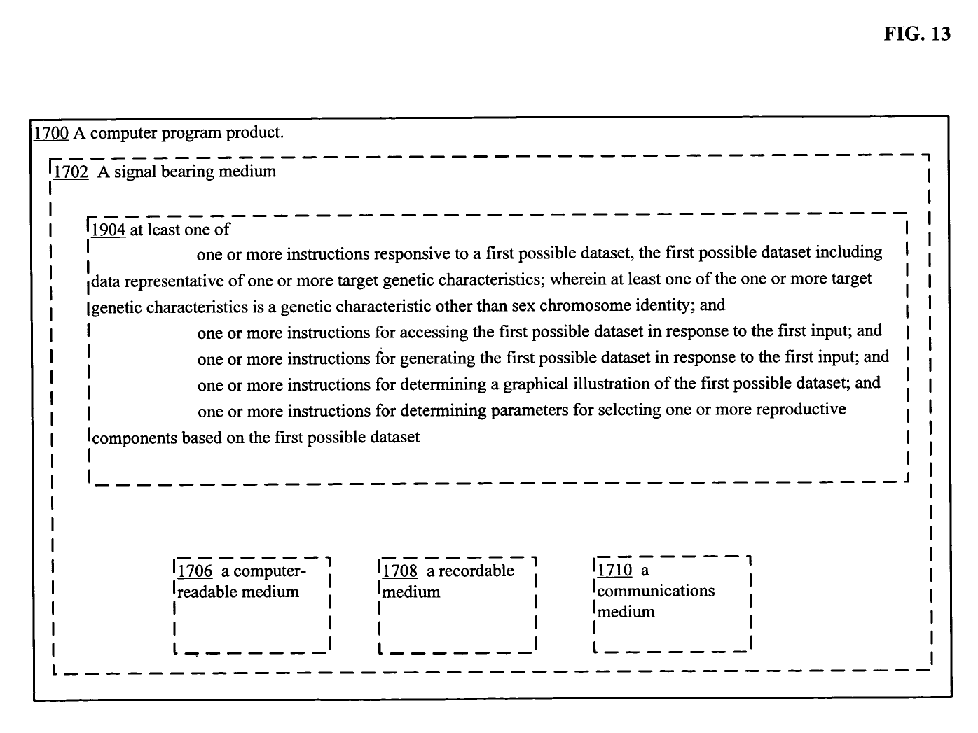

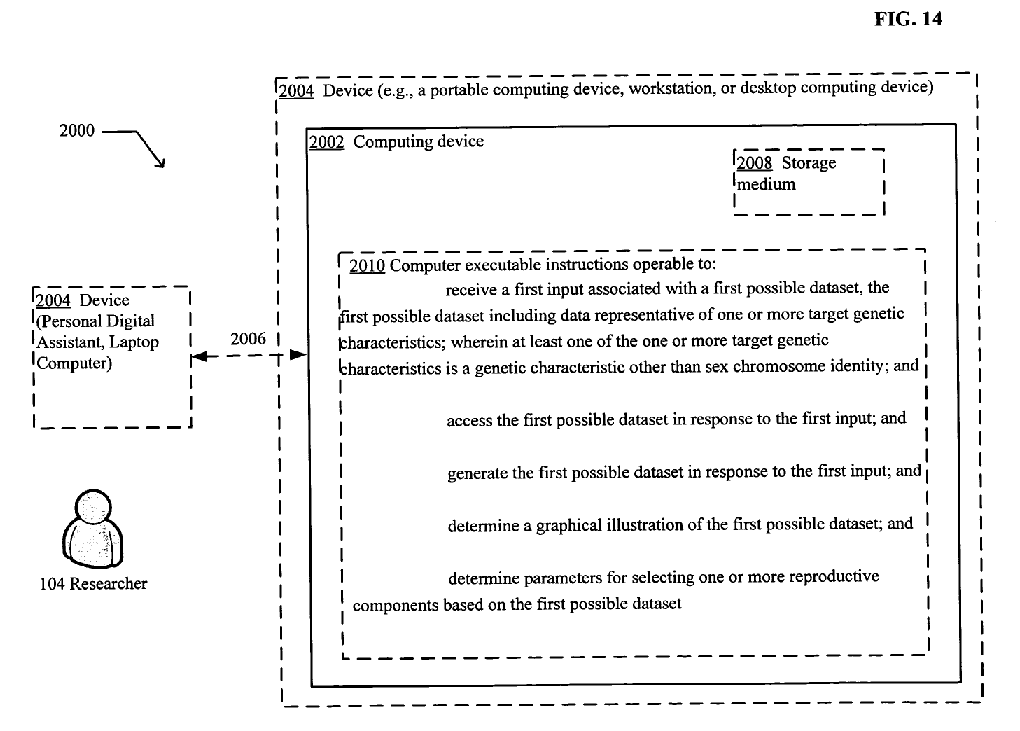

FIG. 14 shows a schematic of an illustrative system 2000 in which embodiments may be implemented. The system 2000 may include a computing system environment. The system 2000 also illustrates an operator and/or researcher 104 using a device 2004 that is optionally shown as being in communication with a computing device 2002 by way of an optional coupling 2006. The optional coupling may represent a local, wide area, or peer-to-peer network, or may represent a bus that is internal to a computing device (e.g. in illustrative embodiments the computing device 2002 is contained in whole or in part within the device 2004, one or more apparatus 410, one or more characterization units 419, one or more computing units 428, one or more controller units 426, one or more monitoring units 424, one or more hybridization units 422, one or more sequencing units 430, one or more amplifying units 432, and/or one or more decondensing units 434). An optional storage medium 2008 may be any computer storage medium.

The computing device 2002 includes one or more computer executable instructions 2010 that when executed on the computing device 2002 cause the computing device 2002 to receive the first input associated with the first possible dataset, the first possible dataset including data representative of one or more target genetic characteristics, optionally wherein at least one of the one or more target genetic characteristics is a genetic characteristic other than sex chromosome identity; optionally access the first possible dataset in response to the first input; optionally generate the first possible dataset in response the first input; optionally determine a graphical illustration of the first possible dataset; and determine parameters for selecting one or more reproductive components at least partially based on a first possible dataset. In some embodiments, at least one of the target genetic characteristics is a non-gender specific target characteristic, a genetic characteristic other than sex chromosome identity, and/or a genetic characteristic other than gender. In some illustrative embodiments, the computing device 2002 may optionally be contained in whole or in part within one or more units of an apparatus 410 of FIG. 15 (e.g. one or more characterization units 419, one or more computing units 428, one or more controller units 426, one or more monitoring units 424, one or more hybridization units 422, one or more sequencing units 430, one or more amplifying units 432, and/or one or more decondensing units 434), or may optionally be contained in whole or in part within the operator device 2004.

The system 2000 includes at least one computing device (e.g. 2004 and/or 2002 and/or one or more computing units 428 of FIG. 15) on which the computer-executable instructions 2010 may be executed. For example, one or more of the computing devices (e.g. 2002, 2004, 428) may execute the one or more computer executable instructions 2010 and output a result and/or receive information from the operator 104 (optionally from one or more apparatus 410, one or more characterization units 419, one or more controller units 426, one or more monitoring units 424, one or more hybridization units 422, one or more decondensing units 434, one or more sequencing units 430, and/or one or more amplifying units 432) on the same or a different computing device (e.g. 2002, 2004, 428) and/or output a result and/or receive information from an apparatus 410, one or more characterization units 419, one or more controller units 426, one or more monitoring units 424, one or more hybridization units 422, one or more decondensing units 434, one or more sequencing units 430, and/or one or more amplifying units 432 in order to perform and/or implement one or more of the techniques, processes, or methods described herein, or other techniques.

The computing device (e.g. 2002 and/or 2004 and/or 428) may include one or more of a desktop computer, a workstation computer, a computing system comprised a cluster of processors, a networked computer, a tablet personal computer, a laptop computer, or a personal digital assistant, or any other suitable computing unit. In some embodiments, any one of the one or more computing devices (e.g. 2002 and/or 2004 and/or 428) may be operable to communicate with a database to access the first possible dataset and/or subsequent datasets. In some embodiments, the computing device (e.g. 2002 and/or 2004 and/or 428) is operable to communicate with the apparatus 410.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those of skill in the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., ” a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those of skill in the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” It will be understood by one of skill in the art that “and/or” may mean the conjunctive “and” and in certain circumstances the disjunctive “or.” For example, “A and/or B” means any of “A and B” and “A or B.”

All references cited herein, including but not limited to patents, patent applications, and non-patent literature, are hereby incorporated by reference herein in their entirety.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
providing one or more female nuclei to one or more enucleated male germ line cells to obtain one or more female-nucleated male germ line cells;
inducing spermatogenesis in the one or more female-nucleated male germ line cells; and
selecting one or more of the one or more female-nucleated male germ line cells at least partially based on one or more genetic characteristics.

2. The method of claim 1, further comprising:
proliferating the one or more female-nucleated male germ line cells;

3. The method of claim 2, wherein the one or more female-nucleated male germ line cells are proliferated in vitro.

4. The method of claim 2, wherein the one or more female-nucleated male germ line cells are proliferated in situ.

5. The method of claim 1, wherein spermatogenesis is induced in vitro.

6. The method of claim 1, wherein spermatogenesis is induced in situ.

7. The method of claim 1, wherein the one or more female nuclei are from one or more germ line cells.

8. The method of claim 7, wherein the one or more germ line cells are one or more haploid germ cells.

9. The method of claim 7, wherein the one or more germ line cells are one or more diploid germ cells.

10. The method of claim 7, wherein the one or more germ line cells are one or more oocytes.

11. The method of claim 7, wherein the one or more germ line cells are one or more polar bodies.

12. The method of claim 7, wherein the one or more germ line cells are one or more stem cells.

13. The method of claim 1, wherein the one or more female nuclei are from one or more somatic cells.

14. The method of claim 13, wherein the one or more somatic cells are one or more stem cells.

15. The method of claim 13, wherein the one or more somatic cells are one or more progenitor cells.

16. The method of claim 1, wherein the one or more male germ line cells are stem cells.
The method of claim 1, wherein the one or more genetic characteristics are selected at least partially based on one or more genetic characteristics of one or more reference chromosomes.
The method of claim 1, wherein the one or more genetic characteristics are selected at least partially based on one or more genetic characteristics of one or more target chromosomes.

17. The method of claim 1, wherein selecting one or more of the one or more female-nucleated male germ line cells at least partially based on one or more genetic characteristics comprises:
selecting one or more of the one or more female-nucleated male germ line cells at least partially based on one or more genetic characteristics of one or more female germ line cells.

18. The method of claim 17, wherein the one or more genetic characteristics of the one or more female germ line cells include one or more genetic characteristics of one or more mitochondrial chromosome variants of the one or more female germ line cells.

19. The method of claim 17, wherein the one or more female germ line cells are one or more oocytes.

20. The method of claim 17, wherein the one or more female germ line cells are one or more polar bodies.

21. The method of claim 1, wherein selecting one or more of the one or more female-nucleated male germ line cells at least partially based on one or more genetic characteristics comprises:
selecting one or more of the one or more female-nucleated male germ line cells at least partially based on one or more genetic characteristics of one or more male germ line cells.

22. The method of claim 21, wherein the one or more male germ line cells are one or more spermatozoa.

23. The method of claim 21, wherein the one or more male germ line cells are one or more related spermatids.

24. The method of claim 21, wherein the one or more male germ line cells are one or more stem cells, wherein the one or more stem cells and the one or more male germ line cells are from a single donor.

25. The method of claim 1, further comprising:
providing the one or more female-nucleated male germ line cells to one or more oocytes for fertilization.

26. The method of claim 1, further comprising:
providing the one or more female-nucleated male germ line cells to one or more enucleated oocyte.

27. A method for optimizing female haploid genomes comprising:
providing one or more female nuclei to one or more enucleated male germ line cells to obtain one or more female nucleated male germ line cells;
inducing spermatogenesis in the one or more female-nucleated male germ line cells; and
selecting one or more of the one or more female-nucleated male germ line cells at least partially based on one or more genetic characteristics.

28. A composition of matter comprising:
one or more optimized female-nucleated male germ line cells, the optimized female-nucleated male germ line cells selected at least partially based on one or more genetic characteristics.
